(19) United States
(12) Patent Application Publication
(10) Pub. No.: US 2025/0195094 A1
(43) Pub. Date: Jun. 19, 2025

(54) Intravascular Lithoplasty System With Improved Durability, Efficiency and Pressure Output Variability

(71) Applicant: Cardiovascular Systems, Inc., Santa Clara, CA (US)

(72) Inventors: Tim Hidani, Mound, MN (US); Alex Thome, Minnetonka, MN (US); Daniel Schmitt, Belle Plaine, MN (US); Kevin Cornelius, Wayzata, MN (US); Sam Gardner, Crystal, MN (US); Michelle Peterson, Champlin, MN (US); Khama Chap, Corcoran, MN (US); Eric Kasen, Bismarck, ND (US)

(21) Appl. No.: 18/991,527

(22) Filed: Dec. 21, 2024

Related U.S. Application Data
(63) Continuation-in-part of application No. PCT/US2023/085868, filed on Dec. 23, 2023.
(60) Provisional application No. 63/654,375, filed on May 31, 2024.
(60) Provisional application No. 63/655,439, filed on Jun. 3, 2024.
(60) Provisional application No. 63/477,007, filed on Dec. 23, 2022.

(51) Int. Cl. A61B 17/22; A61B 17/00

(52) U.S. Cl. CPC A61B 17/22022; A61B 17/00234; A61B 2017/00243; A61B 2017/00305; A61B 2017/22025; A61B 2017/22038; A61B 2017/22062

(57) ABSTRACT

A catheter and segmented balloon system for an intravascular lithotripsy system having improved accommodation for curved configurations to prevent damage to the balloon during electrical arcing of electrode pairs. The catheter can include an elongate member with alternating flexibility regions wherein stiffer regions are configured to support at least one spaced-apart electrode pair, and wherein more flexible regions are disposed on either side of each stiffer region. This configuration allows the elongate member to curve more easily in the region of the electrodes, which are located within a balloon, or segmented balloon system. The balloon can include balloon segments with interposed sections between each of the segmented balloons wherein the outer diameter of an inflated balloon segment is larger than the outer diameter of the interposed section.

### CROSS-REFERENCE TO RELATED APPLICATIONS

This application claims priority to and benefit of (i) U.S. provisional application No. 63/654,375, filed May 31, 2024, and entitled INTRAVASCULAR LITHOTRIPSY CATHETER WITH IMPROVED DELIVERABILITY CHARACTERISTICS, (ii) U.S. provisional application No. 63/655,439, filed Jun. 3, 2024, and entitled INTRAVASCULAR LITHOTRIPSY CATHETER WITH IMPROVED DELIVERABILITY CHARACTERISTICS, (iii) and is a continuation-in-part of International application number PCT/US2023/085868, filed Dec. 23, 2023, and entitled Intravascular Lithoplasty System With Improved Durability, Efficiency and Pressure Output Variability, which claims priority to provisional application No. 63/477,007, filed Dec. 23, 2022, and entitled INTRAVASCULAR LITHOPLASTY AND/OR ANGIOPLASTY BALLOON SYSTEM WITH IMPROVED BALLOON MOUNT AND ELECTRODES, the entire contents of which applications are incorporated herein by reference as fully as if reproduced herein in full, for all purposes.

### STATEMENT REGARDING FEDERALLY SPONSORED RESEARCH OR DEVELOPMENT

None

### BACKGROUND

### Field

This disclosure relates to systems, devices and methods for breaking up calcified lesions in an anatomical conduit. In one aspect, an electrical arc is generated between two spaced-apart electrodes disposed within a fluid-filled member, creating pressure waves. In another aspect, a fluid-filled member may be inflated and deflated to open occluded blood vessels, including but not limited to calcified occlusions.

### Description of the Related Art

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than normal, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed pressure. The balloon forces expansion of the occlusion within the vessel and the surrounding muscular wall until the occlusion yields from the radial force applied by the expanding balloon, opening up the blood vessel with an inner diameter that is similar to the native vessel in the occlusion area and, thereby, improving blood flow. Generally, known IVL devices include a voltage pulse generator in operative communication with one or more pairs of electrodes mounted on a catheter and within an inflatable balloon.

Intravascular lithotripsy systems, devices and methods have been described in PCT/2022/074607, filed Aug. 5, 2022, and entitled “INTRA VASCULAR LITHOTRIPSY BALLOON SYSTEMS, DEVICES AND METHODS”, the entire contents of which are hereby incorporated by reference.

As illustrated in FIG. 1, a diagrammatic layout of portions of an exemplary IVL system 12 is provided. The illustrative IVL system 12 comprises a catheter assembly 114 including an elongate body, embodied as a catheter having guidewire 15, and a fluid-filled member 16 configured to contain conductive fluid therein, exemplified by an inflatable balloon, disposed near one end of the body and arranged to receive fluid for inflation to facilitate IVL therapy. A set of dischargeable spaced-apart electrodes 18 are shown arranged within the exemplary balloon 16, at least some of which are spaced apart by a gap 17 from each other to allow formation of a spark or electrical arc between the spaced-apart electrodes 18 when a voltage potential is provided between the electrodes 18.

The IVL system embodiments described herein may be used in connection with electrodes that are within a fluid-filled member 16 configured to contain a fluid, e.g., a conductive fluid, therein. The fluid-filled member 16 embodiments may include an inflatable balloon as shown in FIG. 1, which may be compliant or non-compliant and serves to contain the fluid such that the spaced-apart electrodes 18 are submerged within the contained fluid. In addition, the fluid-filled member 16 may comprise a fillable member that is at least partially rigid and/or not flexible. In other embodiments, the fluid-filled member 16 may contain the fluid therein and wherein the spaced-apart electrodes 18 are located or submerged within the contained fluid.

Alternatively, the IVL system control embodiments of the present disclosure may be used in connection with electrodes that are not located or surrounded by a fluid-filled or fillable member 16. In these embodiments, the IVL system may comprise spaced-apart electrodes 18 that may be continuously or periodically exposed to saline or other fluid and, during the exposure, the IVL system may generate an electrical arc between the spaced-apart electrodes 18.

each of the spaced-apart electrodes in FIG. 1 is arranged in communication (as suggested by dashed line conductors) with an electric pulse generation system, or voltage pulse generator, 20 to receive high voltage electrical energy for spark generation to create pressure waves for IVL therapy. In the illustrative embodiment, one electrode may be grounded and the other provided with high voltage from the electric pulse generation system 20, although in some embodiments, any voltage differential may be applied. The electric pulse generation system 20 includes an IVL control system 22 comprising a processor 24 configured for executing instructions stored on memory 26 and communications signals via circuitry 28 for IVL operations according to the processor governance. The processor 24, memory 26, and circuitry 28 are arranged in communication with each other (as suggested via dashed lines) to facilitate disclosed operations.

Appropriate control of such high-energy systems can also require achieving sufficient energy at the discharge site. Given the high-energy environment and microscale time periods for electronic discharge, desirable energy control within such IVL devices and systems can be challenging. Moreover, adaptable control methodologies may offer advantages to IVL effectiveness. Adjustable energy delivery can increase efficient power application, which can reduce risk to the patient. For example, beginning with a predetermined starting voltage threshold and defining a predetermined upper voltage threshold to form an acceptable voltage window may be proved. The acceptable voltage window may be coupled with series of generated voltage pulses of magnitudes that are confirmed to be within the acceptable voltage window. If, e.g., the magnitudes of the series of generated voltage pulses are below the predetermined upper voltage threshold, then the target voltage may be increased by a predetermined amount and another series of generated voltage pulses is executed. The embodiments of the IVL systems, devices and methods within the present disclosure includes operation for adjusting the total electrical energy provided to the set of electrodes for a given pulse. Such control systems for intravascular lithotripsy systems, devices and methods have been described in PCT/2023/79209, filed Nov. 9, 2023, and entitled “CONTROL OF IVL SYSTEMS, DEVICES AND METHODS THEREOF”, the entire contents of which are hereby incorporated by reference.

Additionally or alternatively, a different type of energy based emitter could be used in place of the emitter 18 to emit a high pressure wave, such as a shockwave, within fluid filled member 16. Examples of such emitters are shown in European patent published application number 571306 (laser); U.S. Pat. No. 10,201,387 and published U.S. Patent Application Number 2024/0016544 (laser or light energy), and U.S. Pat. No. 6,287,272 (ultrasound), the entire contents of which are hereby incorporated by reference.

A portion of an exemplary known competitive IVL device is shown in cross-section in FIG. 2, viewed in cross-section along a line cutting through an IVL balloon which surrounds, inter alia, a catheter body.

Thus, the prior art inflation port is in fluid communication with the fill lumen/passageway. The fill lumen/passageway of the competitive IVL device of FIG. 2 is formed between the inner surface of the catheter body and an outer surface of a sleeve that encompasses wire conductors (terminating at electrodes that are located along the catheter and within the balloon). Thus, the wire conductors of the known device are not exposed to the fluid within the fill lumen/passageway. Electrodes that are located within the balloon and electrically connected with the wire conductors are not covered by the encompassing sleeve and, therefore, are exposed to fluid within the balloon. In addition, the known competitive device comprises an angioplasty balloon that is adhered at both the proximal end of the balloon and the distal end of the balloon to an outer surface of the catheter body, with the fill lumen/passageway into the balloon's interior being formed within the catheter body and defined by a space between the sleeve and the inner surface of the catheter body. Accordingly, the competitive known IVL balloon is adhered at its proximal end and its distal end to one structure, that is, the outer surface of the catheter body.

In addition, the competitive known IVL device of FIG. 2 comprises the catheter body, and the guidewire member (which defines the guidewire lumen) both extending through the balloon. Both of these structures continue distally beyond a distal end of the balloon to a distal tip. As a result, the distal tip of the known competitive device comprises a stiffness and deformability that can be improved upon to, in turn, improve the device's ability to translate tortuous vasculature and reduce the potential of damaging the vasculature during translation.

A portion of the competitive known IVL device is shown in cross-section in FIG. 2, viewed in cross-section along a line cutting through an IVL balloon which surrounds, inter alia, a catheter body. One manual measurement, using a destructive method to cut a section of the balloon to measure, using a calibrated micrometer has shown that one type of a known competitive IVL device has a single wall thickness of about 0.001″. Another measurement of a different known competitive IVL device yielded a single wall thickness result of about 0.0013″.

Further, the sleeve shown in FIG. 2 adds a layer of material that adds crossing profile thickness, increases complexity and takes up a portion of the area of the fill lumen/passageway, thereby reducing the available volume fill lumen/passageway during inflation and/or deflation cycles.

FIG. 3 graphically illustrates the drop in impedance of a current leader across a spark gap defined between two spaced-apart electrodes in an IVL system such as shown in FIG. 1 following application of voltage to one of two spaced-apart electrodes as the current leader develops into an electrical arc between the spaced-apart electrodes. This, in turn, causes the power dissipated in the electrical arc to peak sharply while the voltage and current between the electrodes are both relatively high. The current reaches a peak and the voltage drops, both very rapidly, indicating that an electrical arc between the spaced-apart electrodes is present, or has occurred. The peak of the power dissipated in the electrical arc indicates the relatively short time interval during which all the useful work of heating the growing leader into an arc is performed. The graphic illustration of FIG. 3 is exemplary of one aspect of an IVL procedure that produces pressure waves.

It would be advantageous to provide an IVL system and/or device that reduces the risk of trauma during translation of the angioplasty balloon to an anatomical location of interest and that reduces the complexity of construction of a lithotripsy system.

Moreover, it would be advantageous to provide an IVL system and/or device that comprises a reduced crossing profile, a more flexible tip and a kink-resistant shaft.

Known IVL devices also comprise a spark gap between electrode pairs that, when a sufficiently high voltage is applied to a first electrode, facilitates a spark or electrical arc of current from the first electrode across the spark gap to a second electrode in the electrode pair. This process results in loss of material, or erosion, from each of the electrodes in the electrode pair. Known spark gaps are generally arranged axially, i.e., with terminal faces of wire conductors spaced apart from each other and facing each other in an axial spark gap configuration. In this case, the electrical arc involves the terminal or distal face of at least one electrode in the electrode pair. Continued arcing across the spark gap during an IVL procedure with these known devices results in erosion of material from each electrode involved in the electrical arc. Each of these arcs causes the spark gap to slightly increase in size and/or short, with the full procedural set of arcs resulting in an appreciable increase in spark gap size which may lead to unpredictability in generating an electrical arc. Alternatively, 1st and 2nd electrodes may be arranged concentrically, with the spark gap defined in a radial direction, wherein the 1st and 2nd electrodes are not arranged along a common radial or circumferential plane. As will be demonstrated herein, this arrangement generates pressure outputs with relatively high variability. In addition, known IVL devices produce a resultant pressure output, or “shockwave” or “pressure wave” that over a series of electrical arcs, decreases in magnitude as the IVL procedure is executed. Moreover, the highly variable pressure outputs produced by the known IVL devices can lead to unpredictable, or undesirable, outcomes and perhaps contributes to balloon instability over time due to the variability of the pressure outputs placing stress on the balloon material. Further, known IVL coronary devices are configured to produce a total maximum number of 120 pulses per catheter at a frequency of 1 Hz. Known IVL peripheral device are configured to produce a total maximum number of 300 pulses per catheter at 1 Hz.

It would be advantageous to provide an IVL device or system that is designed to maintain a desired spark gap distance between spaced-apart electrodes across the execution of a full IVL procedure and produce tightly controlled output pressures having a much tighter data spread from high to low data points, and lower standard deviation as compared to the known IVL devices.

It would also be desirable to provide an IVL device or system that is more durable and more efficient than known devices through structural and operational improvements to provide an IVL device or system that can provide up to, and more than, 300 voltage pulses per catheter in some embodiments and in other embodiments up to 500 voltage pulses per catheter, with a frequency of 1 to 5 Hz, with 2 Hz being a preferred frequency.

It would be advantageous to provide an IVL device or system with a catheter comprising key features that allow for improved pushability and kink resistance, particularly in the region of the balloon, as well as a reduced crossing profile in at least that region. It also would be advantageous to provide a catheter for an IVL system that provides improved deliverability characteristics such as a smaller crossing profile than known IVL systems, as well as improved flexibility and pushability.

Various embodiments of the present disclosure address one or more of the advantages, among others, discussed above.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a schematic view of an exemplary IVL device.

FIG. 2 illustrates a cutaway view of a known IVL device.

FIG. 3 illustrates a graphic illustration of a typical timing of applied voltage and current during application of voltage and generation of an electrical arc between spaced-apart electrodes.

FIG. 4 illustrates one embodiment of the present disclosure.

FIG. 5 illustrates one embodiment of the present disclosure.

FIG. 6 illustrates a side, cutaway view of a distal portion of an exemplary embodiment of the present disclosure.

FIG. 7A illustrates a side, cutaway view of an exemplary embodiment of the present disclosure.

FIG. 7B illustrates a side, cutaway view of an exemplary embodiment of the present disclosure.

FIG. 8 illustrates a side, cutaway view of a portion of the distal region of an exemplary embodiment of the present disclosure.

FIG. 9 illustrates a partial cutaway view of a portion of an exemplary embodiment of the present disclosure.

FIG. 10 illustrates a side, cutaway view of a portion of FIG. 6.

FIG. 11 illustrates a side, cutaway view of the embodiment of FIG. 10.

FIG. 12A illustrates an embodiment of a portion of a system of the present disclosure.

FIG. 12B1 illustrates an embodiment of a portion of a system of the present disclosure.

FIG. 12B2 illustrates an expanded view of a portion of a system of the present disclosure.

FIG. 12B3 illustrates an expanded view of a portion of a system of the present disclosure.

FIG. 12C illustrates an embodiment of a portion of a system of the present disclosure.

FIG. 12D illustrates an embodiment of a portion of a system of the present disclosure.

FIG. 12E illustrates a cross-sectional view of a portion of a system of the present disclosure.

FIG. 12F illustrates a cross-sectional view of a portion of a system of one embodiment of the present disclosure.

FIG. 12G illustrates a cross-sectional view of a portion of a system of one embodiment of the present disclosure.

FIG. 13A illustrates an embodiment of a portion of a system of the present disclosure.

FIG. 13B illustrates an embodiment of a portion of a system of the present disclosure.

FIG. 14A illustrates a side, cutaway view of one embodiment of the present disclosure.

FIG. 14B illustrates a portion of device illustrated in FIG. 14A.

FIG. 15A illustrates one embodiment of the present disclosure.

FIG. 15B illustrates one embodiment of the present disclosure.

FIG. 15C illustrates one embodiment of the present disclosure.

FIG. 15D illustrates one embodiment of the present disclosure.

FIG. 15E illustrates a cross-sectional view of one embodiment of the present disclosure.

FIG. 16A illustrates one embodiment of the present disclosure.

FIG. 16B illustrates one embodiment of the present disclosure.

FIG. 16C illustrates one embodiment of the present disclosure.

FIG. 16D illustrates one embodiment of the present disclosure.

FIG. 17A illustrates one embodiment of the present disclosure.

FIG. 17B illustrates one embodiment of the present disclosure.

FIG. 17C illustrates one embodiment of the present disclosure.

FIG. 17D illustrates one embodiment of the present disclosure.

FIG. 18A illustrates one embodiment of the present disclosure.

FIG. 18B illustrates one embodiment of the present disclosure.

FIG. 19A illustrates one embodiment of the present disclosure.

FIG. 19B illustrates one embodiment of the present disclosure.

FIG. 20 illustrates a side view of one embodiment of the present disclosure.

FIG. 21 illustrates a schematic diagram of one embodiment of the present disclosure.

FIG. 22 illustrates a top cutaway view of one embodiment of the present disclosure.

FIG. 23 illustrates a block diagram of one embodiment of the present disclosure.

FIG. 24 illustrates a pressure plot comparing test and known IVL devices.

FIG. 25 illustrates a force tracking plot comparing tracking force through a tracking fixture for TEST and KNOWN IVL catheters.

FIG. 26A illustrates an IVL catheter of one embodiment of the present disclosure.

FIG. 26B illustrates schematically a testing system for testing an IVL catheter of one embodiment of the present disclosure.

FIG. 27 illustrates a graphical illustration of 3-point bend testing of one embodiment of the present disclosure,

FIG. 28 illustrates a graphical illustration of 3-point bend testing of one embodiment of the present disclosure.

FIG. 29 illustrates a graphical illustration of 3-point bend testing of one embodiment of the present disclosure.

FIG. 30 illustrates a graphical illustration of 3-point bend testing of one embodiment of the present disclosure.

FIG. 31 illustrates a graphical illustration of 3-point bend testing of one embodiment of the present disclosure.

FIG. 32 illustrates an IVL catheter of one embodiment of the present disclosure.

FIG. 33 illustrates a cross-sectional view of a first point of the IVL catheter of FIG. 32 of one embodiment of the present disclosure.

FIG. 34 illustrates a cross-sectional view of a second point of the IVL catheter of FIG. 32 of one embodiment of the present disclosure.

FIG. 35 illustrates a cross-sectional view of a third point of the IVL catheter of FIG. 32 of one embodiment of the present disclosure.

FIG. 36A illustrates a portion of a rapid exchange port of the IVL catheter of FIG. 32 of one embodiment of the present disclosure.

FIG. 36B illustrates a cross-sectional view of the rapid exchange port of the IVL catheter of one embodiment of the present disclosure.

FIG. 36C illustrates a cross-sectional view of a fourth point of the IVL catheter of FIG. 32 of one embodiment of the present disclosure.

FIG. 37 illustrates a cross-sectional view of a fifth point of the IVL catheter of FIG. 32 of one embodiment of the present disclosure.

FIG. 38 illustrates a cross-sectional view of a sixth point of the IVL catheter of FIG. 32 of one embodiment of the present disclosure.

### DETAILED DESCRIPTION

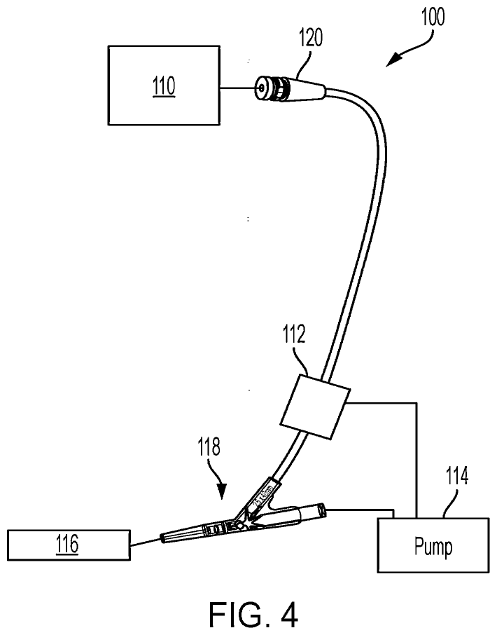

FIG. 4 illustrates an embodiment of an IVL system 100 of the present disclosure. A voltage pulse generator 110 is provided in operative connection and association and/or communication with a controller 112 configured in some embodiments to provide programmed operative instructions to the voltage pulse generator 110 and, in some embodiments, to a fluid reservoir/fluid pump or infusion device 114. In some embodiments, an EPROM may be provided for provision of programmed operative instructions, wherein the EPROM may be located on or with a handle of the system 100. As illustrated, the the controller 112 and voltage pulse generator 110 are in operative electrical communication and/or association with wire conductors and pairs of electrodes as discussed supra. The wire conductors may be disposed along the catheter's length, e.g., the shaft of FIG. 4 and wherein the electrode pairs are located within an interior of an inflatable enclosure, such as, for example, a balloon, positioned at or near a distal end of the catheter structure. The catheter and balloon structure are represented schematically by element number 116. A hub 118 allows operative connection and association and/or communication with the controller 112, the voltage pulse generator 110, and, in some embodiments, the fluid reservoir/pump or infusion device 114. In addition, the illustrated connector 120 is in operative connection and communication with the controller 112 and can provide operative electrical connection and communication with the wire conductors and electrode pairs. The controller 112 may comprise a processor for executing programmed instructions, for example initiating voltage pulses at a predetermined magnitude and frequency and in a predetermined pattern of pulses and magnitudes. The processor may be in operative communication and connection with a memory and a display. In some embodiments, the hub 118 may allow for over-the-wire guidewire access through a lumen defined within the catheter. In a preferred embodiment, a rapid exchange (RX) access is provided. Some embodiments of the controller 112 may comprise an EPROM comprising programmed instructions, for example and without limitation, initiating voltage pulses at a predetermined magnitude and frequency and in a predetermined pattern of pulses and magnitudes.

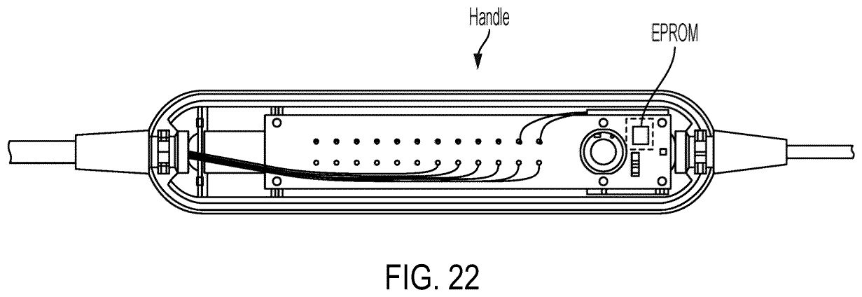

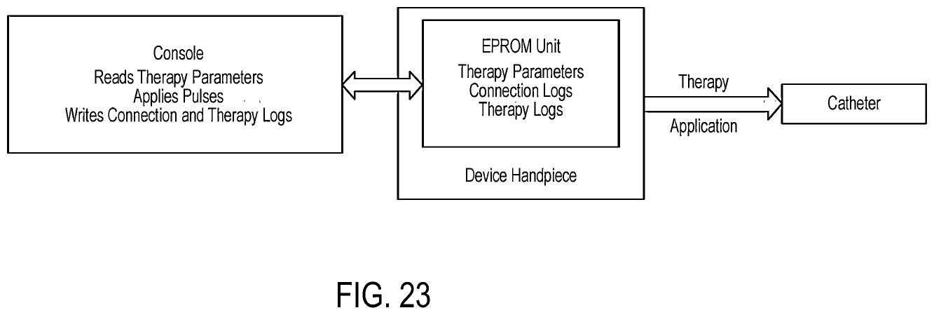

As discussed in FIGS. 22 and 23, some embodiments may comprise a handle that may comprise an EPROM comprising programmed instructions, for example and without limitation, initiating voltage pulses at a predetermined magnitude and frequency and in a predetermined pattern of pulses and magnitudes. The EPROM may be in operative communication and connection with a console comprising a processor for executing programmed instructions, a memory in operative communication with the processor and, in some embodiments, the EPROM, and a display. In some embodiments, the hub 118 may allow for over-the-wire guidewire access through a lumen defined within the catheter.

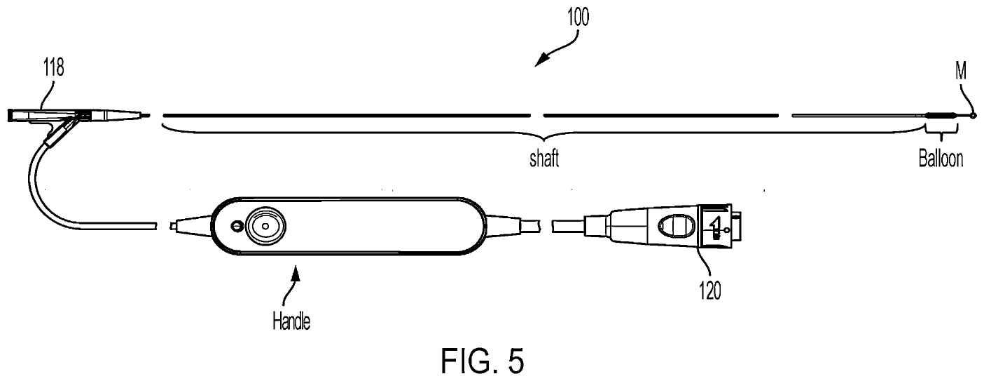

FIG. 5 shows a broken side view of an IVL system 100 comprising a handle with a connector 120 configured to connect with the voltage pulse generator 110 of FIG. 4 and a console or controller such as 112 of FIG. 4. A removable mandrel M is shown inserted through a guidewire lumen defined along at least a portion of the catheter shaft. In FIG. 4, the removable mandrel M is shown extending proximally through a flexible distal tip and through the defined guidewire lumen of the catheter.

Additionally, or alternatively, instead of the removable mandrel M, the catheter shaft may provide the stiffness based on the material thereof. According to this embodiment, the catheter shaft may fill the inside thereof except some lumens. The number of lumens may be two for two wire conductors in a portion of the catheter shaft, three for the two wire conductors and a guidewire in another portion, and four for the two wire conductors, the guidewire, and saline solution in the other portion. It will also be understood that in other configurations the number of lumens can exceed four lumens or be greater than four lumens and that those four or more lumens can be disposed at other locations within the catheter than those illustrative examples above. For instance, and not by way of limitations, 2 or more lumens can be found in different portions of the catheter shaft with those 2 or more lumens accommodating wire conductors, guidewire(s), saline solution, etc. Additionally, while different portions are described as having different numbers of lumens, it will be understood that the catheter can include the same number or a different number of lumens along all or portions of the catheter.

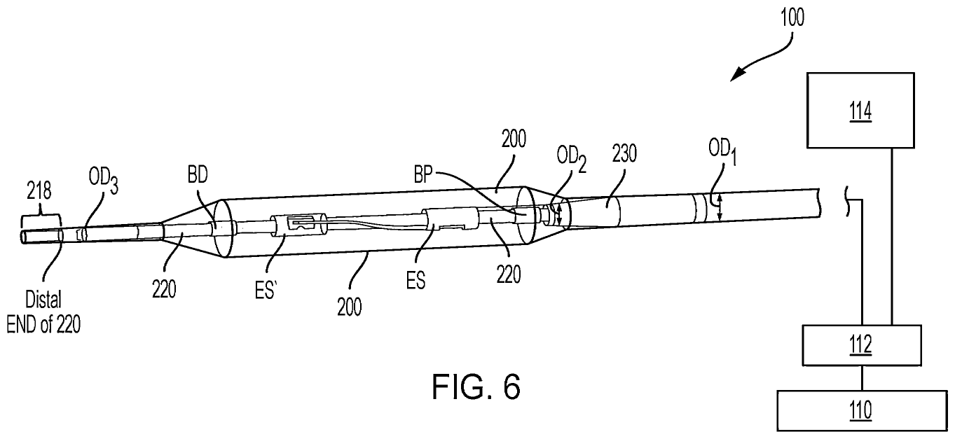

Distal portion embodiments of the catheter and balloon element 116 of the exemplary IVL system 100 of FIGS. 4 and 5 are illustrated in FIGS. 6-11.

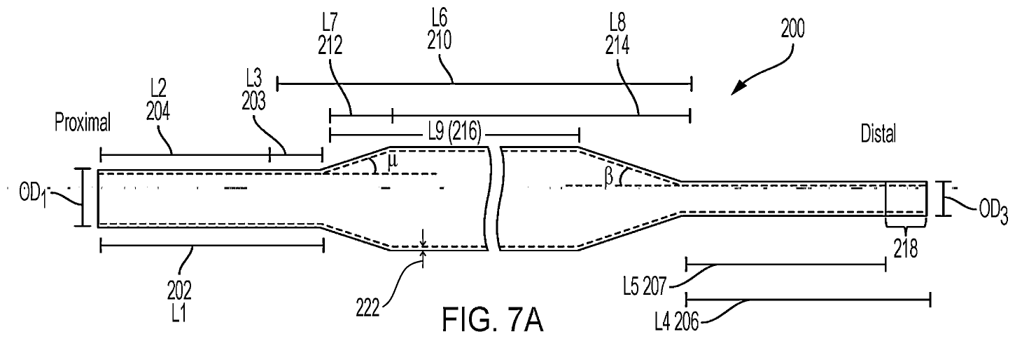

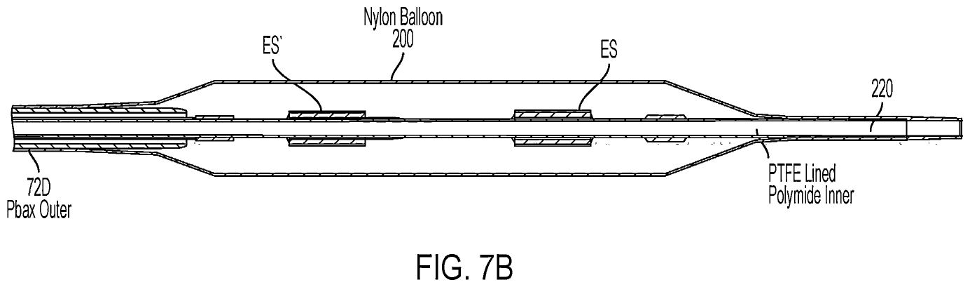

As best seen in FIG. 7A, an inflatable balloon 200 is provided and comprises a cylindrical proximal section 202, a cylindrical distal section 206 and an inflatable portion 210 comprising an unbonded section 203 of the cylindrical distal section 206, a tapering proximal section 212, a tapering distal section 214 and a substantially cylindrical section 216 disposed between the tapering proximal and distal sections 212, 214.

A proximal portion 207 of the cylindrical distal section 206 of the balloon 200 surrounds, and is bonded or sealed in a watertight engagement against an outer surface of an elongate member 220 as best shown in FIG. 6. A distal region of the cylindrical distal section 206 extends, e.g., is extruded, beyond a distal end of the elongate member 220 to form an atraumatic, flexible tip 218.

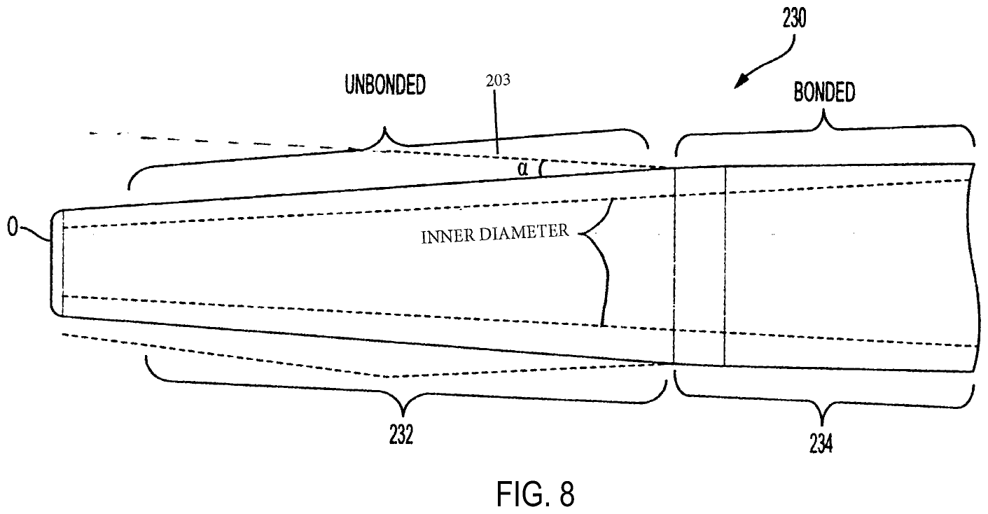

A proximal portion 204 of the cylindrical proximal section 202 of the balloon surrounds, and is sealed or bonded in a watertight engagement against, the non-tapering outer surface 234 of the tapering outer member 230 (see FIG. 8), and is not sealed or bonded against any portion of the elongate member 220 which is received within, and extends distally from, a distal end of the tapering outer member 230. A distal length comprising an unbonded section 203 of the cylindrical proximal section 202 of the balloon 200 surrounds the distal tapering section 232 of the tapering outer member 230, but is not bonded or sealed against any portion of the tapering outer member 230. As a result, the inflatable section of the balloon 200 comprises: the unbonded section 203 of the cylindrical proximal section 202, the proximal tapering section 212, the distal tapering section 214 and the substantially cylindrical section 216 disposed between the proximal and distal tapering sections 212, 214.

The tapering outer member 230 comprises an outer diameter that is larger than an outer diameter of the elongate member 220, and is configured to receive the elongate member 220.

In addition, a length of a fluid conveying pipe P is defined along the length of the device and between the concentric arrangement of the outer surface of the elongate member 220 and the inner surface of the tapering outer member 230. The remaining portion of the fluid conveying pipe P is defined proximally along the catheter shaft. The fluid conveying pipe P is a combination of the inside space or lumen of the elongate member 220 for the fluid, the inside of the balloon 200, which is fluidically communicated with the inside space or lumen of the elongate member 220, and any connecting paths therebetween or extending therefrom. The fluid conveying pipe P is provided the exclusive path for fluid communication between the fluid reservoir/pump 114 and the inflatable section 210 of the balloon 200. The fluid conveying pipe P terminates distally relative to the distal end of the tapering outer member 230 where an opening O is defined for fluid flow into, and out of, the balloon 200.

It is significant to note that the distal end of the tapering outer member 230, and therefore the opening O for fluid flow, extends distally beyond a distal end of the proximal cylindrical section 202 of the balloon. As a result, the proximal cylindrical section 202 of the balloon surrounds at least a portion of the proximal non-tapering, or cylindrical section, 234 of the tapering outer member 230, and is sealed or bonded against the non-tapering or cylindrical (proximal) section's 234 outer surface in a watertight seal. A distal end region of the tapering outer member 230 is, however, not surrounded by the proximal cylindrical section 202 of the balloon. Instead, the distal end region of the tapering outer member 230, and its opening O, extends into the proximal tapering section 212 of the balloon 200.

As a result of the watertight sealing mechanism described above, and the location of the opening O for fluid flow into and out of the inflatable portion 210 of the balloon 200, the proximal cylindrical section 202 of the balloon 200 plays no role in the movement of fluid into or out of the inflatable portion 210 of the balloon 200. Similarly, the inflatable portion 210 of the balloon 200 also plays no role in the inflation/deflation or degassing of the fluid, or the movement of fluid into or out of the inflatable portion 210 of the balloon 200. The inflatable portion 210 simply receives the incoming fluid from, or delivers outgoing fluid into, the opening O at the distal end of the tapering outer member 230.

FIG. 8 shows a distal portion of the tapering outer member 230, including a tapering section 232 comprising a tapering angle α. The tapering section 232 in this embodiment comprises the unbonded section 203 of the balloon 200, wherein the proximal cylindrical section 203 of balloon 200 (see FIG. 7A) surrounds a portion of the tapering section 232, but is not adhered or bonded or sealed against the outer surface of the tapering section 232. Proximal of the tapering section 232 the tapering outer member 230 comprises a cylindrical section 234 of substantially constant diameter. This is indicated as the bonded section and provides an outer surface which the proximal portion 204 of the proximal cylindrical section 202 of the balloon 200 surrounds and is sealed or bonded in a watertight engagement. Sides of the fluid conveying pipe P is discussed above comprise a resulting inner diameter that is shown in dashed lines, wherein the pipe P terminates at an opening O at the distal end of the tapering outer member 230. As shown by the inner set of dashed lines, the inner diameter tapers distally within the tapering section 232. In other embodiments, the pipe P may be of substantially constant diameter through both the cylindrical section 234 and the tapering section 232, wherein the wall thickness of the pipe may neck down, or be made thinner moving in the distal direction at tapering section 232 while retaining a constant inner diameter through the cylindrical section 234 and the tapering section 232. In other embodiments, as in FIG. 8, the contours of the pipe P may taper downward with the tapering angle α of the tapering section 232. In some embodiments the inner diameter of the pipe P may comprise a constant diameter.

As shown in FIGS. 7A and 7B, and with continued reference to FIGS. 6, 8 10 and 11, the cylindrical proximal section 202 of the balloon 200 comprises a length L1 and an outer diameter OD1. Even though the length L1 of the cylindrical proximal section 202 surrounds the tapering outer member 230, only a proximal portion 204 of the cylindrical proximal section 202 is bonded or sealed in a watertight configuration against an outer surface of the elongate member. The proximal portion 204, also referred to as the proximal watertight sealed or bonded portion 204 has a length L2 which is less than L1. Finally, the cylindrical proximal section 202 further comprises, on its distal end, an unbonded section 203 having a length of L3 and that surrounds the tapering outer member 230, but is not bonded or sealed to the elongate member, wherein length L3 is less than L2 and L1, such that L2 plus L3 equals L1.

The balloon 200 further comprises a cylindrical distal section 206 having an overall length of L4, including the distal tip 218. Without including the distal tip 218, the proximal portion 207 of the cylindrical distal section 206 that surrounds, and is sealed or bonded in a watertight engagement against the outer surface of, the elongate member 220 comprises a length of LS, which is shorter than L4. Accordingly, the distal tip 218 extends distally beyond the distal end of the elongate member 220 for a distance to provide an atraumatic tip and to facilitate translation through the vasculature.

As described above, the inflatable portion 210 of the balloon 200 comprises the unbonded section (length L3) 203, the tapering proximal section 212 and the tapering distal section 214, and the substantially cylindrical section 216 disposed therebetween. Accordingly, the length of the inflatable section is L6 which includes the unbonded section 203 of the proximal cylindrical section of length L3, the proximal tapering section 212 of length L7, the distal tapering section 214 of length L8, and the substantially cylindrical section 216 of length L9, for an overall inflatable section length of L6.

The tapering outer member 230 may taper down to a smallest outer diameter OD2 at its distal end that is smaller than outer diameter OD1 which is the effective outer diameter of the proximal cylindrical section 202 as well as the outer diameter of the non-tapering portion 234 of the tapering outer member 230. In this embodiment, an inner diameter of the balloon's cylindrical proximal section 202 may be substantially equivalent to the outer diameter of the tapering outer member at OD1, which is the non-tapering portion 234 of the tapering outer member to which the proximal cylindrical section is watertight bonded or scaled. Similarly, an inner diameter of the proximal portion 207 of the balloon's cylindrical distal section 206 that is watertight bonded or sealed to the elongate member 220 may be substantially equivalent to the outer diameter of the elongate member 220 at OD3.

As can be seen in FIGS. 6 and 7A, the outer diameter of the outer member to which the balloon's cylindrical proximal section is sealed or bonded, that is OD1, may be greater than the outer diameter of the elongate member with outer diameter OD3.

As noted, the elongate member 220 is received within tapering outer member 230. As a result, the balloon 200 is sealed against two distinct structures. On the proximal side, the balloon 200 is sealed against the non-tapering outer surface 234 of the outer member 230 while on the distal side, the balloon 200 is sealed against the outer surface of the elongate member 220.

The balloon's distal tip 218 is preferably flexible and comprises a conduit defined therethrough and that aligns with a conduit defined through the elongate member to allow for, inter alia, guidewire access.

In some embodiments, the larger proximal side outer diameter OD1 as compared with smaller distal side outer diameter OD3, may produce a tapering angle μ for the tapered proximal section of the balloon that is different from, e.g., smaller than, the tapering angle β for the tapered distal section of the balloon. These tapering angles are measured with reference to the dashed lines of FIG. 7A, which are collinear with the non-tapering outer surface of the outer member (tapering angle μ, and the outer surface of the elongate member (tapering angle β)). In some embodiments, the length L7 of the tapered proximal section may be shorter than the length LS of the tapered distal section.

Because of these exemplary relative dimensions, as shown in both FIGS. 7A and 7B, embodiments of the balloon 200 in the inflatable section 210 may be longitudinally asymmetric. In particular, the inflatable section 210 may be longitudinally asymmetric with a smaller tapering angle μ at the proximal tapering section 212 than the tapering angle β at the distal tapering section 214 of the balloon 200, which may help facilitate access into tight lesions. In other embodiments, the tapering angles μ and β may be substantially the same. In addition, embodiments of the distal cylindrical section 206 of the balloon 200 comprise a smaller outer diameter at OD3 than the proximal cylindrical section's 202 outer diameter, which may also facilitate access into tight lesions. Stated differently, the crossing profile of the device distal of the substantially cylindrical section 216 of the balloon 200 is smaller than the crossing profile of the device proximal of the substantially cylindrical section 216 of the balloon 200.

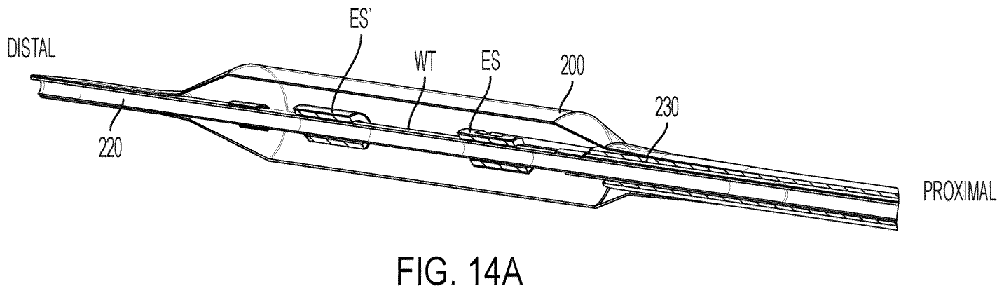

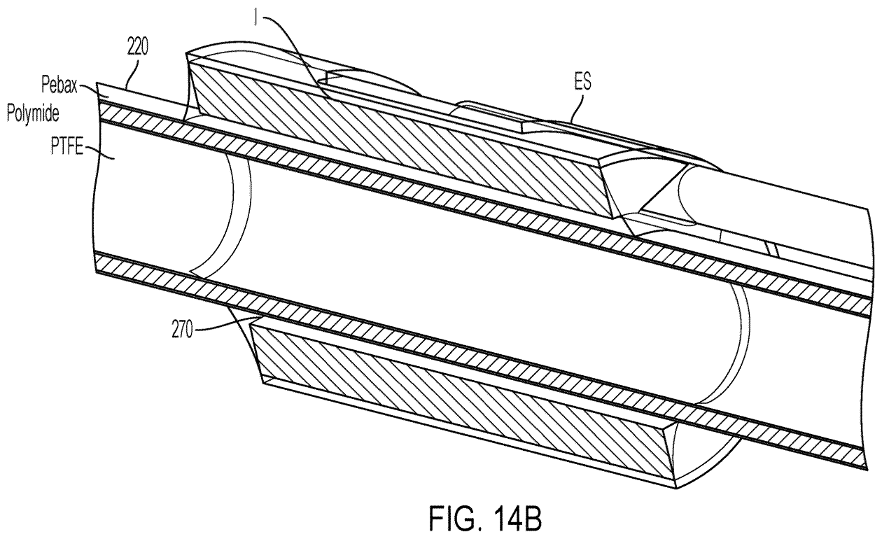

As shown in FIGS. 7B and 14B, the elongate member 220 to which the balloon 200 is sealed on the distal side comprises a polyimide core, which is lined on an inner surface with polytetrafluorethylene, commonly known as PTFE. The outer surface of the polyimide core is lined with 72D Pebax®. That said, other polymers or metals, or combinations or modifications thereof can be used for the elongate member 220. The electrode support members ES (proximal), ES' (distal) may be stainless steel and are coated with an insulating material such as a polymer or a blend of polymers or other materials, including but not limited to an adhesive, polyimide or other high temperature resistant flowable non-conductive material. That said, other metals or alloys can be used for the electrode support members (ES, ES′).

Exemplary dimensions for the balloon 200 region may comprise the length of extension of the distal end of the outer member 230 into the inflatable section 210 a distance. An exemplary distance of extension of the outer member's distal end into the inflatable section is 0.794 mm, but other extension distances are within the scope of the present disclosure.

The balloon wall may have a varying nominal thickness therethrough, e.g., longitudinally, circumferentially, or a combination thereof. For example, on the proximal side 204 and the distal side 206 of the balloon may have a thicker wall than a thickness of the wall in the middle portion of the balloon. Testing was performed using balloons constructed of a single layer of Vestamid ML21 having a double-wall thickness 222 in a mid-region (e.g., the substantially cylindrical portion 216) of the balloon 200, e.g., adjacent electrodes, of 0.0018″+/−0.0005″ (providing a nominal wall thickness of about 0.0009″ in that region). The nominal wall thickness at a proximal portion 204 of the balloon was within the range from 0.0012″ to 0.002″. As used in the art, a “double-wall thickness” means twice a nominal wall thickness and can be measured non-destructively on a folded balloon by observing a thickness of the folded balloon, for example, by radially collapsing a balloon to bring two opposed layers together and measuring the resulting thickness of the two opposed layers.

The balloon may be comprised of nylon or similar material. In some embodiments, the balloon material is uncoated, which may allow for more efficient transfer of energy from the pressure waves therethrough. Alternatively, the balloon can be coated with a hydrophilic coating, a drug coating, or any combinations or modifications thereof. In still other configurations, the balloon can be formed of a polymer, polyesters, thermoplastics, polyamides, modifications and combinations thereof. In still yet other configurations, the balloon may be made of a single material or composite layer of materials. Tensile strength may be optimized for durability purposes.

In addition, the presence of the tapering outer member 230 which adds stiffness to the device, the unbonded section 203 of the balloon 200, the tapering section 232 of the tapering outer member 230 which provides for a smaller crossing profile in a wrapped balloon configuration, all function to provide additional pushability and strength in the region of the outer member, and further works to prevent kinking of the wrapped balloon device during advancement through a patient's vasculature, both of which are highly advantageous.

As shown in FIGS. 6 and 9-11, a proximal marker band BP and a distal marker band BD may be provided within the balloon's inflatable portion 210 and disposed around the elongate member 220 at or near the transition from, respectively, the proximal and distal tapered sections 212, 214 into the substantially cylindrical section 216. In addition, a first proximal electrode support member ES is located along the elongate member 220 within the inflatable portion 210 at a position that may be closer to a proximal side of the balloon 200. In some embodiments, a single electrode support member ES may be provided as will be discussed further.

A second (distal) electrode support member ES' may be located along the elongate member 220 within the inflatable portion 210 at a position that is spaced distally from the first (proximal) electrode support member ES and closer to the distal side of the balloon 200. The two electrode support members ES and ES' are operatively and electrically connected by wire conductors W that are in operative electrical communication with the voltage pulse generator 10 as will be discussed further.

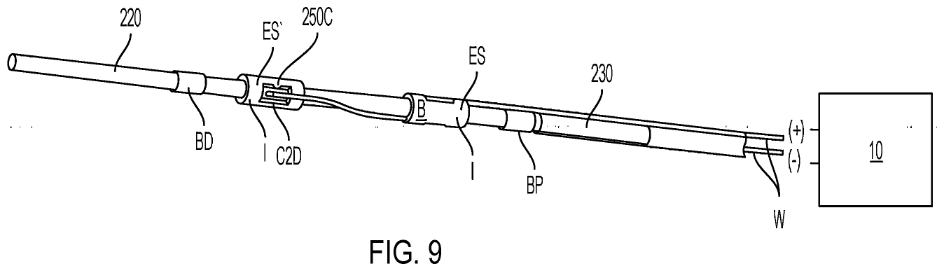

FIG. 9 illustrates a portion of the elongate member 220 and outer member 230 with the balloon 200 removed. Here, the first and second electrode support members ES, ES' are shown in closer detail with the wire conductors W connected in a series connection. Each electrode support member ES, ES' comprises a body B comprising a conductive material that is coated with an insulating material I and comprises at least one, preferably two, cutouts as will be discussed further. A tab or an arcuate region is defined on one of two longitudinal sides of each cutout and a wire conductor having an insulating covering with the exception of the distal-most end of the wire conductor which has no insulation As will be described further, a spark gap is formed between the exposed wire of the wire conductor and the tab or arcuate region of the cutouts of the electrode support members ES, ES′.

Each electrode support member may comprise two rotationally spaced cutouts with spark gaps which may be rotational spaced 180 degrees apart from each other, or may be spaced rotationally from each other at a different rotational spacing. As shown in FIG. 9, the spark gaps formed by the electrode support members ES and the spark gaps formed by the electrode support member ES' may also be rotationally spaced from each other. For example, if the spark gaps of the electrode support members ES and ES' are spaced 180 degrees apart around the respective electrode support member ES, ES′, then the two electrode support members ES and ES' may be radially rotated to ensure that all spark gaps are rotationally spaced apart from each other to provide circumferential coverage. In some embodiments, two or more cutouts and the respective spark gaps may be longitudinally aligned. As best shown in FIGS. 6 and 9, the electrode support members ES and ES' may be rotated relative to each other such that the respective cutouts and spark gaps are also rotationally spaced from each other around the elongate member 220. A preferred rotational spacing may comprise a 90 degree rotational spacing between spark gaps along the elongate member 220, though other rotational spacings are within the scope of this disclosure. It will be understood that in other configurations, the spark gaps of the electrode support members ES and ES' are spaced less than or equal to 360 degrees apart around the respective electrode support member ES, ES′, such as for example between about 0 degrees and about +10 degrees, between about 0 degrees and about +20 degrees, between about 0 degrees and about +30 degrees, between about 0 degrees and about +40 degrees, between about 0 degrees and about +50 degrees, between about 0 degrees and about +60 degrees, between about 0 degrees and about +70 degrees, between about 0 degrees and about +80 degrees, between about 0 degrees and about +90 degrees, between about 0 degrees and about +100 degrees, between about 0 degrees and about +110 degrees, between about 0 degrees and about +120 degrees, between about 0 degrees and about +130 degrees, between about 0 degrees and about #140 degrees, between about 0 degrees and about +150 degrees, between about 0 degrees and about +160 degrees, between about 0 degrees and about +170 degrees, between about 0 degrees and about +180 degrees, between about 0 degrees and about +10 degrees, between about +10 degrees and about +20 degrees, between about +20 degrees and about +30 degrees, between about +30 degrees and about +40 degrees, between about +40 degrees and about +50 degrees, between about +50 degrees and about +60 degrees, between about +60 degrees and about +70 degrees, between about +70 degrees and about +80 degrees, between about +80 degrees and about +90 degrees, between about +90 degrees and about +100 degrees, between about +100 degrees and about +110 degrees, between about +110 degrees and about +120 degrees, between about +120 degrees and about +130 degrees, between about +130 degrees and about +140 degrees, between about +140 degrees and about #150 degrees, between about #150 degrees and about +160 degrees, between about +160 degrees and about #170 degrees, or between about +170 degrees and about #180 degrees. Within the 360-degree rotational (or circumferential) spacing, there may be a singular event (e.g., arcing) location or a plurality of event locations. The temporal order of the events may follow the rotational spacing in an increasing order, decreasing order, or random order.

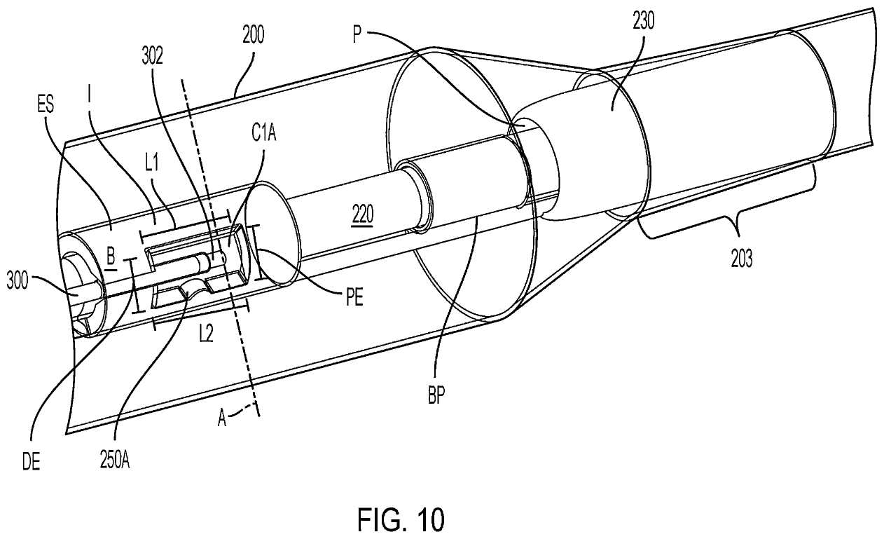

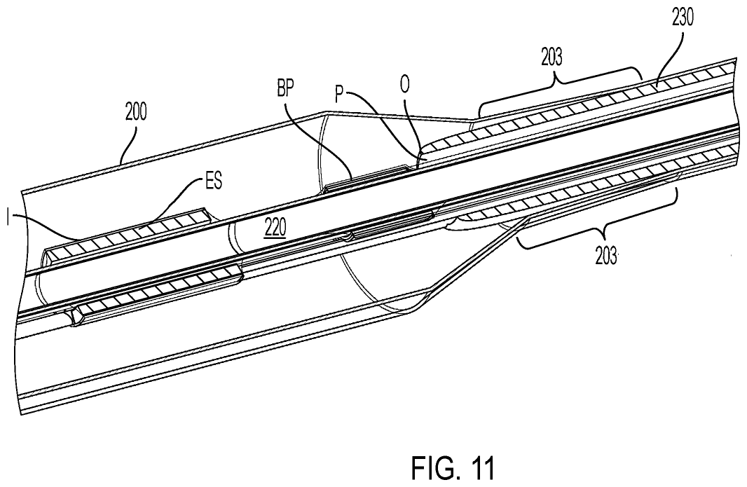

FIGS. 10 and 11 illustrate cutaway views of a proximal side of the balloon 200 and catheter structure, with a 1st (proximal) electrode support member ES shown with an associated wire conductor W. In addition, the wire conductors leading back to the positive and negative terminals of the voltage pulse generator occupy the fluid conveying pipe.

FIG. 10 also illustrates a proximal-most 1st electrode support member ES comprising a body B comprising a conductive material and defining a cutout C1A having two opposing longitudinal sides L1, L2 and opposing proximal and distal ends PE, DE. A tab or arcuate region 250 is formed or defined along one of the longitudinal sides L1. The surfaces of the electrode support body ES are covered in an insulating material I, with the exception of the tab or arcuate region 250, which comprises exposed conductive material.

As illustrated, a wire conductor 300 comprising insulation and having an exposed wire distal end region 302 that extends from a distal end a distance proximally. A proximal end region of the exposed wire region 302 is shown as located within the cutout, proximate to, and laterally or radially spaced from, the tab or arcuate region 250A. This configuration provides a pair of spaced-apart electrodes defining a spark gap between a lateral surface, and preferably not the distal end surface, of the exposed wire region 302 (comprising a first electrode in the illustrated spaced-apart pair of electrodes) and the tab or arcuate region 250 (comprising a second electrode in the spaced-apart pair of electrodes). In some embodiments, the distal end surface face of the wire conductor 300 may serve as an electrode in the above configuration.

The preferred embodiment comprises the lateral surface of the exposed wire conductor and that is located at a distal region of the wire conductor, serving as one of the electrodes in a spaced-apart electrode pair. The illustrated embodiment of FIG. 10 comprises the lateral surface of the exposed wire region 302 of the wire conductor as a first electrode, meaning that current flows to this electrode first, then across the spark gap to the second electrode of the spaced-apart pair of electrodes. As will be discussed, this current flow may be reversed in certain embodiments of the spaced-apart electrodes, wherein the metallic region (in the FIG. 10, the embodiment is a tab or arcuate region 250A) comprises the first electrode in the spaced-apart electrode pair. In this embodiment, the lateral surface of the wire conductor comprises the second electrode in the pair of spaced-apart electrodes and current flows to the first exemplary tab or arcuate region 250A, then across the spark gap to the second electrode comprising the lateral surface of the exposed wire region 302.

A preferred embodiment comprises the surface area of a first electrode and of a second electrode of a spaced-apart electrode pair to be substantially equivalent. In other embodiments, the surface area of a second electrode in a spaced-apart electrode pair may be larger than the surface area of a first electrode. Alternatively, the surface area of a first electrode in a spaced-apart electrode pair may be larger than the surface area of a second electrode.

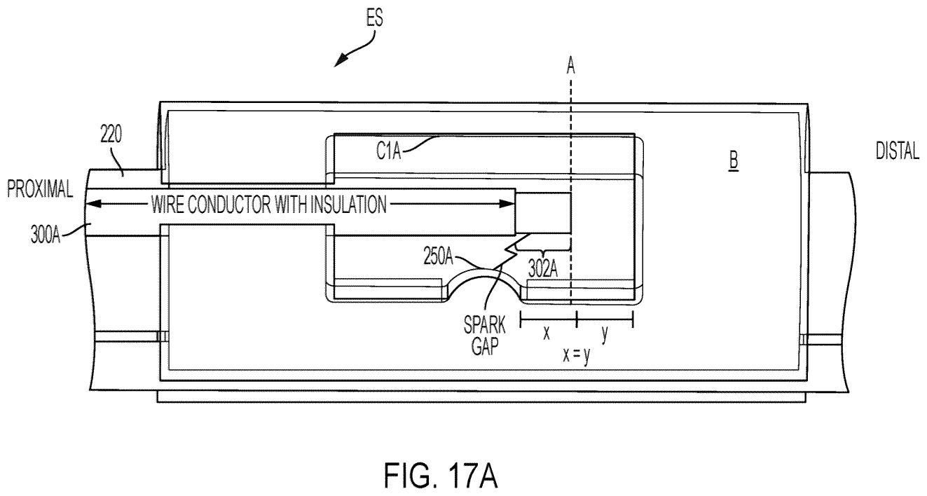

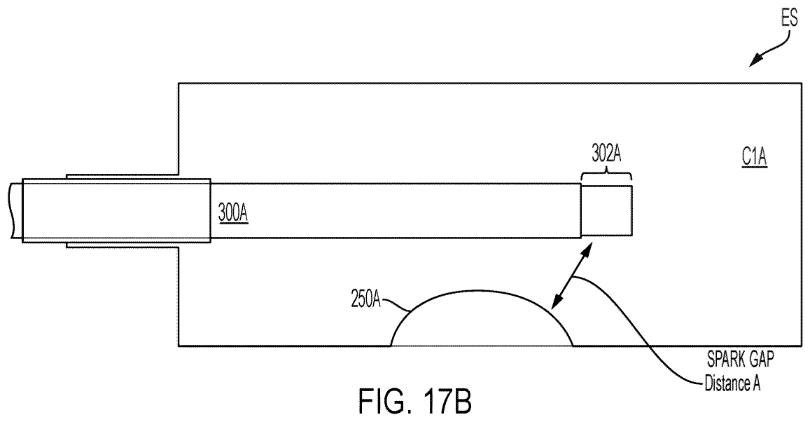

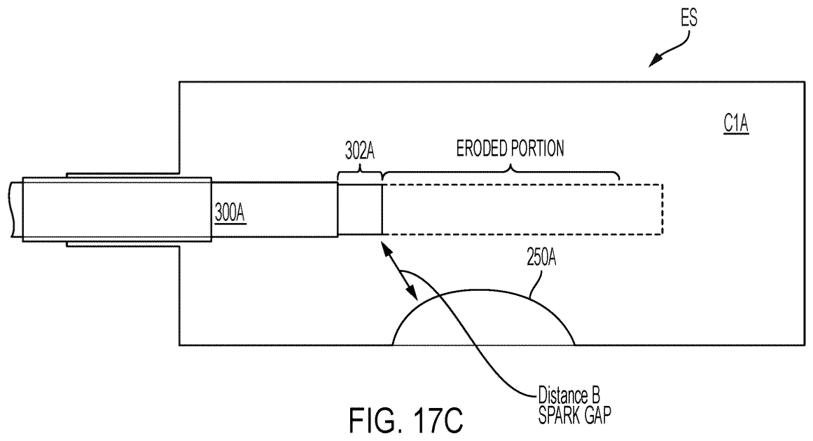

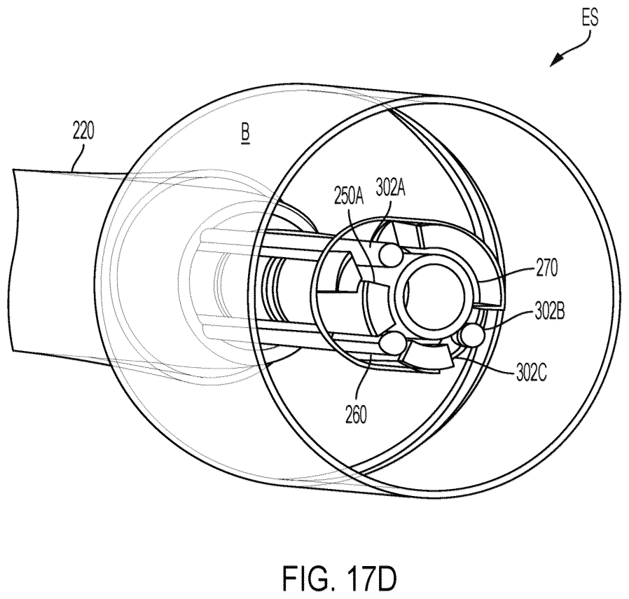

As will be discussed further, a preferred location for the exposed wire region 302 is to position the distal end approximately one half of the distance between the proximal end of the tab or arcuate region 250A and the proximal end PE of the cutout C1A. That location is shown by axis A in FIGS. 10, 17A-C, 18 and 19. In this configuration, the portion of the wire conductor directly overlaying the tab or arcuate region 250 remains insulated, with the lateral surface of the exposed wire region 302 being positioned beyond (in this case proximally beyond) the bounds of the tab or arcuate region 250. The spaced-apart electrodes of the embodiments described herein are preferably located along a common radial or circumferential plane.

Though the tab or arcuate region (2nd electrode) 250A is illustrated as positioned substantially mid-way along the length of the longitudinal side L1 of the cutout CIA, the tab or arcuate region 250A may be positioned more proximally or more distally as well. This changes the longitudinal position of the spaced-apart electrodes 250A, 302 as well as that of the defined spark gap between the spaced-apart electrodes 250A, 302, as well as effectively shifts the location and focus of the resultant pressure wave in the longitudinal direction to allow more effective coverage and/or interaction between adjacent generated pressure waves.

FIG. 11 illustrates a cutaway of the region illustrated in FIG. 10.

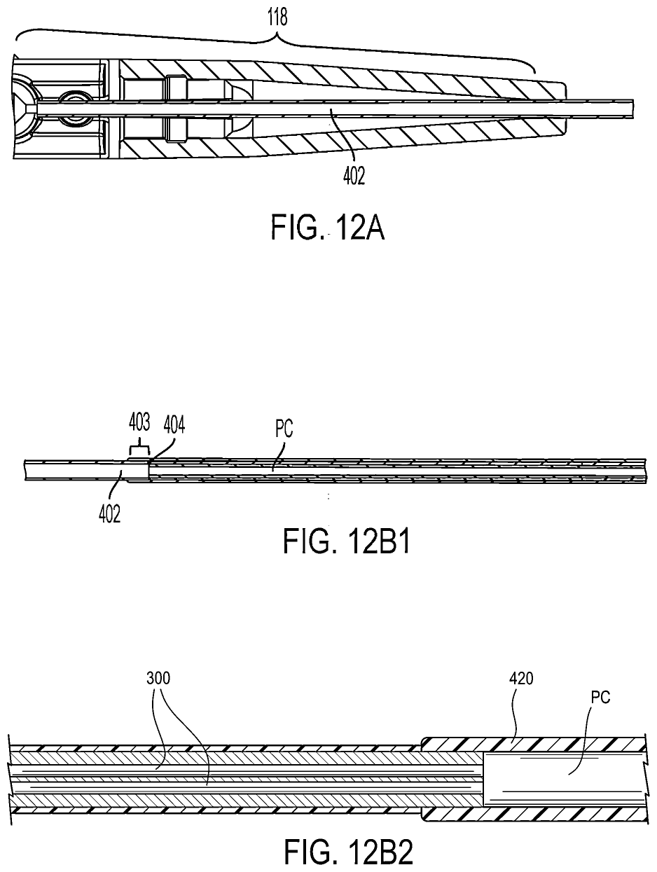

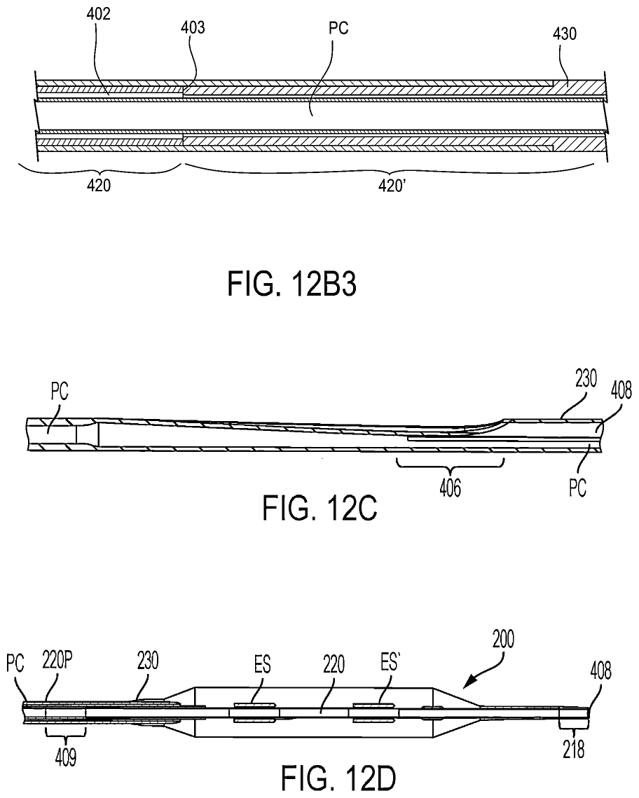

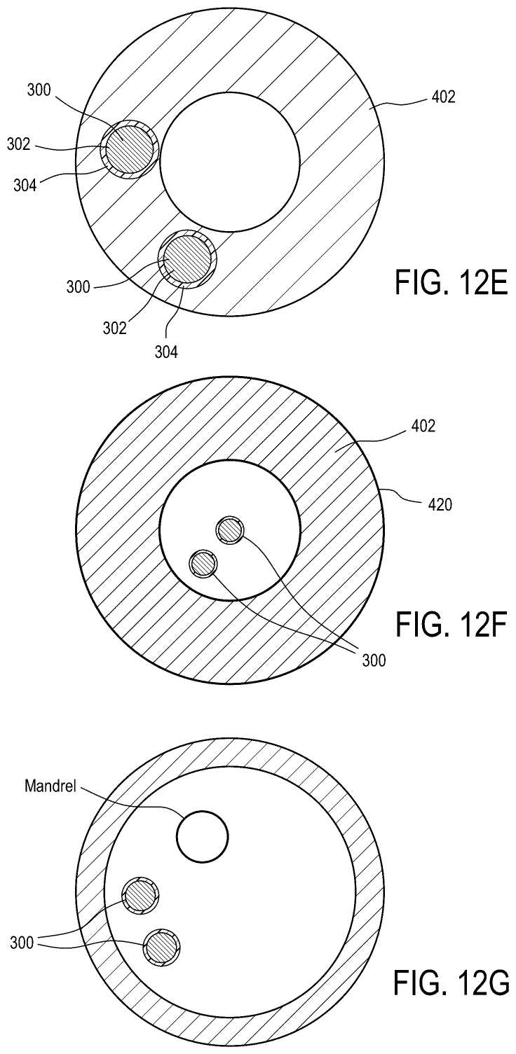

With continued reference to FIGS. 4-5 and 6-11, we now turn to FIGS. 12A-12D which illustrate features of an embodiment of an exemplary IVL system. FIGS. 12A-12D move successively distally along the exemplary system.

Beginning with FIG. 12A, just distal to the hub 118 as in FIG. 4, a hypotube 402 is provided which serves as a conduit for fluid infusion and removal and which is in fluid communication with the fluid reservoir and pump 114 as well as the interior of the inflatable portion 210 of the balloon 200. The hypotube 402 may be comprised of a metal and may be stainless steel. As shown in the combination of the structure shown in FIG. 12B, which is distal to the structure of FIG. 12A, an exemplary length of the hypotube 402 may be slightly longer than 1055 mm, though other lengths are within the scope of the present disclosure. FIG. 12B1 indicates a length of 1055 mm, however the hypotube 402 extends a slight distance further in the distal direction, along which it is overmolded by a 72D Pebax® material to form a bonded section 403 in order to, among other things, add bonding strength. The hypotube 402 is generally not coated on an outer or inner surface, but comprises a polymer near the distal end, e.g., Pebax® for aid in joining or bonding, and an adhesive on the proximal end region, also for aid in joining or bonding.

The hypotube 402 is shown as terminating distally at a distal tip 404 in FIG. 12B1. In some embodiments, the hypotube 402 may extend from the hub distally a distance of about 1080 mm. FIG. 12B1 continues a distance (e.g., and without limitation approximately 280 mm) distally from the bonded section 403 to comprise a polyimide conduit PC for strain relief which is coated on its outer surface with the 72D Pebax®, which also comprises the bonded section 403. The hypotube 402 is shown as terminating distally at the distal tip 404 in FIGS. 12B1 and 12B3 and the bonded section 403 is overlapped with an overmolded polymer material such as a Pebax®, for example 72D Pebax® surrounding a distal region of the hypotube 402.

FIGS. 12A-12C illustrate features of the catheter shaft of FIGS. 4 and 5 and FIG. 12A illustrates a cutaway view of one embodiment of the hub 118 as discussed in relation to FIGS. 4 and 5. A hypotube 402 is disposed through at least a portion of the hub 118 and which may serve as, inter alia, a conduit for fluid infusion and removal and, therefore, is in fluid communication with the fluid reservoir and pump 114 as well as the interior of the inflatable portion 210 of the balloon 200. In addition, the hypotube 402 may comprise a metal and may be stainless steel, for example. As a result, the hypotube 402 can provide strong columnar strength and pushability characteristics to the catheter. The hypotube 402 is shown as terminating distally at 404 in FIG. 6 and comprises an overlapping bonded section 403 with an overmolded polymer materials, such as, for example, a Pebax®, for example 72D Pebax® surrounding a distal region of the hypotube 202.

In addition to the hypotube 402 surrounded by an overmolded polymer material, the overlapping bonded section 403 also comprises a polyimide tube or conduit PC located within a lumen formed or defined by the hypotube 402. The polyimide tube or conduit PC acts, inter alia, as a strain relief that allows maximization of columnar strength which, in turn, translates into increased transfer of axial push force, or pushability, of prior systems while reducing or minimizing kinking.

FIG. 12B2 further illustrates the overlapping bonded section 403 with the exemplary stainless steel hypotube 402, which is initially surrounded by a thin layer of an outer wall polymer 420, which is made of exemplary 72D Pebax® polymer which continues distally, providing an outer layer for at least a length of the catheter. The polyimide tube or conduit PC extends in a distal direction away from a distal end portion of the hypotube 402. The exemplary 72D Pebax® polymer 420 extends from the proximal portion of the bonded section 403 to the distal portion of the bonded section 403. Although reference is made to the wall polymer 420 being formed of 72D Pebax® polymer, it will be understood that other materials, polymers, etc. are possible for forming the outer wall polymer 420.

FIG. 12B3 provides a close-up view of the distal end portion of the hypotube 402 and the transition to the polyimide tube or conduit PC. As illustrated, the exemplary 72D Pebax® polymer 420′ comprises a relatively thin wall in the overlapping bonded section 403. However, as will be discussed further below, the 72D Pebax® wall thickness may increase distal to the distal end 404 of the hypotube 402. This wall thickness differential and transition allows for a balance to be achieved between a highly desirable small crossing profile in the overlapping bonded section 403 with retention of sufficient columnar strength and resulting pushability for the distal end of the catheter shaft. For instance, since the strength at a given longitudinal position of the shaft need only push the distal remainder of the catheter, as one approaches the distal end of the catheter, the thickness can decrease since there's less longitudinal resistance to overcome, and the decreased thickness and smaller crossing profile at the distal portion of the catheter help enhance trackability of the catheter.

FIGS. 12B2 and 12B3 further illustrate the wall thickness transition of the exemplary 72D Pebax® polymer 420′ forming an outer wall for the catheter shaft. Both FIGS. 12B2 and 12B3 illustrate the hypotube 402 with the polyimide tube or conduit PC extending distally from the distal tip 404 of the hypotube 402, surrounded by the outer wall polymer 420 and 420′.

FIG. 12B3 provides a close-up view of the distal end 404 of the hypotube 402 and the relatively thin outer wall or layer of polymer 420 surrounding the hypotube 402, followed distally by a relatively thick outer wall of polymer 420′ surrounding the polyimide tube or conduit PC.

The thickness of polymer layer 420′ relative to wall thickness of polymer layer 420 may be formulated as follows: In some embodiments, the polymer layer 420′ may be between 10% and 50% thicker than the thickness of polymer layer 420. In some embodiments, the polymer layer 420′ may be between 50% and 60% thicker than the thickness of polymer layer 420. In some embodiments, the polymer layer 420′ may be between 60% and 70% thicker than the thickness of polymer layer 420. In some embodiments, the polymer layer 420′ may be greater than 70% thicker than the thickness of polymer layer 420.

FIG. 12E shows a cross-section of the catheter shaft proximal to the overlapping bonded section 403 with conductors or wires located within a wall of the catheter shaft, such as within a portion of the hypotube or a polymer tube. FIG. 12F shows a cross-section of the catheter shaft within the overlapping bonded section 403 and comprising the outer polymer layer 420, such as 72D Pebax® coating in one configuration, surrounding the hypotube which surrounds the polyimide tube or conduit. Wire conductors 300 are also exemplified within the overlapping bonded section 403.

The resulting outer diameter within the overlapping bonded section 403 of the present disclosure is less than about 0.044 inches. More preferably, the outer diameter of the overlapping bonded section 403 is less than about 0.041 inches. Still more preferably, the outer diameter of the overlapping bonded section 403 is equal to or less than 0.0405 inches. In contrast, a known IVL device comprises an overlapping section including a hypotube located along an IVL catheter shaft with an outer diameter of about 0.0435 inches. The significant reduction in outer diameter, and crossing profile, of 0.003 inches in the presently described IVL catheter, as compared with the known catheter device, is primarily driven by the reduced polymer wall thickness in the region of the overlapping bonded section 403 as described above. The result is that the presently described IVL catheter comprises a maximum outer diameter that is significantly less than the maximum outer diameter of the known IVL catheter device. Stated differently, the presently described IVL catheter comprises a maximum crossing profile that is significantly less than the maximum crossing profile of the known IVL catheter device.

Some embodiments of the presently disclosed IVL catheter 100 may comprise a polymer wall 420′, e.g., 72D Pebax®, extending distally away from the hypotube's distal end with a wall thickness of about 0.009 inches beginning with the thickened polymer wall 420′ as shown in FIGS. 12B1-12F. Some embodiments may comprise the thickened polymer wall 420′ beginning with a thickness of about 0.009 inches and tapering distally to a wall thickness of about 0.0038 inches at a point just proximal to the balloon or enclosure location.

FIG. 12G illustrates a known IVL catheter that includes an internal, tapered support mandrel or tube to provide pushability to the system. This support mandrel runs from distally from a mid-catheter position at the overlapping section at a distal end of the known device's hypotube, as discussed above, to the known device's IVL balloon. This support mandrel floats within the lumen of the known IVL catheter for most of the support mandrel's length. The proximal portion of the mandrel support is thicker than distal portions, adding stiffness to the overall catheter in the proximal region with reduced stiffness moving in the tapering and distal direction.

With continued reference to FIGS. 7-9B, FIG. 13 illustrates a mid-catheter portion of the shaft of the IVL catheter 100 discussed above. This cross-sectional image is located at a point that is distal to the overlapping bonded section 403 discussed above and further distal to the distal end of the hypotube 202. As shown, the shaft comprises conductive wires located within the shaft, but does not require a support mandrel in contrast with the known IVL catheter of FIG. 12. The absence of a support mandrel in the mid-catheter region, distal to the hypotube 202, in combination with the thickened wall 206′ of FIG. 9B, enhances deliverability of the IVL catheter 100. The thickened wall 206′ also provides a uniform kink resistance in all bending or flexing directions. This is also in contrast with the known IVL catheter's structure as the support mandrel floats within the catheter, so that kink resistance will be dependent upon the location of the floating support mandrel within the catheter at any given point in time.

Additionally, as illustrated in FIG. 12B2, within the hypotube 402, a pair of electrodes are located and saline solution passes along the pair of electrodes. The starting position of the outer wall polymer 420 is proximal to the proximal position of the banded section 403. As illustrated in FIG. 12B3, the ending position of the outer wall polymer 420′ is distal to the banded section 403. In other words, the outer wall polymer 420 surrounds an area, which covers more than the bonded section 403 in both directions along the longitudinal axis of the catheter.

Even though the outer wall polymer is identified as “420” from the proximal portion of the banded section 403 to the distal tip 404 and separately as “420” from the distal tip 404 of the hypotube 202, as illustrated in FIG. 12B3, the outer wall polymers 420 and 420′ are the same outer wall polymer but separately identified to clearly show two portions of the outer wall polymer around the distal tip 404 of the hypotube 402. The thickness of the outer wall polymer 420′ relative to wall thickness of the outer wall polymer 420 may be formulated as follows. In some embodiments, the outer wall polymer 420′ may be between 10% and 50% thicker than the thickness of the outer wall polymer 420. In some embodiments, the outer wall polymer 420′ may be between 50% and 60% thicker than the thickness of the outer wall polymer 420. In some embodiments, the outer wall polymer 420′ may be between 60% and 70% thicker than the thickness of the outer wall polymer 420. In some embodiments, the outer wall polymer 420′ may be greater than 70% thicker than the thickness of the outer wall polymer 420.

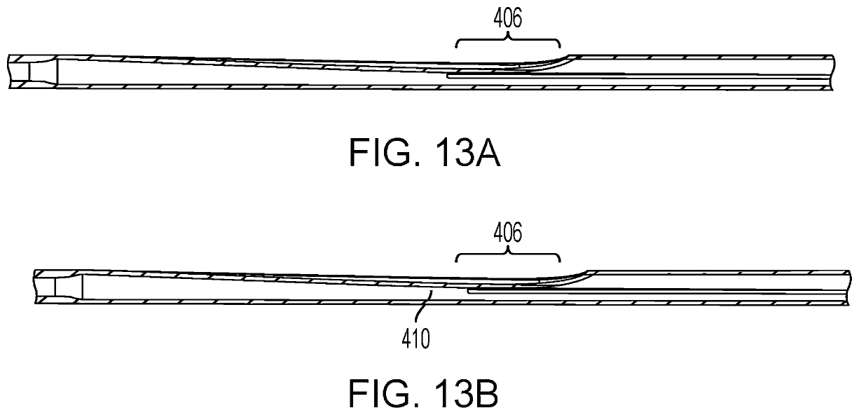

FIG. 12C illustrates the section comprising an RX port 406, which provides access for a guidewire or other interventional tools. The RX port 406 leads to a guidewire conduit 408 which may comprise a 63D Pebax® tube with a high density polyethylene (HDPE) such as Rezilok and having an inner surface that may be lined with 63D Pebax®. The guidewire conduit 408 extends distally through the polyimide conduit PC and the elongate member 220 and leads out of the system at the distal end of the distal tip 218. The RX port 406 is discussed further in FIGS. 13A and 13B.

A proximal end of the outer member 230 comprising 63D Pebax® that is lined with HDPE, e.g., Rezilok, is provided just distal to the RX port 406 as shown in FIG. 12C. The outer member 230 continues for a length or distance distally to terminate at a distal end that is located within the inflatable section 216 of the balloon as shown and described above, as well as illustrated in FIG. 12D. The elongate member 220 comprises a proximal end 220P that connects with the polyimide conduit PC described above and comprising a bonded, e.g., laser reflowed, section 409 to aid in bonding. The elongate member 220 extends through the outer member 230 and the interior of the balloon 200 to a point that is just proximal to the distal tip 218.

Turning now to FIGS. 13A and 13B, the RX port 406 is illustrated. As shown a hypotube 410 is provided, which may comprise a polymer, along a length of the polyimide tube on the proximal side of the RX port 406 for support. With supplemental reference to FIGS. 14A and 14B discussed below, known devices typically employ a support wire instead of the hypotube 410, but the inventors found that the polyimide tube with an outer polymer jacket comprising the elongate member 220 that transitions into the hypotube 410 solution provides greater and necessary stiffness and support in this critical region.

FIG. 14A illustrates a cross-sectional view of the tapering outer member 230, balloon 200, elongate member 220 with 1st and 2nd electrode support members ES, ES′. Electrode support members are in operative electrical communication with a voltage pulse generator 110 as discussed above. A bridge wire WT is provided for operative electrical connection between ES and ES′. The bridge wire is preferred to be made of tantalum rather than the known IVL devices which use copper wire throughout. Tantalum provides significantly enhanced durability as compared with copper. During testing, copper wire used as a “bridge wire” would slowly deteriorate or erode as the test voltage pulses and associated electrical arcs and current flow progressed. Ultimately, the copper bridge wire became susceptible to becoming displaced and interrupting the series connection between electrode support members ES and ES′. Thus, tantalum bridge wires were found to provide key durability characteristics, one of the features of the present disclosure that allows for a far greater number of electrical areas (up to, and greater than, 500) to be produced than the known devices.

FIG. 14B is a closeup of a cross-section of an electrode support member of FIG. 14A. The elongate member 220 may comprise 3 layers: a core of polyimide which aids in resistance of heat generated by the electrodes during operation, an outer layer of Pebax and an inner layer of PTFE. Other polymers or blends of polymers may be used in the construction of the elongate member 220.

In some embodiments, an air or fluid gap 270 is provided between the electrode support member, for example ES and/or ES′, and the outer surface of the elongate member 220 to which the electrode support member ES and/or ES' is adhered or operatively connected and at least partially surrounds. The air or fluid gap 270 functions to help to dissipate heat generated by the electrical arcs produced across the spark gap between the two spaced-apart electrodes 250, 302 described above, allowing the fluid within the inflated balloon 200 to flow through the air or fluid gap 270, and around and underneath a portion of the electrode support member ES and/or ES' to remove heat from the structure. This is another key durability element of the present disclosure and contributes to the far greater number of electrical arcs, and at a greater frequency of electrical arcs, that are produced with embodiments of this disclosure compared with the known devices.

Further, the electrode support members, e.g., ES, ES′, are coated with an insulating material, with the exception of the bare metal electrode element, e.g., tab or arcuate region 250, and the wire conductors defining an exposed wire electrode element are also otherwise coated with an insulating material. As a result, the surface area of the spaced-apart electrodes in each case is relatively tightly controlled. This is in contrast to a known IVL system which provides concentric metallic electrodes with much more exposed metal surface area than is actually required and, as a result, produce much more undesirable gas as a by-product of generating electrical arcs. This is another key feature of the present disclosure that contributes to durability as well as improved variability when compared with known IVL systems.

The operational connection or adherence of the electrode support members ES and/or ES' with the elongate member 220 in this embodiment is unique in that, as discussed above, the electrode support member ES and/or ES' is coated or covered with an insulating material I. The insulating material I flows beneath portions of the electrode support member ES and or ES' to form a connection or adherence between portions of the lower surface of the electrode support member ES and/or ES' and the outer surface of the elongate member 220, while retaining the desired air or fluid gap 270.

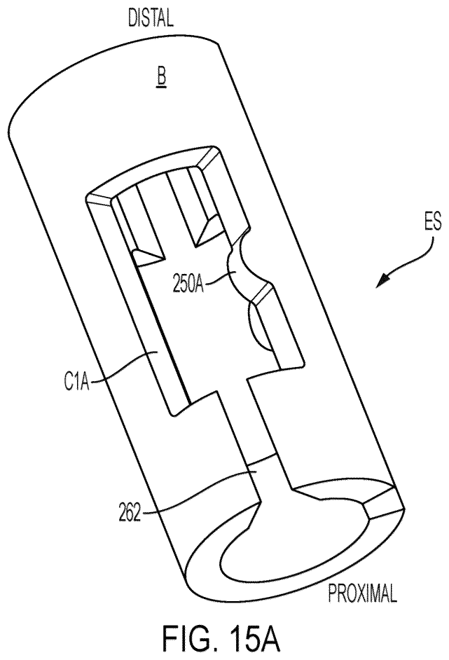

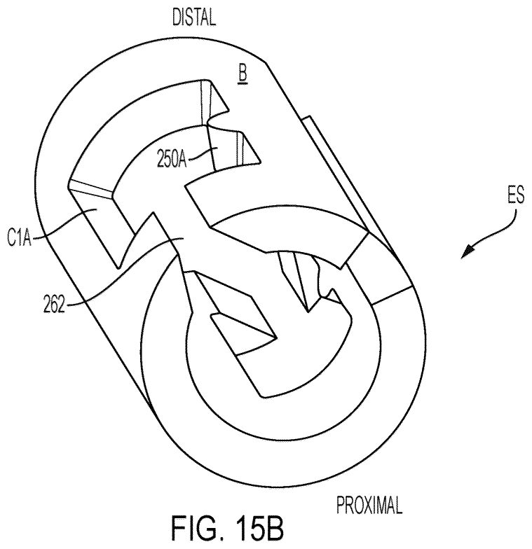

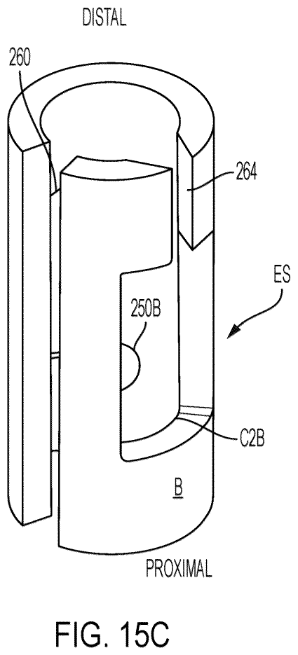

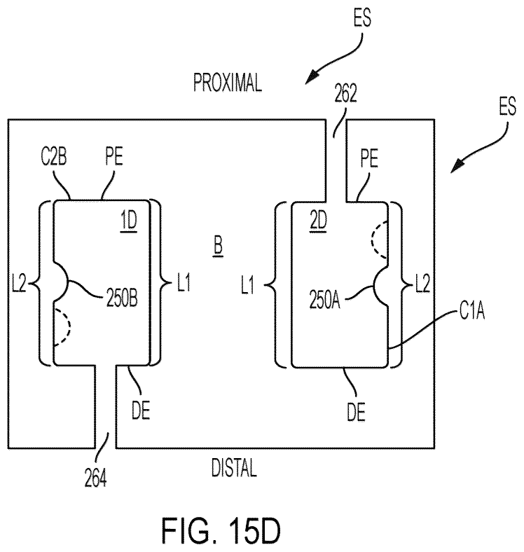

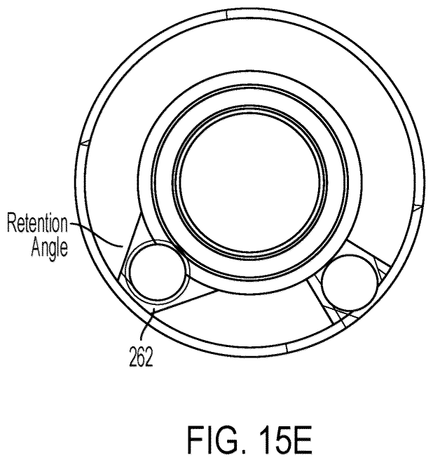

Turning now to FIGS. 15A-15E, one embodiment of an electrode support member ES comprising two electrodes is illustrated. The electrode support member ES may be used in the IVL system embodiments described above. The electrode support member ES comprises a body B which may be cylindrical and configured to at least partially surround the elongate member 220 as discussed above. In some embodiments the electrode support members discussed herein may not be fully circumferential as will be discussed further. The embodiment of FIGS. 15A-D comprises two radially spaced-apart cutouts, a first cutout C1A and a second cutout C2B. Each cutout C1A, C2B comprises opposing longitudinal sides L1, L2 and a proximal end PE and a distal end DE. The body B also comprises a longitudinally arranged slot or channel 260 that extends all the way along the body and configured to receive a portion of an insulated wire conductor. The first cutout C1A comprises a slot or channel 262 extending longitudinally away in a proximal direction from the proximal end PE of the first cutout C1A. The second cutout C2B comprises a slot or channel 264 that extends longitudinally away in a distal direction from the distal end DE of the second cutout C2B.

The electrode support member ES comprises a body formed of a conductive material which is covered with an insulating material I as described above. A region along one of the opposing longitudinal sides comprises exposed conductive material, with the insulating material covering removed. In the illustrated embodiment of ES, the exposed conductive material, e.g., metal, is provided along longitudinal sides L2 at 250A and 250B, respectively, for each the first and second cutouts CIA, C2B. The embodiment of FIGS. 15A-15E provides the exposed conductive material portion as individual arcuate regions 250A, 250B that extend radially into each cutout C1A and C2B, respectively. Each of the exemplary arcuate regions 250A, 250B defines one electrode of a spaced-apart electrode pair.

As best seen in FIG. 15C, the longitudinal slot or channel 260 running the entire length of the electrode support member body B is provided and is configured to receive insulated portion of one or more wire conductors. FIG. 15E illustrates a cross-sectional view through body B showing that the longitudinal slot or channel 262 may comprise angled sides, with a smaller opening at the outermost portion of the slot or channel 262. This angled retention structure may be used to retain wires in slots 260, 262 and/or 264.

The slots or channels 260, 262, 264 are provided to maintain a crossing profile of the electrode support members that is, at maximum, the outer diameter of the electrode support member body. In addition, the slots or channels 260, 262, 264 function to retain the wire conductors and associated electrode regions in the proper position within the subject electrode support member.

FIG. 15D provides an “unrolled” flattened view of the exemplary electrode support member body B. The electrode-defining arcuate regions 250A, 250B of exposed conductive material may be substantially centered along the longitudinal side L1 or L2 defining the arcuate regions 250A, 250B. Alternatively, as shown by the dashed lines, one or both of the arcuate regions 250A, 250B of exposed conductive material may be shifted away from the center of the longitudinal side. Thus, the arcuate regions 250A, 250B may be centered along the subject longitudinal side and/or shifted away from the center. In one embodiment, one of the arcuate regions, e.g., 250A may be offset longitudinally from the location of the other arcuate regions, e.g., 250B. This allows the locations of the arcuate regions 250A and 250B that are defined by a single electrode support member body B (and the resulting spaced-apart electrode, and defined spark gap locations) to be tuned and, in some embodiments, offset both radially and longitudinally from each other. This, in turn, allows for generation of pressure waves by a single electrode support body B with two radially spaced-apart electrode pairs, wherein the pressure waves produce mechanical forces that are not only offset radially from each other, but also offset longitudinally from each other.

The formation of the spaced-apart electrode pairs, and defined spark gaps, using exemplary embodiment ES are generally described above, and will be further discussed below.

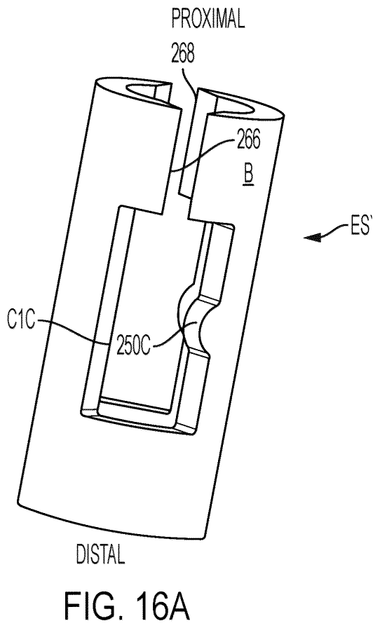

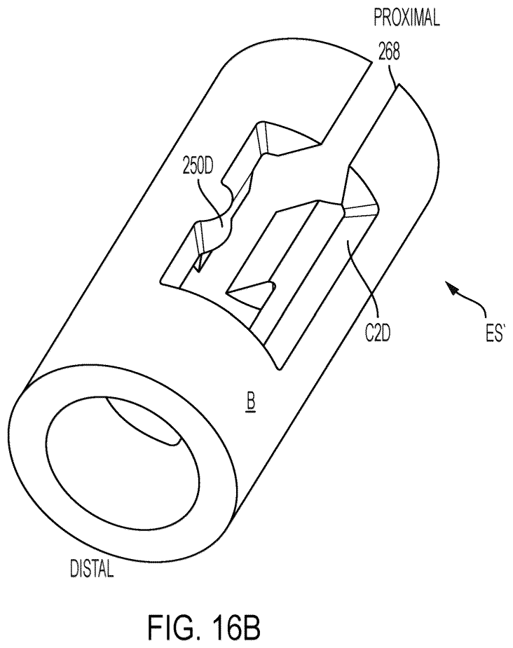

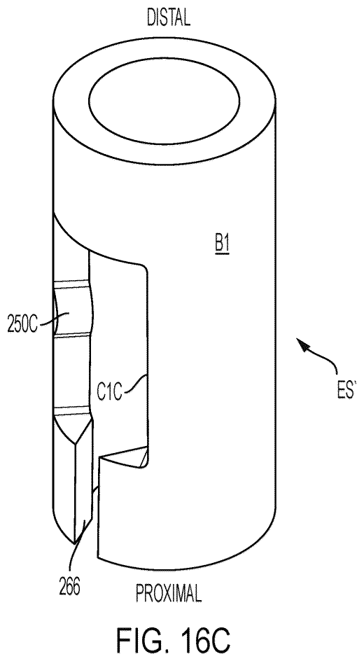

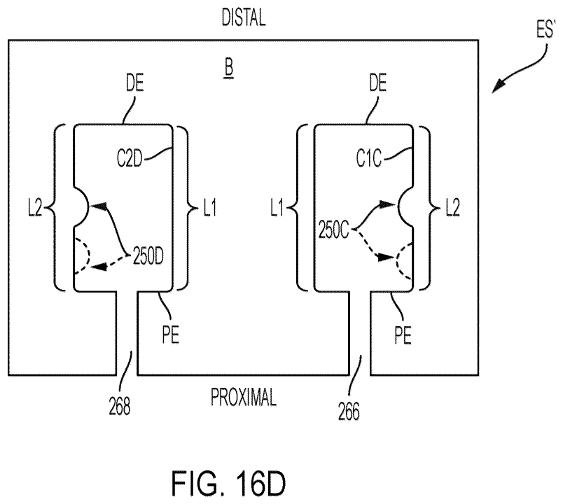

The exemplary electrode support member ES of FIGS. 15A-15E may comprise a proximal electrode support member such as shown in, e.g., FIG. 6 that is coupled with a more distally spaced-apart electrode support member ES' which is discussed below in FIGS. 16A-16D. In addition, when two or more of the electrode support members ES may be electrically and operatively connected with each other and with a more distally spaced-apart electrode support assembly, one of the electrode support members ES may comprise a proximal-most electrode support member and the remaining electrode support members ES may comprise intermediate electrode support members located between a proximal and a distal electrode support member.

A distal electrode support member ES' embodiment is illustrated in FIGS. 16A-16D. This embodiment may comprise a more distally located exemplary electrode support member ES′, such as shown in FIG. 6, when operatively combined with at least one more proximally-spaced apart electrode support member such as ES described above. More fundamentally and alternatively, this embodiment may be used alone, thus providing a single electrode support member ES' with two radially spaced-apart electrode pairs when fully assembled. In an alternate embodiment, the single electrode support member ES' may comprise a single pair of spaced-apart electrodes, wherein the spaced apart electrodes are in operative electrical communication with a first wire conductor that is in operative electrical communication with a first electrode of the electrode pair and a positive or high side terminal of a voltage pulse generator. A second wire conductor may be in operative electrical communication with a second electrode of the electrode pair and a ground or low side terminal of the voltage pulse generator.

The embodiment of FIGS. 16A-16D also comprises two radially spaced-apart cutouts, a first cutout C1C and a second cutout C2D. Each cutout C1C and C2D defines two opposing longitudinal sides L1, L2 and a proximal end PE and a distal end DE as well as a slot or channel 266, 268 extending in the proximal direction from the proximal end PE of each of the first and second cutouts C1C and C2D. As with the embodiment of FIGS. 15A-15E, exemplary arcuate regions of exposed conductive material 250C and 250D are defined along one of the longitudinal sides of cutouts C1C and C2D, respectively. As best seen in FIG. 16D, the regions of exposed conductive material, e.g., metal, 250C and 250D are each defined along longitudinal side L2 of the respective cutout C1C and C2D. Each arcuate region 250C and 250D forms and defines one electrode of a space-apart pair of electrodes. As with the embodiment of FIGS. 15A-15E, the location of one or both of the arcuate regions 250C and 250D (and the defined spark gap when the wire conductors are added, and the location of the pressure wave produced by the resultant spaced-apart electrodes) may be longitudinally shifted from the center of the defining longitudinal side(s) as shown with the dashed lines in FIG. 16D. This embodiment may not comprise the full length longitudinal slot 260 of the embodiment of FIGS. 15A-15E.

An exemplary spaced-apart electrode pair is illustrated in FIGS. 17A-17D. An exemplary electrode support member, which may be either of the embodiments discussed above in FIGS. 15A-16D, is provided. We will describe the illustrated embodiment as an electrode support member ES as described above in connection with FIGS. 14A-14D. A first cutout C1A is shown illustrated with a portion of an insulated wire conductor 300A located or received within the slot or channel 262 extending proximally away from the first cutout C1A. A distal-most region 302A of the wire conductor 300A is stripped of insulation, leaving an exposed distal-most region 302A of exposed conductive wire. Together with the exposed metal of the arcuate region 250A, the lateral surface, as opposed to the distal end face or surface, of the exposed distal most region 302a of the wire conductor 300A forms a spaced-apart electrode pair, with a spark gap defined therebetween.

The location of the exposed conductive wire region 302A is preferably positioned beyond the arcuate region as shown such that a distal end of the exposed wire region is halfway between the arcuate region and the distal end of the cutout C1A. This preferred dimensioning is illustrated by x and y, wherein x=y in FIG. 17A. Alternatively, the exposed wire region 302A at the distal end of the wire conductor may be positioned generally over the arcuate region to form an alternative embodiment of a spaced-apart electrode pair.

FIGS. 17B and 17C illustrate an exemplary starting and ending positions for the electrode comprising the exposed wire region 302A relative to the spaced-apart electrode comprising the exemplary arcuate region 250A. FIGS. 17B and C also illustrate a general direction of the current flow and electrical arc across the spark gap defined by the spaced-apart electrode pairs with distance A representing a starting spark gap length and distance B an ending spark gap length between the lateral face of the exposed wire conductor (exemplary first electrode) and the exposed conductive material of the arcuate region (exemplary second electrode). The current flow and resulting electrical arc will translate axially along with the translating exposed wire region 302A.

As the electrical arcing initiates and progresses between the spaced-apart electrodes 302A and 250A, the electrode comprising the exposed wire region 302A begins to erode and translate, effectively move axially (in the illustrative embodiment proximally) along the arcuate region 250A and engaging successively different (more proximal) regions of the arcuate region 250A in the electrical arcing process. The insulation initially covering the wire conductor 300A burns away, exposing successively more wire conductor and, as in FIG. 17C, with an exemplary ending position with a spark gap of distance B. As is now apparent, the spark gap changes relative locations, moving in this case in the proximal direction along a longitudinal axis of the wire conductor 300A. As the spark gap location changes, so does the effective direction of the current flow and resultant electrical arcs.

FIG. 17D illustrates a cross-sectional view through an exemplary electrode support member ES described in connection with FIGS. 15A-15E to show the relative positions and locations of exemplary first electrode comprising the exposed wire region 302A an exemplary second electrode comprising arcuate region 250A to define a first spaced-apart electrode pair. In addition, the relative positions and locations are shown for a second spaced-apart electrode pair, circumferentially or radially spaced away from the first spaced-apart electrode pair. The second spaced-apart electrode pair includes an exemplary second electrode comprising the exposed wire region 302B and an exemplary second electrode comprising arcuate region 250B. It is preferable that, in all embodiments described herein, the electrodes formed by the lateral face of an exemplary exposed conductive wire 302A, 302B and the respective and exemplary exposed metal region, illustrated as arcuate region 250A, B, be located at positions that are substantially the same relative to the outer surface of the elongate member 220. Stated differently, the spaced-apart electrodes formed by 302A, 250A and 302B, 250B may each be located a distance from the outer surface of the elongate member 220, wherein the distance from a spaced apart electrode 302A and 302B to the outer surface of the elongate member 220, and the distance from spaced-apart electrode 250A and 250B to the outer surface of the elongate member 220 are substantially equal. This arrangement provides an air or fluid gap 270 which is discussed further above. Moreover, with reference to FIGS. 15A-15E, at least part of the lateral surface of the exposed wire section of the first wire conductor may located between, and aligned with, the first and second longitudinal sides L1, L2 of the cutout such that the lateral surface of the exposed wire section is spaced apart from, and aligned with, the exposed metal region. Alternatively, the spaced-apart electrodes formed by 302A and 250A may each be located along the outer surface of the elongate member 220.

In all embodiments, the spaced-apart electrodes of the present disclosure are provided at substantially equal distances from a longitudinal axis through the IVL device's shaft.

In some embodiments, the spark gap represented by distances A and B may be of equivalent length. This is significant as it allows for use of a predictable and predetermined voltage magnitude to be provided which, in turn, produces a much more controlled pressure output from the produced pressure waves. Ultimately, the pressure output produced with a controlled, known spark gap length comprises more consistent, less variable forces than known IVL devices. This is understood to be critical in producing less strain on the balloon which, in turn, allows for a larger number of maximum voltage pulses, electrical arcs and produced pressure waves with a single catheter or system than is currently possible. For example, a known IVL coronary device has a maximum of 120 voltage pulses. The disclosed embodiment is demonstrated to effectively produce 300 voltage pulses per catheter in some embodiments and up to 500 pulses per catheter in some embodiments, with associated produced pressure waves, all within a very tight distribution and without significant decrease in the produced pressure outputs across the 300 voltage pulses. In some embodiments, the spark gap may be controlled such that it is within a predetermined range of length, with an exemplary minimum spark gap of 0.004″.

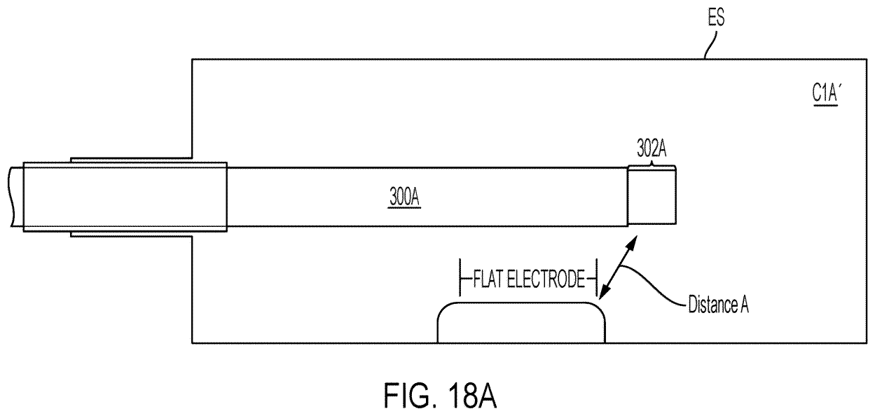

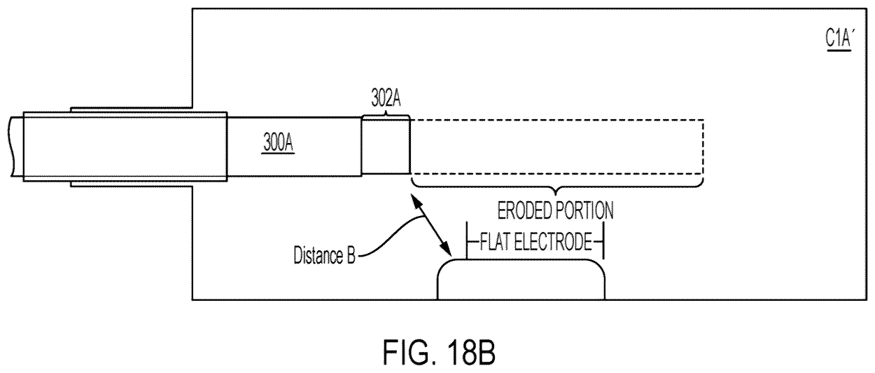

FIGS. 18A and 18B are similar to the embodiment of FIGS. 17A and 17B, wherein the arcuate region electrode is replaced with a cutout C1A′ comprising a raised flattened region that comprises exposed metal and functions as an electrode. This arrangement further ensures that, as the lateral surface of the exposed wire region 302A is involved in electrical arcs with the raised flattened region, the spark gap distance remains substantially the same across the executed series of voltage pulses and electrical arcs. Thus, distances A and B are substantially equal, and each spark gap therebetween is also substantially the same length as distances A and B.

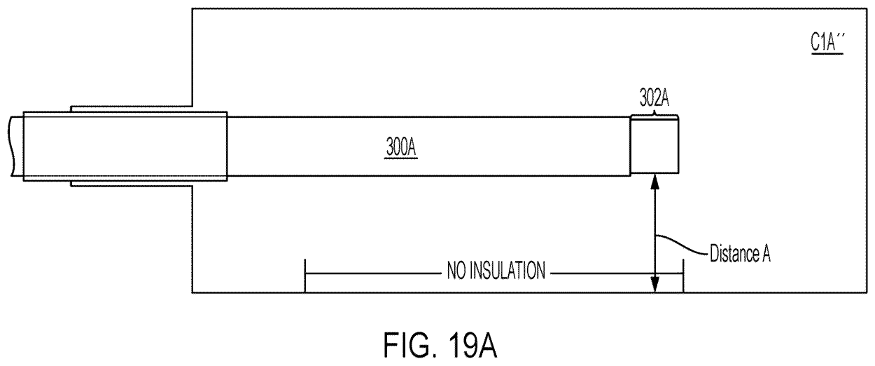

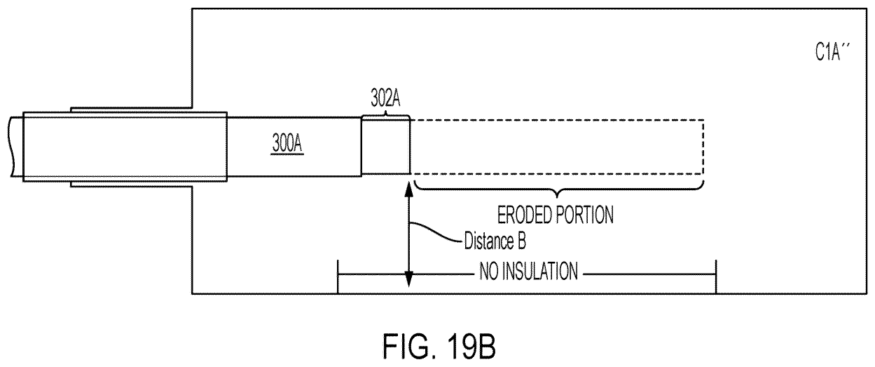

FIGS. 19A and 19B are alternative embodiments, similar in function with FIGS. 18A-18B except that the raised flattened region electrode is replaced with an inner surface of the cutout C1A″ of the electrode support member body B that is stripped of insulation for a length or distance, wherein the region of exposed conductive material functions as an electrode. This arrangement further ensures that, as the lateral surface of the exposed wire region 302 is involved in electrical arcs with the electrode comprising exposed conductive material, the spark gap distance remains substantially the same across the executed series of voltage pulses and electrical arcs. Thus, distances A and Bare substantially equal, and each spark gap therebetween is also substantially the same length as distances A and B.

In some embodiments, the spark gap shaping and related distance as the erosion of the wire conductor's exposed wire lateral surface erodes may be tuned to the magnitude of the voltage pulses generated by the voltage pulse generator. In such embodiments, an initial series of voltage pulses at a predetermined magnitude, tuned to ensure that electrical arcs are produced between the spaced-apart electrodes at the known spark gap distance. In some embodiments, the spark gap distance may change at a known rate and within one or more of a plurality of series of voltage pulses and associated arcing as the electrical arcing process is executed. Thus, as the erosion process progresses, the lateral surface of the wire conductor's exposed wire is engaged in arcing and the exposed wire region, e.g., 302A, begins to translate and traverse over the electrode (exposed metal) surface of the electrode support member. During this shortening traversal, the spark gap distances may be substantially the same for a period of pulses/arcs and/or may change across the executed pulses/arcs. Changing spark gap distances may be correlated with the known spark gap distances relating to the relative positions of the lateral surface of the exposed wire of the wire conductor and the location range of produced electrical arc engagement along the exposed conductive material region of the electrode support member that comprises an electrode.

Thus, the controller may correlate the required, or desired, voltage pulse magnitude with the known spark gap distances, or distances ranges, for an initial series of voltage pulses and associated electrical arcs and further through to the maximum allowed number of voltage pulses and/or electrical arcs for a specific device. The known spark gap distances over time and produced arcs, allows the controller to modify the voltage magnitude as the pulse numbers progress (and the spark gap distances change) in order to ensure (1) that an electrical arc occurs; and/or (2) that the pressure output resulting from the controller-initiated voltage pulse at a predetermined magnitude is within a relatively tight and controlled window. In some embodiments, the controller may determine whether sufficient electrical energy was released by an energy storage element to produce an electrical arc.

The pairs of spaced-apart spark gaps may be arranged, and wired, in a variety of ways.

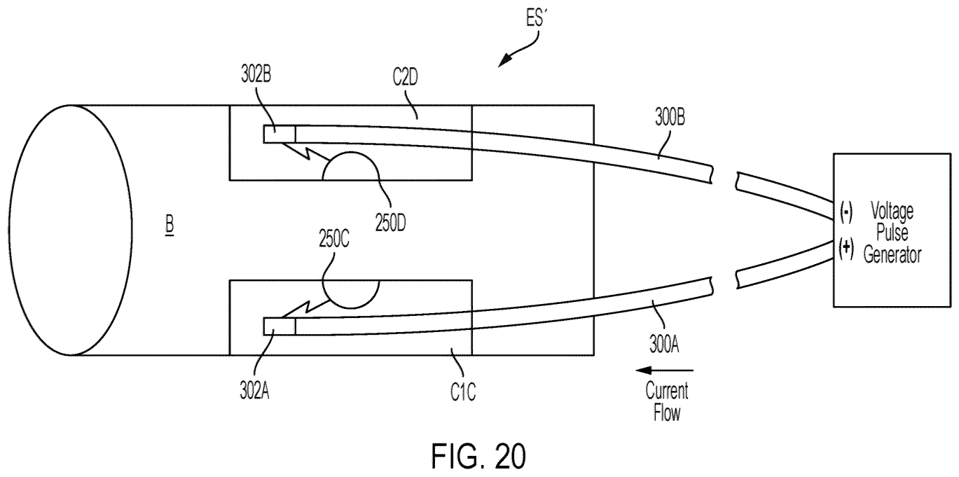

Perhaps the simplest arrangement involves the electrode support member ES' discussed above in connection with FIGS. 16A-16D. As shown in FIG. 20, the electrode support member ES' comprises a first cutout C1C with a first wire conductor 300A received within slot or channel 266 and with a first exposed wire 302A having a lateral face serving as a first electrode and positioned in a spaced-apart location relative to an exemplary exposed metal arcuate region 250C serving as a second electrode in a first spaced-apart electrode pair.

As discussed in connection with FIG. 17D, it is preferable that the spaced-apart electrodes in all embodiments described herein be located at positions that are substantially the same relative to the outer surface of the elongate member 220. Stated differently, the spaced-apart electrodes may each be located a distance from the outer surface of the elongate member 220, wherein the distance from a first spaced apart electrode in the electrode pair to the outer surface of the elongate member 220 is substantially equal to the distance from a second spaced-apart electrode of the electrode pair to the outer surface of the elongate member 220. Moreover, at least part of the lateral surface of the exposed wire section of the first wire conductor may be located between the first and second longitudinal sides of the cutout, and aligned with lateral surfaces of the first and second longitudinal sides of the cutout and with the exposed metal region. Alternatively, the two spaced-apart electrodes of an electrode pair may each be located along the outer surface of the elongate member 220.

The second cutout C2D also comprises an exposed metal arcuate region 250D which serves as a third electrode. Second cutout C2D also receives a second wire conductor 300B within slot or channel 268 and wherein a distal end of the second wire conductor 300B comprises an exposed wire region 302B with a lateral face thereof serving as a fourth electrode.

In operation, when the voltage generator initiates a voltage pulse of sufficient magnitude, current will flow through the first wire conductor 300A to the first electrode 302A and across a first spark gap from the first electrode 302A to the second electrode 250C within the first cutout C1C, creating an electrical arc and resultant pressure wave. Current will continue to flow through the conductive body B of the electrode support member ES' until reaching the third electrode at exemplary arcuate region 250D of the second cutout C2D. Current will flow across a second spark gap from the third electrode to a fourth electrode which comprises the second wire conductor 300B and its distal region of exposed wire 302B, with the current then flowing along second wire conductor 300B back to a negative terminal of the voltage pulse generator. As the current flows across the second spark gap from the arcuate region 250D to the distal region of exposed wire 302B in the second cutout C2D, an electrical arc is generated with resultant pressure wave. The second wire conductor is in operative electrical communication with a negative or ground terminal of the voltage generator and the first wire conductor is in operative electrical communication with a positive or high side terminal of the voltage generator.

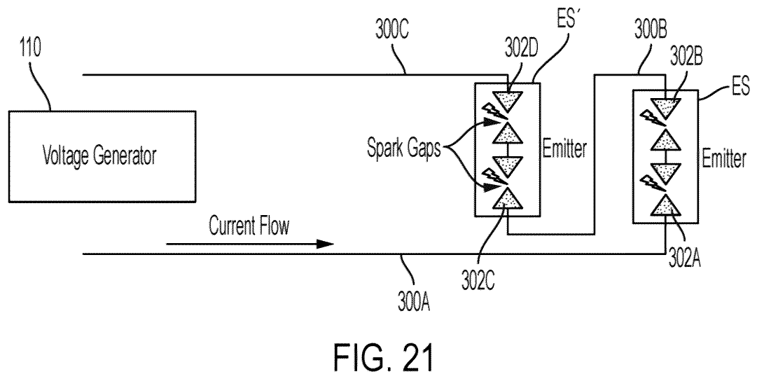

FIG. 21 illustrates an IVL system capable of performing at least one method for applying voltage pulses to the system, resulting in current flow through the exemplary system to each set of spaced-apart electrodes or emitter 302, wherein the emitters 18 are connected in a series connection. FIG. 21 illustrates two sets of spaced-apart electrodes or emitters 302A-302D defined within each support body ES or ES′. Application of a voltage pulse of sufficient magnitude and/or duration from the electric pulse generation system 110 to the sets of serially connected spaced-apart electrodes or emitters 302A-302D will result in a successive production of electrical arcs across spark gaps in an order conforming with the connection method of the electrodes with the electric pulse generation system 110. In the illustrated case, the connection method is a series connection.

With continued reference to FIGS. 15A-15E, the more proximally positioned first electrode support member may comprise electrode support member ES which, as discussed, comprises two radially spaced-apart cutouts CIA, C1B, each cutout C1A, C1B defining a pair of spaced-apart electrodes with a spark gap therebetween as described herein. The second (more distal) electrode support member may comprise ES' as described above and, like ES, comprises two radially spaced-apart cutouts C2C, C2D, each cutout C2C, C2D defining a pair of spaced-apart electrodes with a spark gap therebetween.

With continued reference to FIGS. 15A-16D, the current flow in FIG. 21 initiates at the voltage generator producing a voltage pulse of sufficient magnitude. The resulting current flows distally along a first wire conductor 300A to reach a first electrode comprising the first wire conductor's distal region of exposed wire 302A. The lateral face of the exposed wire region 302A is in spaced-apart relation with a first cutout's C1A arcuate region 250A which serves as a second electrode in the spaced-apart electrode pair as described above. As described above in connection with FIG. 17D, it is preferable that the spaced-apart electrodes in all embodiments described herein be located at positions that are substantially the same relative to the outer surface of the elongate member 220. Stated differently, the spaced-apart electrodes may each be located a distance from the outer surface of the elongate member 220, wherein the distance from a first spaced apart electrode in the electrode pair to the outer surface of the elongate member 220 is substantially equal to the distance from a second spaced-apart electrode of the electrode pair to the outer surface of the elongate member 220. Moreover, at least part of the lateral surface of the exposed wire section of the first wire conductor may located between the first and second longitudinal sides of the cutout and aligned with the exposed metal region. Alternatively, the two spaced-apart electrodes of an electrode pair may each be located along the outer surface of the elongate member 220.

The current flowing across this first spark gap will create an electrical arc and associated pressure wave.

Continuing with reference to FIGS. 15A-16D and FIG. 21, current will continue to flow through the conductive material of the body B of the more proximally located electrical support member ES until reaching a third electrode comprising a second arcuate region 250B within a second cutout C2B that is radially spaced from the first cutout C2A. A second wire conductor 300B or bridge wire comprising tantalum provides a proximal and a distal end regions wherein both end regions comprise exposed tantalum wire, with the remaining tantalum wire covered in insulation. The proximal end of the exposed tantalum wire comprises a lateral face at 302B functions as the fourth electrode in this system and which is preferably in spaced-apart relation with the third electrode defined by the second arcuate region 250B.

As the current flows from the third electrode to the fourth electrode within the second cutout C2B, a second electrical arc is formed across the spark gap and a pressure wave results.

Next, the current flows along the second wire conductor 300B comprising the tantalum bridge wire to a more distally spaced and located electrode support member ES′. The distal exposed wire 302C of the tantalum bridge wire comprises a lateral face that serves as a fifth electrode in this system and which is located within a first cutout C1C of the electrode support member ES′. The lateral face of the fifth electrode is spaced apart from a sixth electrode comprising an arcuate region 250C of exposed metal of the first cutout C1C of the electrode support member ES′. As current flows from the fifth electrode across the defined spark gap to the sixth electrode, an electrical arc is generated and a pressure wave is produced.

Current continues to flow from the sixth electrode through the conductive body B of the electrode support member ES' until reaching a seventh electrode comprising an arcuate region 250D of exposed metal within a second cutout C2D of the electrode support member ES′. An eighth electrode comprising a third wire conductor 300C having a distal end region of exposed wire 302D, wherein a lateral face of the exposed wire region 302D comprises the eighth electrode. The proximal end of the third wire conductor 300C is in operative electrical connection with a ground or low or negative terminal of the voltage generator. As current flows from the seventh electrode to the eighth electrode within the second cutout C2D of the electrode support member ES′, an electrical arc is generated across the spark gap and a pressure wave is produced. A portion of the third wire conductor 300C maybe received within slot or channel 260 of ES with a proximal end of the third wire conductor 300C placed in operative electrical connection with the voltage generator as discussed above.

Similar configurations may be produced with three or more electrode support members connected in series. For example, a proximal electrode support member ES may be serially connected via a tantalum bridge wire to a second more distal electrode support member ES which may, in turn, be connected via a second tantalum bridge wire to a distal most electrode support member ES′. The current will flow through the first proximal electrode support member ES as described above, then through the second more distal electrode support member ES in the same way, then through the distal electrode support member ES' as described above.

It is also possible to combine two (or more) pairs of electrode support members connected in series, each pair of electrode support members functioning as described above, through use of a controller and/or multiplexer that selectively applies voltage to one pair of electrode support members, then selectively applying voltage to a second pair of electrode support members.

Moreover, in each of the wiring configurations for the embodiments of electrode support members discussed above, the current flow may be reversed within a given circuit by changing the polarity for a series of voltage pulses. In other embodiments, the polarity may be reversed for each voltage pulse. The result of such a polarity change is to change which side of the spaced-apart electrode pair functions as an anode and which side functions as a cathode. Functionally, this is may provide an advantage in terms of extending the number of resultant electrical arcs may be generated between the two spaced-apart electrodes, particularly if one of the spaced-apart electrodes wears or erodes more quickly than the other electrode in the spaced-apart pair of electrodes.

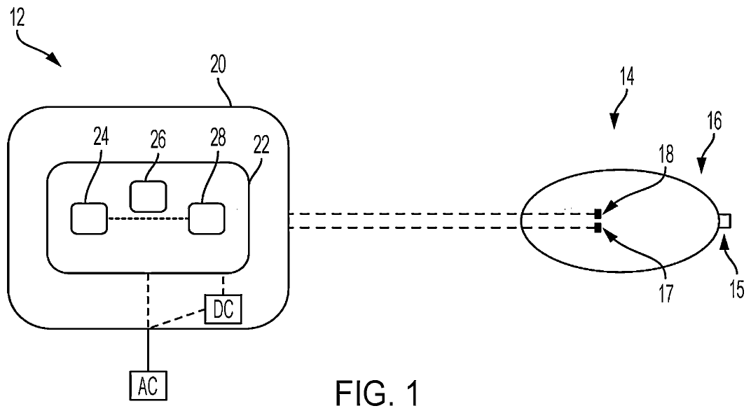

FIG. 22 illustrates a handle that may be in operative communication and connection with a console that may comprise a processor and an operatively connected memory such as described in connection with FIG. 1 for executing instructions and the voltage pulse generator 110 described above. In this embodiment, the handle comprises, or is in communication with, an EPROM (erasable read-only programmed memory) which may be in operative communication with a processor and/or a memory. The EPROM may provide therapy parameters, which may be specific for a particular model of IVL device, e.g., a balloon of specified length and/or with a specified number of electrode support members and/or spark gaps. In addition, the processor and/or memory may store therapy parameters for comparison of the progressing therapy monitored by the processor, against the stored therapy parameters. The processor may also generate connection logs, monitor number (and maximum number of allowed) pulses and may generate connection logs. FIG. 23 provides a general flow for data involving an EPROM with an IVL system of the present disclosure.

Having described certain key features of the exemplary IVL systems herein, we now turn to the functional results of those exemplary systems. A KNOWN IVL system operated in conformance with its instructions for use, and a TEST system conforming with the present disclosure were subjected to a comparative pressure output test.

The testing method and materials included comparison of TEST and KNOWN IVL devices, each including a catheter, a balloon, two radially spaced-apart pairs of electrodes within the balloon and a voltage pulse generator connected with the pairs of electrodes. The TEST balloon size included 2.5×12 mm and 4.0×20 mm. The KNOWN balloon sizes included 2.5×12 mm, 4.0×20 mm, 2.5×40 mm and 4.0×40 mm. Each tested IVL system comprised substantially similar spacing between longitudinally spaced and adjacent electrode pairs. The testing device included ONDA HNR-0500 (S/Ns 2149, Cal date 3 May 2023 and 2160, Cal date 5 May 2023) needle hydrophones without amplification. This hydrophone has an active diameter of 2.5 mm. The hydrophone was calibrated and traceable to Onda Corporation. The frequency response is flat from 0.5 MHz to 10 MHz within +/−6 dB with measurement uncertainty of 1.5 dB for frequencies 0.5-1 MHz and 1 dB for frequencies 1-10 MHz. The TEST and KNOWN devices were immersed in a water bath. A total of 1,440 voltage pulses were executed by the KNOWN system and the resulting pressure output measured, and a total of 13,320 voltage pulses were executed and the resulting pressure output measured for the TEST system using the testing method and materials.

The pressure output test method consisted of the following steps for each tested device:
1. Place the subject IVL device balloon in a water bath.
2. Position the hydrophone about 2.89 mm distal of the subject spark gap with an approximate 5 mm offset between the hydrophone and the catheter's longitudinal axis.
3. Generate a voltage pulse at a predetermined magnitude.
4. Move the catheter such that the hydrophone is positioned about 0.9375 mm proximally relative to the subject pair of electrodes' spark gap.
5. Repeat steps 1-4 until the hydrophone is positioned more than 2.89 mm proximal of the subject spark gap.
6. Repeat steps 1-5 at axial rotations of the catheter of 45, 90, 135 and 180 degrees.

### Comparative Testing and Select Features

### Pressure Output Variability

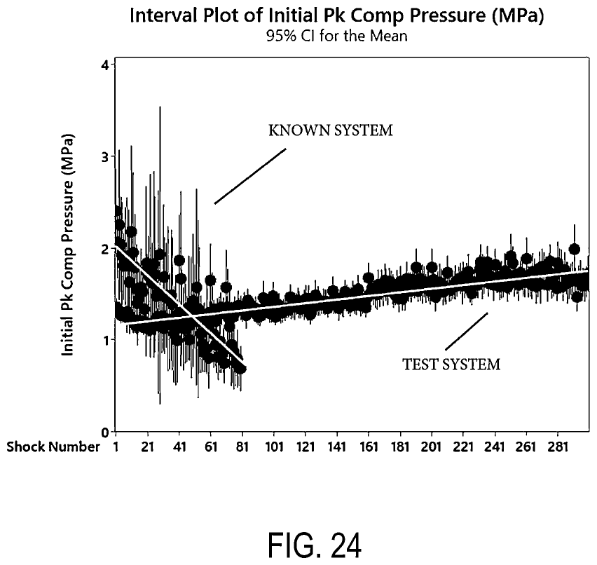

FIG. 24 provides a data plot of pressure output (initial peak pressures) for a KNOWN IVL system and a TEST IVL system that conforms with the present disclosure. As is immediately visually apparent, the KNOWN system's pressure output varies greatly. In contrast, the TEST system pressure output data is relatively tightly controlled, with relatively low variation compared with the KNOWN system's variation. A data summary is provided in TABLE 1:

TABLE 1

| | Number | Mean | Standard | Coefficient of Variation |
|---|---|---|---|---|
| System | of Tests | (MPa) | Deviation (SD) | (CV): SD I Mean |
| KNOWN | 1,440 | 1.3945 | 0.5475 | 39.3% |
| TEST | 13,320 | 1.4468 | 0.3441 | 23.8% |

Thus, the coefficient of variation (“CV”), which is an indicator of variability within data sets is 39.3% for the KNOWN system's pressure output data and is 23.8% for the TEST system's pressure output data. Accordingly, it can be reasonably concluded that the TEST system's pressure output data is more controlled and substantially less variable than the KNOWN system's pressure output data. Using the key features in an IVL system will, therefore allow for a CV in a series of pressure output data that is less than 35%. More preferably, the CV in a series of pressure output data will be less than 30% and still more preferably, the CV in a series of pressure output data will be less than 25%. As noted supra, the IVL systems described herein provide for more durable (far more pulses/electrical arcs/pressure waves per catheter), more efficient (higher frequency of pulses/electrical arcs/pressure waves), and much more controlled pressure output from the produced pressure waves than the known IVL systems.

### Force Required to Traverse Vasculature

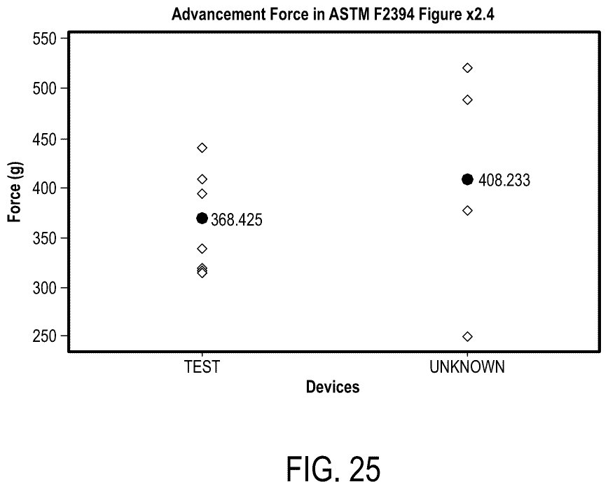

The TEST system and KNOWN system (Shockwave Medical C2 IVL catheter) were subjected to force standard tracking testing using the ASTM F2394 Tracking Fixture as shown in FIG. 25. The tested systems each comprises a total number of two emitters comprising spaced-apart electrodes. The tested systems were each translated through the illustrated standard fixture while tracking the force required to move the tested catheters from the beginning to the end of the fixture. The TEST and KNOWN systems were both two-emitter designs (two spaced-apart electrode pairs, wherein the spaced-apart electrode pairs are longitudinally spaced apart from each other), and the balloon sizes were both 3.0 mm×20 mm for both of the TEST and KNOWN systems. The ASTM F2394 Tracking Fixture was filled with water to lubricate. The TEST and KNOWN catheters were tracked over a wire through the model until the distal tip reached the end of the model. The peak force was the value that was recorded and observed in the plot as illustrated in FIG. 25. This comparative testing measures relative resistance through tortuous vasculature.

As shown, the mean peak force for the TEST system was 368.425 grams and for the KNOWN system, the mean peak force was 408.233 grams. This represents a 9.75% decrease in force required for the TEST system compared with the KNOWN system. The % difference between the two mean peak force values is 10.8%.

Accordingly, the mean peak force for embodiments of the TEST system through the ASTM F2394 Tracking Fixture is approximately 9.75% less than the mean peak force for the KNOWN system.

Certain embodiments of the TEST system may comprise a mean peak force through the ASTM F2394 Tracking Fixture that is at least 9.5% less than the mean peak force for the KNOWN system.

Certain embodiments of the TEST system may comprise a mean peak force through the ASTM F2394 Tracking Fixture that is at least 9% less than the mean peak force for the KNOWN system.

Certain embodiments of the TEST system may comprise a mean peak force through the ASTM F2394 Tracking Fixture that is at least 8% less than the mean peak force for the KNOWN system.

Certain embodiments of the TEST system may comprise a mean peak force through the ASTM F2394 Tracking Fixture that is at least 6% less than the mean peak force for the KNOWN system.

Certain embodiments of the TEST system may comprise a mean peak force through the ASTM F2394 Tracking Fixture that is at least 5% less than the mean peak force for the KNOWN system.

Accordingly, the TEST system traversed the test fixture from a beginning to an ending of the fixture with a peak force less than 405 grams.

Further, the TEST system traversed the test fixture from a beginning to an ending of the fixture with a peak force less than 400 grams.

The TEST system also traversed the test fixture from a beginning to an ending of the fixture with a peak force less than 375 grams.

The TEST system also traversed the test fixture from a beginning to an ending of the fixture with a peak force less than 370 grams.

As a result, the peak advancement force for the TEST system may be within a range of about 400 grams to about 405 grams.

The peak advancement force for the TEST system may also be within a range of about 375 grams to about 405 grams.

The measured peak advancement force is for the TEST system may also be within a range of about 370 grams to about 405 grams.

In addition, TABLE 2 below provides a partial summary listing of functional improvements and enhancements provided by embodiments of the present disclosure relative to a KNOWN system, with a partial listing of the disclosed features leading to the improvements and enhancements.

TABLE 2

| | | | Presently Disclosed |
|---|---|---|---|
| | | Presently Disclosed | Features Enabling the |
| Functional Result | KNOWN System | Embodiments | Functional |
| Crossing Profile | Outer diameter of | Outer diameter | 1. Electrodes not |
| | 0.044″-0.047″ | (“OD”) about | fully |
| | | 0.044″ | circumferential; |
| | | or less for 2.5 mm | wires m same |
| | | diameter balloon. | circumferential |
| | | OD about 0.045′ or | plane as the body of |
| | | less for 3.0 mm and | the electrode |
| | | 3.5 mm | support member. |
| | | diameter | 2. Tapering member |
| | | balloons. | in the proximal |
| | | | inflation region of |
| | | | the balloon. |
| Deliverability | | | 1. Smaller crossing |
| | | | profile in the |
| | | | folded balloon |
| | | | region; less push |
| | | | force required. |
| | | | 2. Smaller distal |
| | | | outer diameter at |
| | | | distal tip than at the |
| | | | proximal end of the |
| | | | sealed balloon. |
| | | | 3. Increased |
| | | | flexibility in at least |
| | | | the balloon region |
| | | | compared with |
| | | | KNOWN - 1 less |
| | | | layer of material than |
| | | | KNOWN. |
| | | | 4. Improved axial |
| | | | push force without |
| | | | kinking due to |
| | | | presence of |
| | | | tapering outer |
| | | | member and |
| | | | stiffening layer in |
| | | | region of the |
| | | | balloon. |
| Efficiency/ | 1 pulse/second | 1-5 pulses/second | 1. Control of |
| Frequency of Pulses | | with 2 pulses/second | spark gap lengths. |
| | | preferred | 2. Minimal spark |
| | | | gap shorting. |
| | | | 3. Tantalum |
| | | | bridge wire. |
| | | | 4. Insulation |
| | | | covering |
| | | | electrode support |
| | | | bodies, resulting |
| | | | in less gas |
| | | | produced during |
| | | | arcing. |
| Variability of | Greater than 39% CV | Less than 24% CV | 1. Control of |
| Pressure Output | | | spark gap |
| | | | lengths. |
| | | | 2. Minimal spark |
| | | | gap shorting. |
| | | | 3. Tantalum |
| | | | bridge wire. |
| | | | 4. Changing |
| | | | voltage to |
| | | | accommodate |
| | | | pressure over |
| | | | number of pulses. |
| Durability (maximum | Maximum 120 pulses | Up to 500 pulses per | 1. Control of |
| number of pulses per | per catheter for | catheter for coronary | spark gap |
| catheter). | coronary devices. | and peripheral devices. | lengths. |
| | Maximum 300 pulses | | 2. Minimal spark |
| | per catheter for | | gap shorting. |
| | peripheral devices. | | 3. Tantalum bridge |
| | | | Wire. |
| | | | 4. Insulation |
| | | | covering electrode |
| | | | support bodies, |
| | | | resulting in less gas |
| | | | produced during |
| | | | arcing. |
| | | | 5. Less variable |
| | | | pressure output than |
| | | | known systems. |

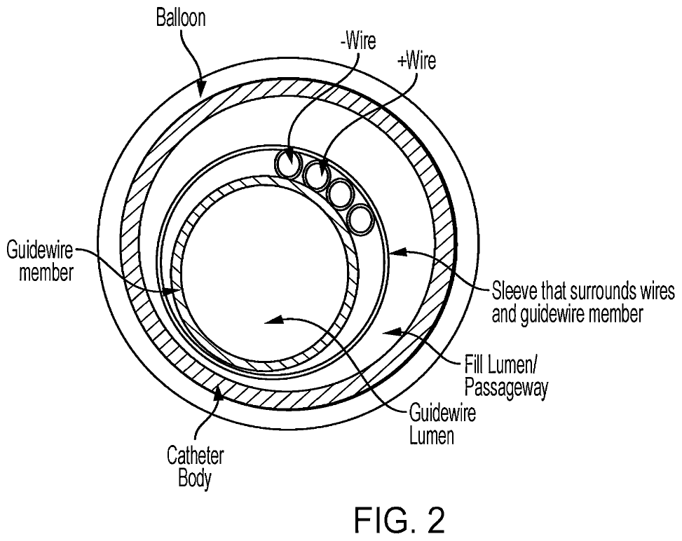

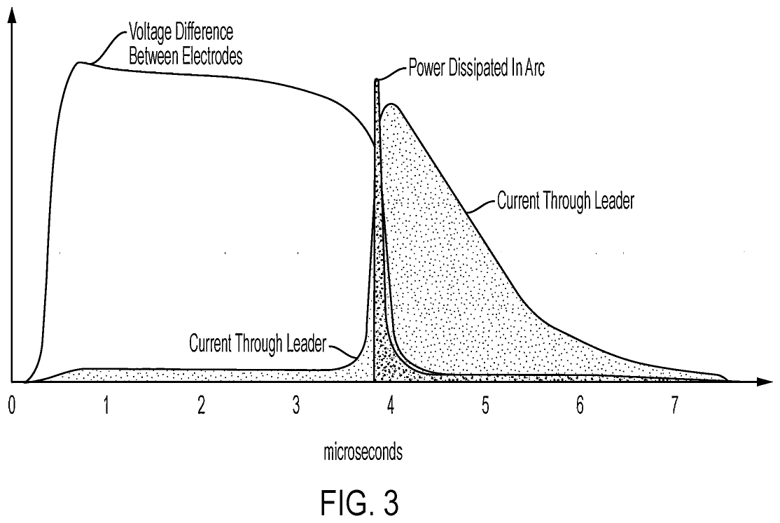

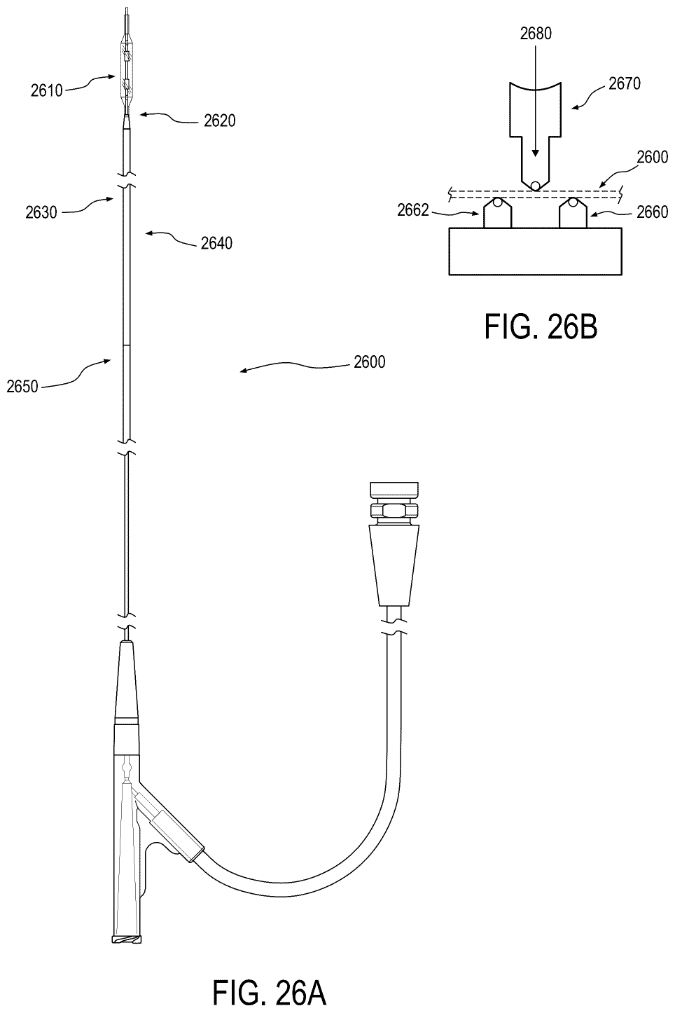

Various tests have been performed on IVL catheters to examine structural characteristics of the IVL catheters related to pushability and/or deliverability through coronary vessels. For example, illustrated in FIG. 26A-26B are testing positions 2610-2650 of several working embodiments of an IVL catheter 2600 that were tested on a testing apparatus 2660 according to aspects of the present disclosure. The catheter 2600 can be any of the catheters described herein and described in relation to FIGS. 1-25. The portion of the IVL catheter 2600, which is to be inserted into or navigate coronary vessels, is an elongated portion (e.g., the elongate member 220 of FIG. 6) of the IVL catheter 2600. In particular, the hypotube (e.g., the hypotube 402 of FIGS. 12B1-12B3) and/or a polymer tube (e.g., the polymer tube 430 of FIG. 12B3, which can be formed of the outer wall polymer 420′ extending along the polyimide PC toward the balloon 200 from the bonded section 403) that may be inserted into the coronary vessels. The hypotube 402 is generally made of stainless steel, meaning that the hypotube 402 is relatively stiffer than the polymer tube 430 and provides strong columnar strength and pushability characteristics to the IVL catheter 2600. Specifically, the main part of the IVL catheter 2600, which navigates through coronary vessels, is the polymer tube 430 and possibly a portion of the hypotube 402. As mentioned above, while the hypotube 402 can be formed of stainless steel, it is understood that other metals, alloys, polymers, ceramics, composites, combinations or modifications therefore, whether coated or uncoated with a polymer, ceramic or other coating (e.g., to improve lubricity, hydrophobicity or hydrophilicity, or a combination thereof, relative to an uncoated base material), can form the elongate structure of the hypotube 402.

As illustrated, five positions 2610-2650 are selected. Specifically, the first position 2610 is positioned at a balloon (e.g., the balloon 200 of FIGS. 6, 7A, and 7B), which is coupled with, e.g., fixedly bonded to, a distal portion of the polymer tube 430 of the IVL catheter 2600. The first position 2610 may be positioned in a region extending from the distal tip of the IVL catheter 2600 to about 1 cm proximal of the balloon. Distal to or near the bonded portion, the polymer tube 430 may be tapering out and is not sealed or bonded against any portion of an elongate member (e.g., the elongate member 220 of FIGS. 6, 7B, 9-11, 12D, 14A, 14B, and 17A). The elongate member may be received within and extended distally from the distal tip of the tapering portion of the polymer tube 430. In particular, the distal portion and the proximal portion of the balloon are glued or bonded to the corresponding portions of the distal portions of the polymer tube 430 and the distal portion of the elongate member, respectively.

The balloon length may be 12 mm, 20 mm, or any suitable length to perform a lithotripsy operation. For instance, the balloon length can be equal to or greater than 10 mm, 12 mm, 14 mm, 16 mm, 18 mm, 20 mm, 22 mm, 24 mm, 26 mm, 28 mm, 30 mm, 32 mm, 34 mm, 36 mm, 38 mm, 40 mm, 42 mm, 44 mm, 46 mm, 48 mm, 50 mm, 52 mm, 54 mm, 56 mm, 58 mm, 60 mm, or is in a range between any two of the foregoing, e.g., from a selected first one of the foregoing to a selected second one of the foregoing. The presence of a tapering outer member of the balloon, which is positioned near the distal tip of the elongate member, provides stiffness. The tapering section of the tapering outer member provides a relatively smaller crossing profile in a wrapped balloon configuration, thereby providing additional pushability and strength in the region of the outer member of the balloon. Further, kinking of the wrapped balloon may be prevented while the distal tip (e.g., the distal tip 218 of FIG. 7A) of the polymer tube 430 advances in the coronary vessel.

One or more electrode support members ESs (ES or ES' of FIGS. 6, 7B, 9, 11, 12D, 14A, 14B, and 15A-15D) may surround the elongate member as illustrated in FIGS. 6, 7B, 9, 11, 12D, 14A, 14B, and 15A-15D. Further, the ESs may be stainless steel and are coated with an insulating material such as a polymer or a blend of polymers or other materials, including but not limited to an adhesive, polyimide or other high temperature resistant flowable non-conductive material. Other metals or alloys can be used for the electrode support members (ES, ES′).

The middle portion (e.g., segments 212, 214 in FIG. 7A) of the balloon (e.g., balloon 200) may be pleated and folded around the elongate member and the ESs, thereby increasing the bending stiffness in the middle portion and providing further deliverability and pushability of the balloon portion, which is positioned near the distal tip of the polymer tube 430.

Bending stiffness of each portion of the IVL catheter 2600 was assessed at each of the five positions 2610-2650 based on 3-position bending tests, in compliance with ASTM F2606-08 standard (approved and published in August 2021). For example, the term “bending stiffness” as used herein is sometimes referred to in the art as “bending flexibility” as defined in ASTM F2606-08, Section 8.4. However, applicable test standards are not limited to the ASTM F2606-08 standard, but can include other standards as appreciated by persons having skill in the IVL catheter 2600 bending testing. The testing apparatus 2660, schematically illustrated in FIG. 26B, has a first support 2662 and a second support 2664, where the catheter 2600 is placed, and an upper strain (or load) applicator 2670, which applies vertical force to the catheter at a position centered between the supports 2662, 2664 from above in the direction of an arrow 2680 and releases the vertical force in a direction opposite to the direction of arrow 2680. The span between the two supports 2662, 2664 may be one inch and a rate of application of the vertical force by the upper strain applicator may be one inch per minute. A maximum deflection may be limited to five millimeters (mm), in certain test configurations. Prior to load tests, the IVL catheter 2600 may have been soaked in 37° C. water for at least about 2 hours. It will be understood that the separation of the supports 2662, 2664 can be greater or lesser than one inch, the application of strain force rate can be faster or slower than one inch per minute, maximum deflection may be limited to five mm or can be greater or lesser than five mm, and the IVL catheter 2600 may have been soaked in water at other (e.g., higher or lower) temperatures and for shorter or longer duration than about 2 hours. More generally, the testing condition may not be limited to these described herein but can be changed and include others to mimic, e.g., in situ environments other than a human body.

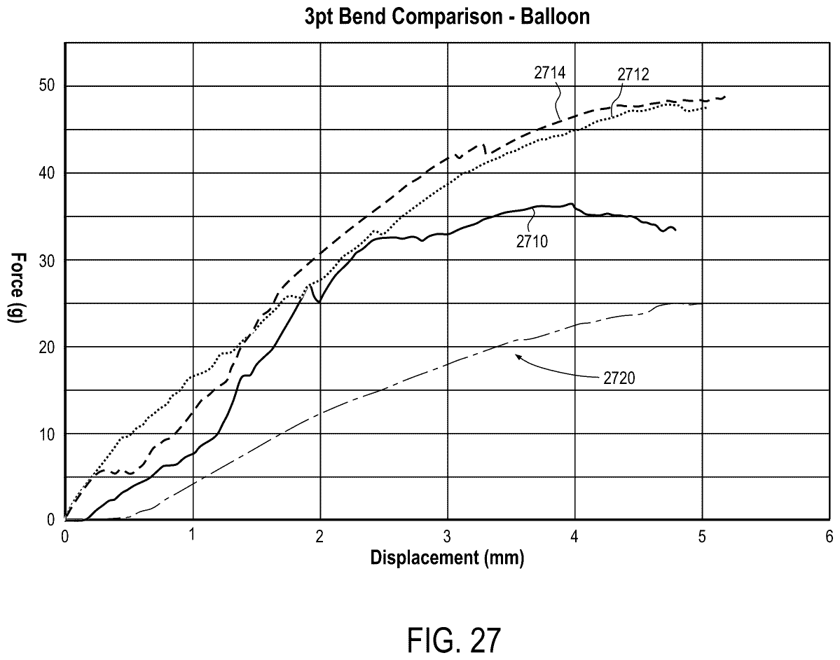

During testing, each testing position 2610-2650 of the catheter 2600 is centered between the two supports 2662, 2664 and the upper strain applicator 2670 applies vertical strain force to a mid-point of the specimen (or segment thereof) under test following the strain force rate (e.g., 1 in/min). The amount of vertical force applied to the specimen under test by the applicator 2670 was measured as the catheter 2600 deformed. Several tests were performed at each testing position 2610-2650 on several specimens (e.g., several working embodiments of IVL catheter 2600) and test results (e.g., vertical displacement of the simply supported segment of the specimen under test as a result of the strain force, or vertical load, applied in a unit of grams (g) to the segment under test) were obtained for each specimen. The measured force-displacement curves (e.g., observed for each specimen at a respective segment) were combined, e.g., averaged, to provide force-displacement curves 2710-3120 shown in FIGS. 27-31. The bending stiffness of a segment under test can be estimated by dividing a total force applied by a vertical displacement of the segment under test. Thus, the unit of the bending stiffness can be force-per-unit-displacement, e.g., grams-force per millimeter (gf/mm), as used herein. Throughout this description, where units of g/mm is used in connection with describing a bending stiffness (or bending flexibility), it will be understood to reflect a unit of grams-force per millimeter, consistent with a unit-force-per-unit-displacement. ASTM F2606-08, Section 8.4, accounts for a modest non-linearity in the substantially linear region of the force-displacement curves 2710-3120 shown in FIGS. 27-31 by imposing an upper threshold value of R², e.g., R²≥0.9.

Individual test results at the first position 2610 with 12 mm balloons (e.g., balloon 200) is illustrated as force-displacement curves 2710, 2712, and 2714 in FIG. 27, and averages of the test results at the first position 2610 with 20 mm balloons (e.g., balloon 200) are illustrated as the force-displacement curve 2720 in FIG. 27. The horizontal axis represents displacements in units of millimeters (mm), and the vertical axis represents vertical force in unit of grams (g). With respect to different sizes of the balloons, the center of the balloons may be selected as the first position 2610. In other words, the first position 2610 of the 12 mm balloon may be different from the first position 2610 of the 20 mm balloon with respect to the distal tip (e.g., the distal tip 218) of the IVL catheter 2600.

In the region between 0 and 1 mm, the force-displacement curve 2720 has a relatively shallow slope (about 3 g/mm and 4 g/mm) than the slope (about 6 g/mm) in the region between 1 to 5 mm according to the force-displacement curve 2720. These slopes are calculated for wider ranges. In consideration of smaller ranges, instantaneous slopes at different points may be calculated to be steeper than or shallower than the average slopes in the whole range. Further, a confidence interval may be considered to have a range of slopes with different confidence levels for the whole range.

For example, an effective slope across the whole displacement range may be greater than 5 g/mm based on the force-displacement curve 2720 showing results for IVL catheters with the 20 mm balloons (e.g., balloon 200). On the other hand, the average slope in the whole range may be about 8.7 g/mm for IVL catheters with the 12 mm balloons (e.g., balloon 200). The outer portion of the balloons can be surmised to have more bending stiffness than the middle portion of the balloons, as the end portions of the 12 mm balloons are closer to the upper load applicator 2670 than the end portions of the 20 mm balloons. However, individual test results of the force-displacement curves 2710, 2712, and 2714 (e.g., for the IVL catheters with 12 mm balloons) show more fluctuations than the force-displacement curve 2720 (e.g., for the IVL catheters with 20 mm balloons), as expected since curve 2720 reflects an average curve over a plurality of measurements. Nevertheless, based on the comparison, the bending stiffness at the first position 2610 of IVL catheters having the 12 mm balloon is generally observed to be greater than the bending stiffness at the first position 2610 of IVL catheters having the 20 mm balloon.

Further, at the center of the balloon (e.g., the balloon 200), the balloon is pleated and folded. Thus, the outer diameter of the center portion of the balloon can be greater than the outer diameter of the corresponding portion of the elongate member 220. Thus, it can be surmised that the bending stiffness at the first position 2610 may be higher than the bending stiffness at the two end portions of the balloon without the pleated and folded portions.

A range of bending slopes may be obtained according to various statistical analyses. For example, with the 95% confidence interval, the average slope may be greater than or equal to

$$μ - 1.96 \frac{σ}{\sqrt{n}}$$

and less than or equal to

$$μ + 1.96 \frac{σ}{\sqrt{n}} ,$$

where μ is the mean slope observed among a plurality of sample measurements at a given location, σ is the standard deviation observed among the plurality of sample measurements, and n is the number of sample measurements in the plurality of sample measurements (sometimes referred to in the art as a sample size). Likewise, with the 99% confidence interval, the average slope may be greater than or equal to

$$μ - 2.576 \frac{σ}{\sqrt{n}}$$

and less than or equal to

$$μ + 2.576 \frac{σ}{\sqrt{n}} .$$

Based on the test results of IVL catheters having a 12 mm balloon, the average bending stiffness was observed to be about 8.7 g/mm, and the standard deviation was observed to be about 0.83 g/mm with a sample size of 3. Thus, based on the statistical analysis of these three sample measurements, the range of the bending stiffness is from 7.76 g/mm to 9.64 g/mm with the 95% confidence interval. With the 99% confidence interval, the range of the bending stiffness is from 7.47 g/mm to 9.93 g/mm. In a case where the number of tests is increased, the confidence range may be shortened. Further, when the confidence level increased, the confidence interval may be lengthened.

Additionally or alternatively, the maximum observed bending stiffness was 9.68 g/mm and the minimum observed bending stiffness was 8.10 g/mm based on the actual test results.

It is noted that the average slope of the approximately linear region of the curves, pursuant to Section 8.4 of ASTM-F2606, shown in the drawings may be identified as a bending stiffness indicating a measure of resistance to bending of the IVL catheter 2600. The bending stiffness may depend on the geometrical structure and the materials of the IVL catheter 2600, and thus provides a suitable means of comparing “bendability” or “stiffness” of one device with another device. On the other hand, a bending modulus or a flexural modulus refers to a material property relating to how much a material resists bending, e.g., without regard to geometry. Accordingly, bending modulus provides a convenient means for comparing stiffness of one material against another material, but not necessarily for comparing stiffness or bendability of components, each of which may have a unique configuration (e.g., geometry) and combination of materials (e.g., each having its own flexural modulus). Further, it is noted that the bending stiffness generally has a unit of force per length, e.g., grams (g) per millimeter (mm), while bending modulus and flexural modulus have a unit of stress, e.g., force per area. In consideration of the comparatively longer longitudinal length than the small diameter in the cross-sectional area of the IVL catheter 2600, and in consideration of possible internal material and structural differences between IVL catheters being compared with each other, the bending stiffness is used herein rather than a bending modulus or a flexural modulus. In other respects, other measures such as bending modulus or the flexural modulus may be used to identify longitudinal stiffness, which may be used to indicate pushability and deliverability of the IVL catheter 2600 within coronary vessels.

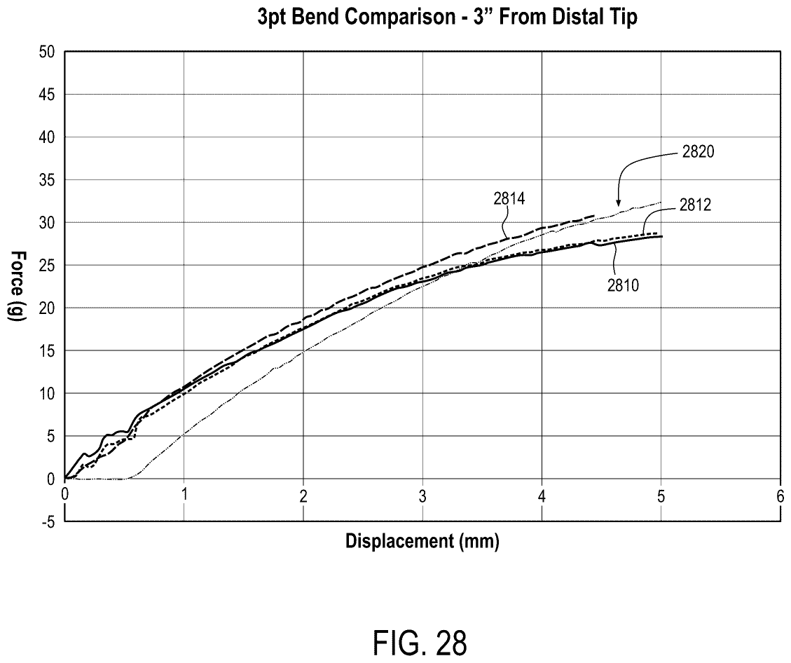

Referring now to FIG. 28, test results at the second position 2620, which is located 3 inches from the distal tip (e.g., the distal tip 218) of the IVL catheter 2600 are described. FIG. 28 illustrates force-displacement curves 2810, 2812, and 2814, which are individual test results for three samples of IVL catheters, each having 12 mm balloons (e.g., balloon 200), and force-displacement curve 2820, which is an average slope of three sample measurements of an IVL catheter having 20 mm balloons (e.g., balloon 200). Since the proximal bonding portion (e.g., proximal portion 204) of the balloons is positioned well less than 3 inches from the distal tip, the second position 2620 is proximally located relative to the balloon. Generally speaking, the second position 2620 may be positioned in a region extending proximally from the proximal portion of the balloon to about 2 cm proximal of the rapid exchange port. The wall thickness of the catheter in this region may be substantially constant therethrough. Accordingly, the bending stiffness can be considered as being substantially constant longitudinally along this region. The bending stiffness for this region is reflected in the test results discussed further below and shown in FIG. 28. Testing procedures similar to those described above in connection with testing at the first position 2610 were used to obtain the data shown in FIG. 28.

Based on the individual test results from the force-displacement curves 2810, 2812, and 2814, the average slope or the bending stiffness is about 6 g/mm (as determined consistent with ASTM-F2606, Section 8.4) a the IVL catheter having 12 mm balloons, while the average observed slope is slightly greater than 6 g/mm with the 20 mm balloons (reflected by the curve 2820). Due to the substantially similar structure of the polymer tube (e.g., the polymer tube 430) of the IVL catheter 2600 at the second position 2620 regardless of the balloon size, the overall average slopes of catheters having 12 mm balloons and catheters having 20 mm balloons are similar to each other. The segment of the polymer tube between the proximal balloon shoulder and rapid exchange port (RX Port), e.g., from approximately 0.5″ proximal of the balloon to 0.5″ distal of the Rx Port is a region of substantially constant bending stiffness.

The average bending stiffness may be measured based on a corrected curve, e.g., according to ASTM-F2606-08, which removes or compensates for the initial loading of the test specimen between the two supports 2662 and 2664. Further, the average bending stiffness may be measured at any substantially linear portions of the force-deflection curve (e.g., where R²≥0.9), which can be where displacement is within a range from 0 mm to 1 mm, a range from 1 mm to 2 mm, a range from 2 mm to 3 mm, a range from 3 mm to 4 mm, a range from 4 mm to 5 mm, or any combination of one or more of the foregoing ranges, which are considered to be linear according to the standard described in ASTM F2606-08.

Based on testing of IVL catheters with a 12 mm balloon, the average bending stiffness is observed to be about 9.6 g/mm, and the standard deviation is observed to be about 0.2 g/mm with the sample size of n=3. Thus, according to the statistical analysis, the range of the bending stiffness is from 9.37 g/mm to 9.82 g/mm with the 95% confidence level. With the 99% confidence level, the range of the bending stiffness is from 9.30 g/mm to 9.90 g/mm. On the other hand, the maximum bending stiffness was 9.85 g/mm and the minimum bending stiffness was 9.50 g/mm based on the actual test results.

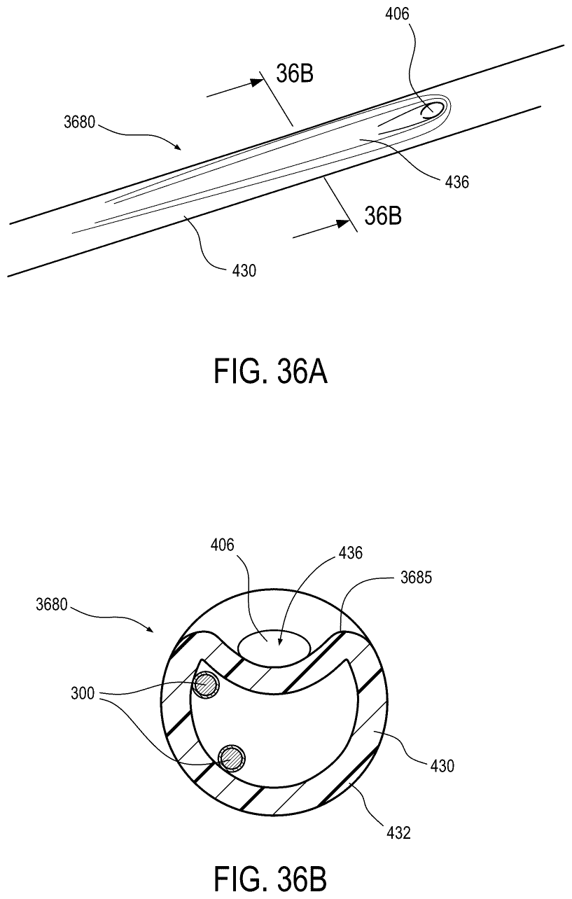

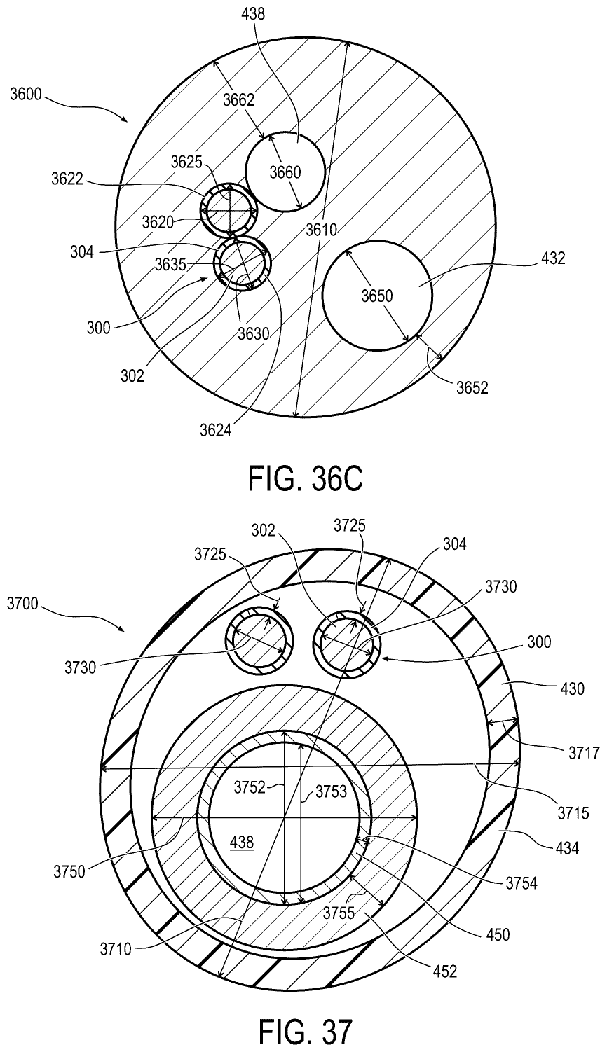

The structure (as illustrated in the cross-sectional view 3700 in FIG. 37) within the polymer tube (e.g., the polymer tube 430 FIGS. 12B2 and 12B3) at the second position 2620, which is located 3 inches from the distal tip (e.g., the distal tip 218), may have two lumens (e.g., the lumens having the outer diameters 3720 and 3730 as illustrated in FIG. 37) for wire conductors 300 and a lumen (e.g., the lumen having the outer diameter 3750 as illustrated in FIG. 37) for the guidewire (e.g., the guidewire 15 of FIG. 1) therein. The saline solution may pass through inside of the polymer tube 430. Alternatively, the wire conductors 300 are not disposed within individual lumens but are disposed within a lumen 432 of the polymer tube 430. At the second position 2620, the two lumens for (and/or the wire conductors 300 when there are no lumens for the wire conductors 300) may freely float within or be fixed to the inside surface of the outer wall (e.g., the outer wall 3717 in the cross-sectional view of FIG. 37) of the polymer tube 430, and the lumen (e.g., the guidewire lumen 438 of FIG. 36C) for the guidewire may be surrounded by the wall 434 of the polymer tube 430 (e.g., FIG. 37). The polymer tube 430 or layer surrounding the guidewire lumen 438, such as an inner polymer tube 452, is separate from the inside surface of the outer wall 434 of the polymer tube 430. In an aspect, the guidewire lumen 438 may not reach the second position 2620. These various structures may affect the bending stiffness at the second position 2620.

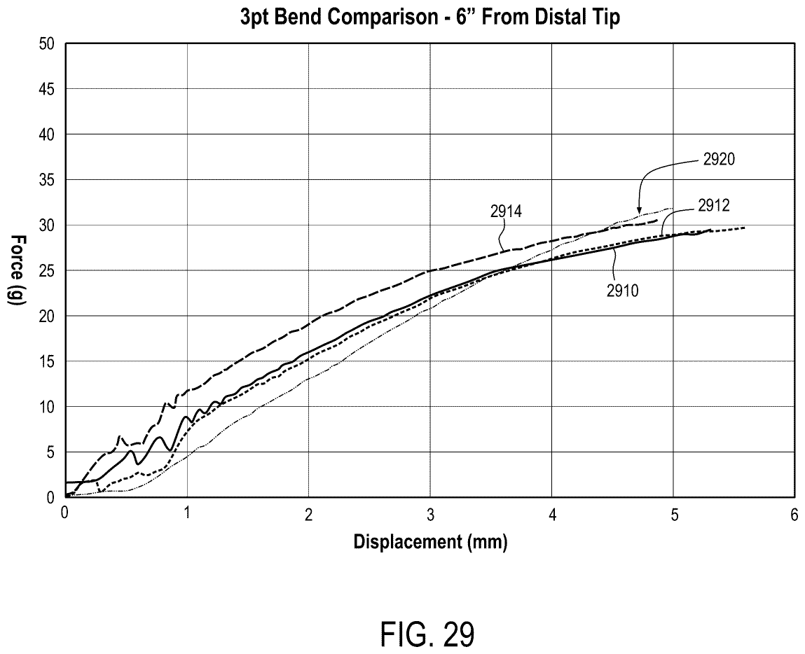

Referring now to FIG. 29, test results at the third position 2630, which is located 6 inches from the distal tip (e.g., the distal tip 218) of the IVL catheter 2600, are described. FIG. 29 illustrates force-displacement curves 2910, 2912, and 2914, which are individual test results for IVL catheters with a 12 mm balloon (e.g., balloon 200), and force-displacement curve 2920, which shows an averages of =test results for IVL catheters with a 20 mm balloon (e.g., balloon 200). The third position 2630 can be positioned distal to the rapid exchange port, through which the guidewire can enter into the polymer tube. The third position can be in a region extending from about 2 cm distal to the rapid exchange port to about 2 cm proximal of the rapid exchange port. Thus, at the third position 2630, there are four lumens inside the polymer tube 430. For example, two of them are for wire conductors 300, the third one is for the guidewire, and the fourth lumen is for saline solution. In an aspect, the inside of the polymer tube 430 may be filled or become solid except the four lumens. Stated another way, the wall 434 of the polymer tube 430 has a thickness such the wall 434 extends from one side to the other through a center of the polymer tube 430 except for the identified lumens.

Based on the individual test results from the force-displacement curves 2910, 2912, and 2914, the bending stiffness is observed to be about 6 g/mm over the whole range (e.g., 0 to 5 mm displacement) for IVL catheters having a 12 mm balloon, while the bending stiffness is slightly over 6 g/mm for IVL catheters having a 20 mm balloon. Due to the substantially similar structure of the polymer tube 430 of the IVL catheter 2600 regardless of the balloon size, the overall average slopes between the IVL catheters having 12 mm balloons and IVL catheters having 20 mm balloons are similar to each other at the third position 2630.

Compared to the average bending stiffness and the confidence intervals of the bending stiffness at the second position 2620, however, the confidence intervals of the bending stiffness at the third position 2630 are much larger and the average bending stiffness is smaller. Stated differently, the observed bending stiffness at the third position 2630 exhibited greater variability than the observed bending stiffness at the second position 2620.

Based on the test results at the center of the 12 mm balloons, the average bending stiffness is about 8.5 g/mm over a range of 0 to 5 mm displacement, and the standard deviation is about 0.9 g/mm with the sample size of 3. Thus, based on the statistical analysis, the range of the bending stiffness is from 7.48 g/mm to 9.52 g/mm with the 95% confidence level. With the 99% confidence level, the range of the bending stiffness is from 7.16 g/mm to 9.84 g/mm. On the other hand, the maximum bending stiffness was 9.20 g/mm and the minimum bending stiffness was 7.46 g/mm based on the actual test results.

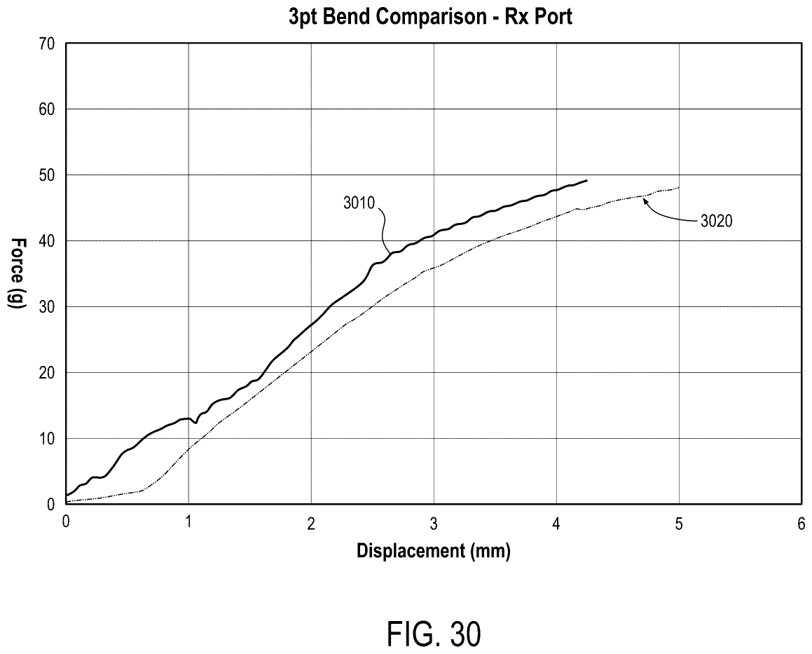

Now referring to test results at the fourth position 2640, which is located at a rapid exchange port (e.g., the RX port 406 of FIGS. 12C, 13A, and 13B) of the IVL catheter 2600, as illustrated in FIG. 30, the force-displacement curve 3010 shows average test results for IVL catheters with 12 mm balloons, and the force-displacement curve 3020 shows average test results for IVL catheters with 20 mm balloons. The fourth position 2640 is positioned at or proximal to the rapid exchange port, through which the guidewire can enter into the polymer tube 430 and/or the polyimide conduit PC. In an aspect, the fourth position 2640 may be positioned in a region extending from about 2 cm proximal of the rapid exchange port to 2 cm distal of the hypotube. The wall thickness in this region may be substantially constant therethrough, thereby the bending stiffness being substantially constant in the region in the tests below. The rapid exchange port may be about 10 inches from the distal tip (e.g., the distal tip 218) of the IVL catheter 2600. Thus, at the fourth position 2640, there can be three lumens in the polymer tube 430. For example, two of them can facilitate respective wire conductors 300, and the other lumen can convey a saline solution. In an aspect, the thickness of the outer wall (e.g., the outer wall polymer 420 or 420′ of FIGS. 12B2 and 12B3) of the polymer tube 430 may have been thickened from the proximal portion of the polymer tube 430 toward the rapid exchange port 406. Further, the inside of the polymer tube 430 is filled with the material thereof except the three lumens. Stated another way, the wall 434 of the polymer tube 430 has a thickness such that the wall 434 extends from one side to the other through a center of the polymer tube 430 except for the identified lumens. Such structure can be obtained, for example, by over-molding the identified lumens with the material of which the wall 434 of the polymer tube 430 is formed.

Based on the test results from the force-displacement curve 3010, the average bending stiffness is observed to be above 10 g/mm over the whole range for IVL catheters with the 12 mm balloons, while the average slope is slightly below 10 g/mm based on the test results for IVL catheters with 20 mm balloons from the force-displacement curve 3020.

Based on the test results for IVL catheters with the 12 mm balloons, the average bending stiffness is about 15.1 g/mm, and the standard deviation is about 1.08 g/mm with the sample size of 3. Thus, according to the statistical analysis, the range of the bending stiffness is from 13.88 g/mm to 16.32 g/mm with the 95% confidence interval. With the 99% confidence interval, the range of the bending stiffness is from 13.49 g/mm to 16.71 g/mm. On the other hand, the maximum bending stiffness was 16.39 g/mm and the minimum bending stiffness was 14.43 g/mm based on the actual test results.

With the 99.99% confidence level, the bending stiffness may be from 11.36 g/mm to 18.84 g/mm. Thus, it is almost statistically appropriate to say that the bending stiffness is greater than 6 g/mm and less than 20 g/mm.

Due to the thickened outer wall 434 of the polymer tube 430 and the solid inside area other than the three lumens at the fourth position 2640, the average bending stiffness at the fourth position 2640 can be (and was observed to be) higher than the average bending stiffness at the first, second, and third positions 2610, 2620, 2630, respectively. For example, at the third position 2630, there are four lumens compared to the three lumens at the fourth position 2640. Thus, the bending stiffness at the fourth position 2640 is observed to be greater than that at the third position 2630.

Further, the average outer diameter at the third position 2630 is about 0.9 mm, while the average diameter at the fourth position 2640 is about 1.15 mm. Thus, based on the difference in the outer diameters and the number of lumens, the average bending stiffness at the fourth position 2640 is observed to be greater than that at the third position 2630. As noted elsewhere herein, the increased stiffness can also yield greater columnar strength in that location, aiding pushability.

Increased bending stiffness in a proximal region can correspond to increased buckling strength (e.g., longitudinal buckling strength, e.g., relative to so-called Euler buckling) in the proximal region. Increased buckling strength, in turn, can correspond to increased pushability of the IVL catheter 2600, as relatively higher longitudinal loads can be applied to the IVL catheter 2600 in regions with higher buckling strength. As a portion of the IVL catheter 2600 is fed into a tortuous vascular system, the longitudinal force necessary at or near an entry to the vasculature to advance the device incrementally increases in correspondence with the length of the portion already inserted. Thus, by providing a thicker wall thickness near the proximal portion of the IVL catheter 2600, pushability can increase. Further, a relatively lower bending stiffness in a distal region of the IVL catheter 2600, provided by the tapered wall thickness, can allow relatively distal regions of the IVL catheter 2600 to more easily track through the tortuosity of a vasculature system.

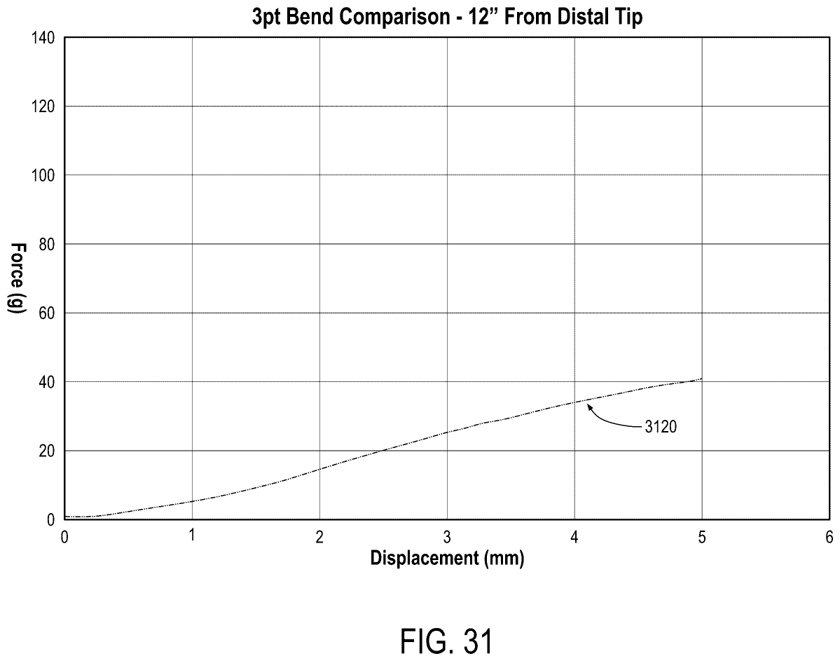

Referring now to FIG. 31, test results at the fifth position 2650, which is located at about 12 inches from the distal tip (e.g., the distal tip 218) of the IVL catheter 2600 are described. The force-displacement curve 3120 shows average test results for IVL catheters with 20 mm balloons. The fifth position 2650 is positioned proximally of the rapid exchange port between the rapid exchange port (e.g., the RX port 406 of FIGS. 12C, 13A, and 13B) and the distal tip (e.g., the distal tip 404) of the hypotube. In this region, there are three lumens for wire conductors 300 in the polymer tube. Two lumens may float within the inside of the polymer tube 430 and the saline solution passes along the two lumens. In another configuration, no lumens are provided for the wire conductors 300 and they float within the lumen for the saline solution. In one case, the two lumens for the wire conductors 300, when included in the device, may be attached to the inside surface of the outer wall of the polymer tube 430. In each case, the inside of the polymer tube 430 can remain empty. Toward the rapid exchange port, the thickness of the outer wall of the polymer tube 430 can become thicker and thicker so that the empty space becomes solid except the two lumens (when included for the wire conductors 300) and one lumen for the saline solution proximal to the rapid exchange port.

Based on the force-displacement curve 3010, the average bending stiffness is above 10.5 g/mm over the whole range for IVL catheters with the 12 mm balloons, while the average slope is about 8 g/mm based on the test results from the force-displacement curve 3020.

Based on the test results for IVL catheters with the 12 mm balloons, the average bending stiffness is about 10.5 g/mm, and the standard deviation is about 0.21 g/mm with the sample size of 3. Thus, according to the statistical analysis, the range of the bending stiffness is from 10.26 g/mm to 10.73 g/mm with the 95% confidence interval. With the 99% confidence interval, the range of the bending stiffness is from 9.08 g/mm to 11.9 g/mm 2. On the other hand, the maximum bending stiffness was 10.72 g/mm and the minimum bending stiffness was 10.32 g/mm based on the actual test results.

At the fifth position 2650, the polymer tube 430 has an outer wall 434 with the inside being empty. The two wire conductors 300 are surrounded by a respective internal polymer outer wall 434, which is separate from the inside surface of the outer wall 434 of the polymer tube 430. In this case, the two wire conductors 300 float within the inside space and the saline solution passes along the two wire conductors 300. The thickness of the outer wall 434 is about 0.00425″, while the thickness of the outer wall 434 of the polymer tube 430 at the fourth position 2640 is about the diameter of the polymer tube 430. Thus, the bending stiffness at the fourth position 2640 was observed to be much greater than the bending stiffness at the fifth position 2650.

The following TABLE 3 shows a summary of the 3-point bending tests of IVL catheters with 20 mm balloons. These results illustrated improved bending stiffness of the polymer tube and so the system is achieved without a mandrel extending from the hypotube to the balloon. The average bending stiffness of the IVL catheter as shown below in TABLE 3 may be measured at positions as described above, at positions.

TABLE 3

| | | Bending Stiffness of |
|---|---|---|
| | | Device with a 20 mm |
| Position | Statistical Measure | Balloon (g/mm) |
| 1^{st} | Mean | 8.7 |
| Position | Standard Deviation | 0.83 |
| | 95% CI | 7.76 to 9.64 |
| | 99% CI | 7.47 to 9.93 |
| | Measured Max | 9.68 |
| | Measured Min | 8.10 |
| 2^{nd} | Mean | 9.6 |
| Position | Standard Deviation | 0.20 |
| | 95% CI | 9.37 to 9.83 |
| | 99% CI | 9.30 to 9.90 |
| | Measured Max | 9.85 |
| | Measured Min | 9.50 |
| 3^{rd} | Mean | 8.5 |
| Position | Standard Deviation | 0.90 |
| | 95% CI | 7.48 to 9.52 |
| | 99% CI | 7.16 to 9.84 |
| | Measured Max | 9.20 |
| | Measured Min | 7.46 |
| 4^{th} | Mean | 15.1 |
| Position | Standard Deviation | 1.08 |
| | 95% CI | 13.88 to 16.32 |
| | 99% CI | 13.49 to 16.71 |
| | Measured Max | 16.39 |
| | Measured Min | 14.43 |
| 5^{th} | Mean | 10.5 |
| Position | Standard Deviation | 0.21 |
| | 95% CI | 10.26 to 10.73 |
| | 99% CI | 9.08 to 11.92 |

| | |
|---|---|
| Measured Max | 10.72 |
| Measured Min | 10.32 |

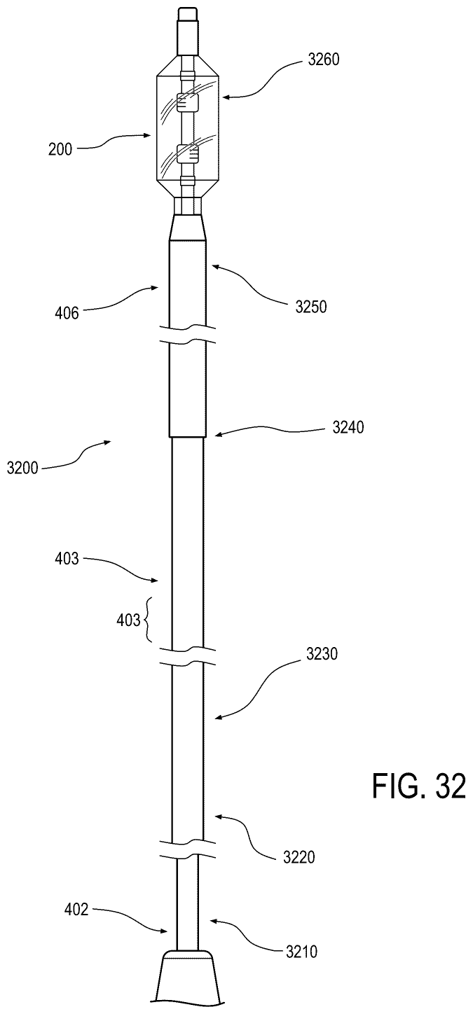

Now, cross-sectional views are described at different locations at the IVL catheter 3200 according to various aspects of the present disclosure. The disclosures and teachings presented here in relation to the IVL catheters of FIGS. 1-31 are also applicable to the IVL catheter 3200. FIG. 32 illustrates cutting positions 3210, 3220, 3230, 3240, 3250 and 3260, e.g., positions where cross-sectional views are taken as described below. The first position 3210 is located at the proximal portion of the hypotube 402; the second position 3220 is located at a bonded section 403 (e.g., FIGS. 12B1-12B3) where the hypotube 402 and the polymer tube 430 overlap; the third position 3230 is located at the proximal portion of the polymer tube 430; the fourth position 3240 is located at a distal portion of the rapid exchange port 406; the fifth position 3250 is located between the rapid exchange port 406 and the balloon 200; and the sixth position 3260 is located at the balloon 200. The cross-sectional structure between two adjacent positions may be considered to be progressively transitioned from the cross-sectional structure from one position to the other position. In each cross-sectional view, diameters of lumens and tubes, thicknesses of walls, structures, constructions, and bonds, which contribute to enhanced deliverability and pushability may be described below.

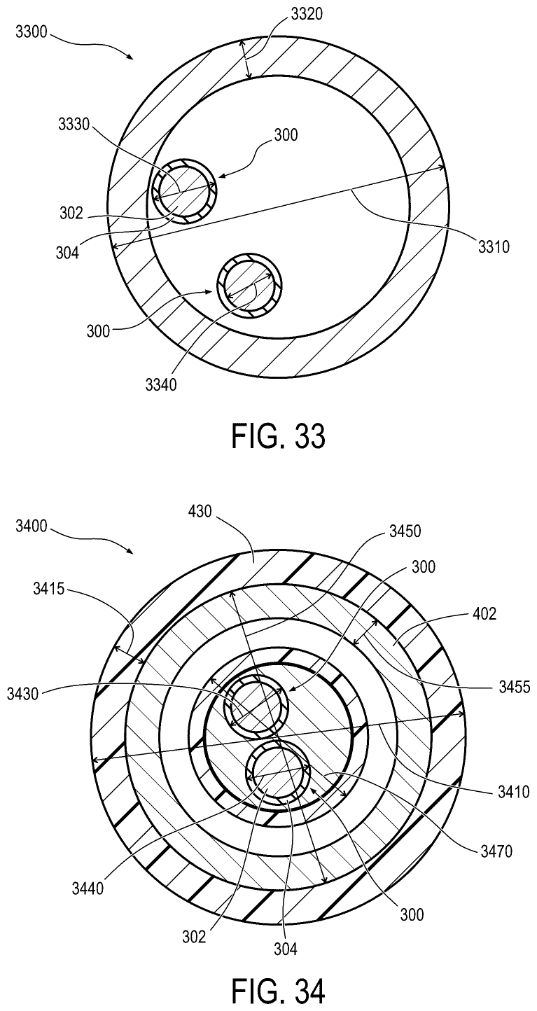

The cross-sectional view 3300 at the first position 3210 is illustrated in FIG. 33. At the first position 3210, the hypotube 402 is cut in a direction perpendicular to the longitudinal direction. The hypotube 402 may be made of stainless steel or any compatible bio-friendly metal or plastic, which provides strong columnar strength and pushability characteristics to the IVL catheter 3200. Due to the strong columnar strength, the hypotube 402 may form a circle in the cross-sectional view 3200. While the hypotube 402 is illustrated as being generally circular in cross-section, it is understood that the hypotube 402 can have other cross-sectional configurations, such as oval, etc. The diameter 3310 of the hypotube 402 is about 0.0347 inches. The thickness 3320 of the hypotube 402 is about 0.0037 inches. Thus, the internal diameter of hypotube 402 is about 0.0273 inches. From the proximal tip to the distal tip of the hypotube 402, the hypotube may maintain the substantially consistent structure. Alternatively, in other configurations the hypotube 402 can taper proximally to distally or have other non-consistent structure configurations.

The inside space of the hypotube 402 is generally empty so that wire conductors 300, pass throughout the inside space. Saline solution also passes through the inside space along the wire conductors 300. The illustrated wire conductors 300 include conductive cores 302 and insulating covers 304. The insulating covers 304 may have substantially similar dimensional properties. For example, the diameters 3330 and 3340 of the insulating cover 304 are about 0.0051 inches and 0.0050 inches, respectively.

Referring now to the second position 3220, the hypotube 402 and the polymer tube 430 overlap, as illustrated in cross-section 3400 shown in FIG. 34. Catheter deliverability involves a balance between a low profile and a proper material construction balance for optimized columnar strength (pushability) and flexibility. With reference to FIGS. 32 and 34, the bonded section 403 at the second position 3220 is where the IVL catheter 3200 transitions from the hypotube 402 to the polymer tube 430. To reduce kinking at the junction of the hypotube 403 and the polymer tube 420, and limit the possibility that the sharp distal tip (e.g., the distal tip 404) of the hypotube 402 may rupture or tear the insulation cover 3330 and 3340 of the wire conductors 300, the outer surface of the hypotube 403 in the bonded section 403 is surrounded by an outer polymer tube 420 (e.g., FIGS. 12B2 and 12B3 and the outer polymer tube 420′ of FIGS. 12B3). The thickness 3415 of the outer polymer tube 420 is about 0.0035 inches.

The outer diameter 3410 of the bonded section 403 is about 0.0407 inches. By subtracting two thicknesses 3415 of the outer polymer tube 420 from the outer diameter 3410, the outer diameter of the hypotube 402 is about 0.0337 inches, while the outer diameter 3454 is identified as 0.0306 inches, which is smaller than 0.0337 inches. It is noted that due to the small sizes, the overall measurements might not be exactly the same around the perimeter of the IVL catheter 3200 but provide general representative dimensions of the structural components at each position 3210-3260. In an aspect, the outer diameter 3450 of the hypotube 402 may smaller than the diameter 3310 of the hypotube 402 of FIG. 33 at the first position 3210. In this regard, the hypotube 402 may decrease in size toward the distal tip (e.g., the distal tip 404) thereof, thereby obtaining the low profile in the bonded section 403.

The bonded section 403 may be about 1.5 inches long, and the outer polymer tube 420 may start covering the hypotube 402 before the bonded section 403 and end covering after the bonded section 403. The thickness of the outer polymer tube 420 may be smaller towards a proximal end and become thicker in the middle section and distal end of the bonded section 403. The overall outer diameter 3410 in the bonded section 403 may be smaller than the outer diameter of conventional IVL catheters in the market by about 0.003 inches because of the varying thickness of the outer polymer tube 420. This varying thickness of the outer polymer tube 420 may provide sufficient columnar strength, thereby increasing pushability for the distal portion of the IVL catheter 3200. Further, by covering the bonded section 403 and the distal portion of the distal tip (e.g., the distal tip 404 and surrounding area) of the hypotube, the outer polymer tube 420 may function as a strain relief that allows for a maximization of transfer of axial push force while minimizing kinking and reducing overall profile compared to other design options.

The polymer layer 420 overlaps the hypotube 402 in the bonded section 403. The two wire conductors 300 enter into the polymer tube 430 at the proximal portion of the polymer tube 430. Within the polymer tube 430, the insulating covers 304 have the outer diameters 3430 and 3440, of which both are about 0.0051 inches. Saline solution may pass into an inside of the polymer tube 430 along with the wire conductors 300. The outer diameter of the polymer tube 430 can be smaller than the inner diameter of the hypotube 403. The inner diameter of the hypotube 403 may be calculated by subtracting two times the thickness of the hypotube 403 from the outer diameter of the hypotube 403, 0.0306-2*0.0040, to be about 0.0226 inches, and based on the outer diameters 3430 and 3440 of the insulating covers 304, the outer diameter of the polymer tube 430 within the bonded section 403 may be greater than 0.013 inches and less than 0.22 inches. Further, the thickness of the polymer tube 430 is similar to or smaller than the thickness of the outer polymer tube 420.

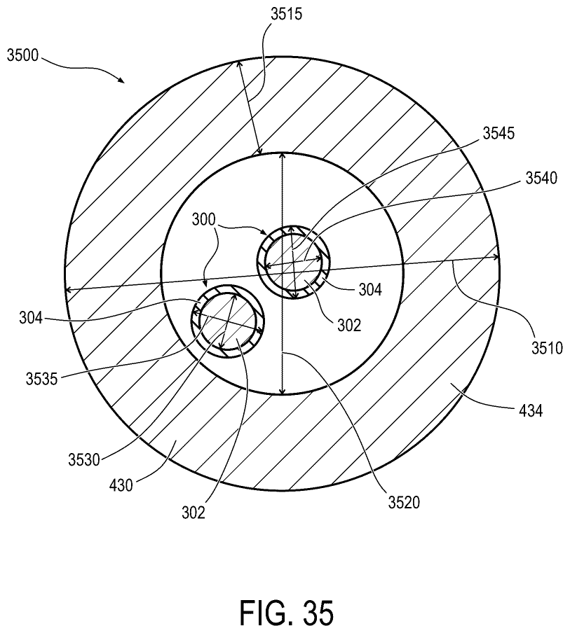

Distal to the ending section of the outer polymer tube 420 and proximal to the polymer tube 430 after the bonded section 403, illustrated in FIG. 35 is the cross-sectional view 3500 at the third position 3230. The outer polymer tube 420 is no longer shown in the cross-sectional view 3500. Instead, the thickness of the polymer tube 430 is thickened toward the distal portion thereof. Specifically, the outer diameter 3510 of the polymer tube 430 is about 0.0409 inches, and the inner diameter of its inner space or lumen 432 is about 0.0228 inches. Thus, the thickness 3515 of its outer wall 434 is about 0.0091 inches, which is at least two times thicker than the thickness 3415 of the outer polymer tube 420 of FIG. 34. While this is the case in one configuration, the thickness 3515 of the outer wall 43 can be less than two times thicker than the thickness 3415 of the outer polymer tube 420 or greater than two times thicker than the thickness 3415 of the outer polymer tube 420.

The inner space or lumen 432 of the polymer tube 432 is empty except for the wire conductors 300. The outer diameters 3530 and 3540 of the insulating cover 304, and so the outer diameter of the wire conductors 300, are substantially similar to the diameters 3330, 3340, 3430 and 3440, respectively. Generally, there is a lumen for a supporting structure in conventional catheters at a position corresponding to the third position 3230. The supporting structure may be a support mandrel, which provides sufficient stiffness so as to provide pushability and deliverability through coronary vessels. The presence of the support mandrel may cause flexibility discrepancy between bending in the vertical plane and the horizontal plane. On the other hand, however, according to aspects of the present disclosure, there is no supporting structure (e.g., a support mandrel) in the IVL catheter 3200. For instance, there is no mandrel extending from the hypotube 402 to the balloon 200 or any portion of the distance between the hypotube 402 and the balloon 200. Instead, the thickness of the outer wall 434 of the polymer tube 430 is thickened to provide sufficient pushability and deliverability. Stated another way, improved bending stiffness of the polymer tube is achieved without a mandrel extending from the hypotube to the balloon.

FIG. 36A illustrates a perspective view of the rapid exchange port 406 in the top and FIG. 36B a cross-section view along the A-A direction in the proximal portion of the rapid exchange port 406. The shape of the polymer tube 430 forms a longitudinal groove 436 prior to the rapid exchange port 406 so that the outer surface can form a smooth transition from the circular top shape to a dimple shape, recess, groove, or an upside down circular shape on the top thereof prior to the rapid exchange port 406. The shape of the groove 436 or dimple may be circular, elliptic, oval, or any other curve shapes that can be used to smoothly receive a guidewire into the rapid exchange port toward the inside of the polymer tube.

The outer diameter of the polymer tube 430 in a horizontal direction with respect to the cross-sectional view canto be similar to that of the polymer tube 430 before the groove 436 is formed. On the other hand, the outer diameter of the polymer tube 430 in a vertical direction can be shorter than that in the horizontal direction. In the cross-sectional view, there are two top portions 3680 and 3685. Near the rapid exchange port 406, the top two portions 3680 and 3685 are connected together so that the space under the connected portion forms an opening or lumen inside the polymer tube 430. Stated another way, while the polymer tube 430 includes the groove 436 to aid with positioning a guidewire into the rapid exchange port 406, the lumen 432 still accommodates the wire conductors 300 and saline solution. Through the lumen or the exchange port 406, the guidewire may be inserted into the polymer tube 430.

The rapid exchange port 406 may provide an access for the guidewire or other interventional tools. The rapid exchange port 406 may comprise a 63D Pebax® tube with a high density polyethylene (HDPE) such as Rezilok® and has an inner surface that may be lined with 63D Pebax®. The lumen for the guidewire may extend distally through the polymer tube 430.

In an aspect, the insulating cover 304 for the wire conductors 300 may be fixed to the inside surface of the outer wall 434 of the polymer tube 430, as illustrated in the cross-sectional view of FIG. 36B. In another aspect, the wire conductors 300 may be incorporated into the outer wall 434 of the polymer tube 430. In such a case, at the rapid exchange port 406, the polymer tube 430 can have three lumens and one comparatively large inside space or lumen. Two of the three lumens are for wire conductors 300, one lumen for a guidewire, and one large inside space used for transporting the saline solution.

It is noted that a thicker wall may provide a uniform kink resistance in all directions compared to a guidewire that would be bias to the position of the guidewire. In this regard, as the outer wall 434 thickens, the inside space becomes smaller and smaller. Thus, distal to the rapid exchange port 406, the inside space becomes another lumen. At this point, the polymer tube 430 includes four lumens. The fourth position 3240 is located at a position where the four lumens are formed within the polymer tube 430, as illustrated in FIG. 36C. For example, the fourth position 3240 is located 3.5 cm distally from the rapid exchange port 406.

As illustrated, a guidewire lumen 438 and a saline solution lumen 432 are formed in the wall 434 of the polymer tube 430. The wire conductors 300 are also disposed within the wall 434 of the polymer tube 430, optionally with lumens 3622, 3624. The diameter 3610 of the polymer tube 430 is the outer diameter 0.0499 inches. The diameters 3620 and 3625 of one lumen 3622 for one wire conductor 300 in two directions are 0.0068 and 0.0072 inches, respectively, and the diameters 3630 and 3635 of another lumen 3624 for the other wire conductor 300 in two directions are 0.0069 and 0.0072 inches in two directions, respectively. In other configurations, the diameters for each lumen 3622, 3624 are the same or substantially the same in one or two direction. The diameter 3650 of the lumen 438 for the guidewire is about 0.0151 inches, and the closest distance 3652 to the outer surface of the polymer tube 430 is about 0.0047 inches. Now, with regard to the saline solution, the diameter 3660 of the corresponding lumen 432 is about 0.0111 inches and the closest distance 3662 to the outer surface of the polymer tube 430 is about 0.0110 inches.

Due to the solidness within the polymer tube 430, the wire conductors 300 cannot freely float therein but are rather fixed therein. That also provides columnar strength to the polymer tube 430 to enhance pushability and deliverability. Further, as described above, the bending stiffness near the rapid exchange port is the highest among others. This highest bending stiffness may be based on the solid characteristics of the polymer tube 430 at the fourth position 3140. In other words, the bending stiffness at the rapid exchange port may be largely from the material and solid configuration of the polymer tube 430. In other words, a majority of the bending stiffness at the polymer tube 430 or at the balloon 200 is provided by a first polymer material forming a wall of the polymer tube 430 or a second polymer material forming a wall of the elongate member 220. The first polymer material may be same as or different from the second polymer material.

The construction of the polymer tube 430 may be changed as the position becomes close to the balloon 200 (e.g., FIG. 32). For example, illustrated in FIG. 37 is the cross-sectional view 3700 at the fifth position 3250, which is located about 8.5 inches from the distal tip (e.g., the distal tip 218 of FIG. 6) of the IVL catheter 3200 between the rapid exchange port and the distal tip 218 of the polymer tube 430. The outer diameter 3710 of the polymer tube 430 is about 0.0467 inches in one direction and the outer diameter 3715 of the polymer tube 430 is about 0.0440 inches in another direction. The overall outer diameter of the polymer tube 430 becomes smaller than that at the fourth position 3140 near the rapid exchange port 406. The thickness of the outer wall 434 of the polymer tube 430 is about 0.0036 inches.

As the lumen 432 for the saline solution becomes wider and fills the inside of the polymer tube 430, the polymer tube 430 has two lumens at the fifth position 3250. One lumen is provided for the guidewire, i.e., the guidewire lumen 438 disposed within a guidewire tube 450 within an inner polymer tube 452, and the two wire conductors 300 are disposed in the lumen 432 through which saline solution flows to and from the balloon 200. Optionally the guidewire tube 450 and the inner polymer tube 452 can be a single tube rather than two tubes. The two wire conductors 300, and optionally the polymer tube(s) forming the guidewire lumen 438, can float and freely move within the saline solution bounded by the outer wall of the polymer tube 430 and the inner polymer tube 452. Due to this construction, the bending stiffness near the fifth position 3250 is less than the bending stiffness at the rapid exchange port. Further, based on this construction, the IVL catheter 3200 may navigate various curvatures of coronary vessels to reach calcified region, thereby increasing deliverability of the IVL catheter 3200.

The inner and outer diameters 3753 and 3753 of the guidewire lumen 438 is about 0.156 inches and 0.0179 inches, and the thickness 3754 of tube forming the guidewire lumen 438 is about 0.0015 inches. The inner polymer tube 452 surrounds the guidewire tube 450 and forms the guidewire lumen 438, with an outer diameter of the inner polymer tube 452 being 0.0273 inches. As described, the inner polymer tube 452 surrounds the guidewire lumen 438. The outer diameter 3750 of the combination of the inner polymer tube 452 and the guidewire lumen 438 is about 0.0273 inches and the thickness 3755 of the inner polymer tube 452 is about 0.0044 inches.

The outer diameters 3720 and 3730 of two wire conductors 300 are about 0.0051 and 0.0052 inches, respectively, while both the thicknesses 3725 and 3735 of the insulating cover 304 are about 0.0010 inches.

Based on this construction, the saline solution is contained between the outer wall of the inner polymer tube 452 and the inner wall of the polymer tube 430 and the insulating cover 304 of the wire conductors 300 in this section of the IVL catheter 3200.

Since the thickness of the wall for the guidewire lumen 438 is thicker than the thickness of the inner walls for the electrode lumens, pushability of the IVL catheter 3200 may be maintained in this section of the IVL catheter 3200, while providing a smaller bending stiffness than that in a relatively more proximal region, e.g., at or near the rapid exchange port.

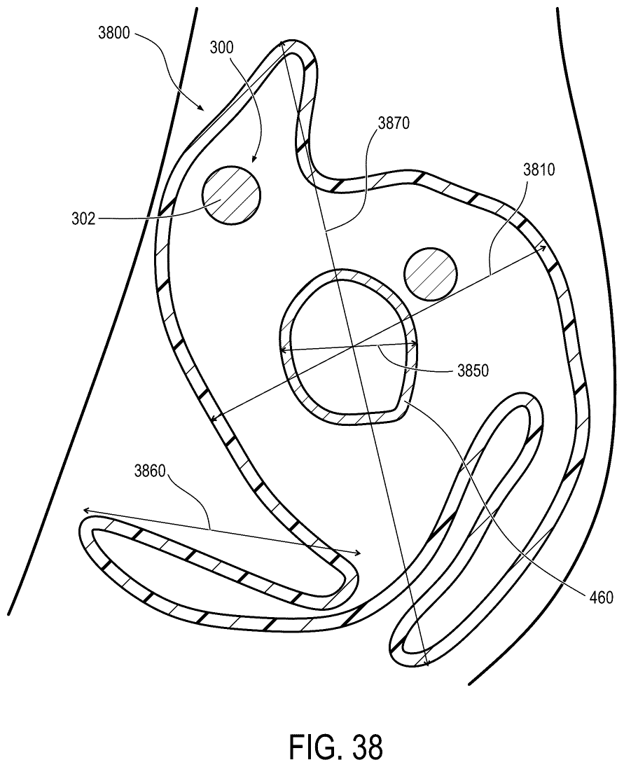

Now referring to FIG. 38, construction of the IVL catheter 3200 at the sixth position 3260 is illustrated in the cross-sectional view 3800. The sixth position 3260 is positioned at or near the center of the balloon 200. An elongate member 460 (e.g., the elongate member 220 of FIGS. 6, 7B, 9-11, 12D, 14A, 14B, and 17A) may be extended from the distal tip of the polymer tube 430. The distal tip of the polymer tube 430 (e.g., the outer member 230 of FIG. 6) is tapering off in a distal direction. The elongate member 220 may be received within and extended distally from the distal tip of the tapering portion of the polymer tube 430. The elongate member 220 may have a smaller cross-sectional profile than the polymer tube 430, meaning that the outer diameter of the elongate member 460 is about 0.0177 inches, smaller than the outer diameters of the polymer tube 430. An electrode support member ES (ES or ES' of FIGS. 6, 7B, 9, 11, 12D, 14A, 14B, and 15A-15D) may be positioned around the elongate member 220. For example, the outer diameter 3180 of the ES is about 0.0483 inches. Due to the smaller crossing profile of the elongate member 460, less push force is required in this region. In an aspect, the guidewire may reach the sixth position 3260 or not. When the guidewire reaches the sixth position 3260, the guidewire may be positioned within the elongate member 460.

As described above, the distal tip (e.g., the distal tip 218) of the IVL catheter 3200 has a tapering end. Thus, the crossing profile of the distal tip also has a smaller profile than any other regions of the IVL catheter 3200, thereby enhancing the pushability and deliverability of the IVL catheter 3200.

The pleated portion 3860 of the balloon may be about 0.0348 inches long, and the longest diameter 3870 of the combination of the elongate member 460, the ES, and the pleated portion may be about 0.0817 inches.

As described above, the crossing profile of the IVL catheter 3200 varies at different positions 3210-3260 as illustrated in FIGS. 33-38. Varying thickness of the outer wall of the polymer tube, varying sizes of cross-sectional portions, presence of solid inside of the polymer tube, and varying bending stiffness may enhance pushability and deliverability of the IVL catheter 3200.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

Term “majority” is intended to mean more than 50% and “largely” also means more than 50%. They may mean more than 60% or 70% and less than or equal to 100%.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Where possible, like numbering of elements have been used in various figures. Furthermore, multiple instances of an element and or sub-elements of a parent element may each include separate letters appended to the element number. For example, two instances of a particular element “10” may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element. For example, two alternative exemplary embodiments of a particular element may be labeled as “10A” and “10B”. In that case, the element label may be used without an appended letter (e.g., “10”) to generally refer to all instances of the alternative embodiments or any one of the alternative embodiments.

Following are some further example configurations of disclosed principles. These are presented only by way of example and are not intended to limit the scope of the disclosed principles in any way. Further, any example configuration can be combined with one or more other of the example configurations.

Configuration 1. An intravascular lithotripsy (“IVL”) catheter having a shaft comprising a proximal section extending from a proximal hub to a distal end of a hypotube associated with the proximal hub; a mid-section comprising a polyimide conduit extending distally from the distal end of the hypotube to a proximal end of an IVL balloon or enclosure; and a distal section extending distally from the proximal end of the IVL balloon or enclosure, wherein the proximal section includes an overlapping bonded region comprising a polymer wall surrounding the hypotube, wherein the polymer wall comprises a first thickness, and wherein the mid-section comprises a proximal end adjacent to the distal end of the hypotube, and comprising the polymer wall surrounding the polyimide conduit, the polymer wall having a second wall thickness that is greater than the first thickness of the polymer wall.

Configuration 2. The IVL catheter of configuration 1, wherein the second wall thickness is between 10% and 50% greater than the first thickness.

Configuration 3. The IVL catheter of any of configurations 1-2, wherein the second wall thickness is between 50% and 60% greater than the first thickness.

Configuration 4. The IVL catheter of any of configurations 1-3, wherein the second wall thickness is between 60% and 70% greater than the first thickness.

Configuration 5. The IVL catheter of any of configurations 1-4, wherein the second wall thickness is more than 70% greater than the first thickness.

Configuration 6. The IVL catheter of any of configurations 1-5, wherein the second wall thickness decreases in the distal direction.

Configuration 7. The IVL catheter of any of configurations 1-6, wherein the second wall thickness at the proximal end of the mid-section is about 0.009 inches.

Configuration 8. The IVL catheter of any of configurations 1-7, wherein the second wall thickness at the distal end of the midsection is about 0.0038 inches.

Configuration 9. The IVL catheter of any of configurations 1-8, wherein the second wall thickness at the distal end of the midsection is about 0.0038 inches.

Configuration 10. The IVL catheter of any of configurations 1-9, further comprising a maximum outer diameter in the overlapping bonded region of about 0.044 inches.

Configuration 11. The IVL catheter of any of configurations 1-2, further comprising a maximum outer diameter in the overlapping bonded region of about 0.041 inches.

Configuration 12. The IVL catheter of any of configurations 1-10, further comprising a maximum outer diameter in the overlapping bonded region of about 0.0435 inches.

Configuration 13. The IVL catheter of any of configurations 1-11, further comprising a maximum outer diameter in the overlapping bonded region within a range of about 0.041 inches to about 0.044 inches.

Configuration 14. The IVL catheter of any of configurations 1-12 wherein the polymer wall of the proximal section comprises 72D Pebax®.

Configuration 15. The IVL catheter of any of configurations 1-13, wherein the polymer wall of the mid-section comprises 72D Pebax®.

Configuration 16. The IVL catheter of any of configurations 1-14, wherein the polymer wall of the mid-section comprises 72D Pebax®.

Configuration 17. The IVL catheter of any of configurations 1-15, wherein the polymer wall of the mid-section is configured to provide uniform kink resistance in any bending or flexing direction within the midsection.

Configuration 18. The IVL catheter of any of configurations 1-16, wherein the polymer wall of the mid-section is configured to provide increased columnar strength and pushability.

Configuration 19. The IVL catheter of any of configurations 1-17, wherein the polyimide tube is configured to provide increased columnar strength and pushability. Configuration 20. The IVL catheter as shown and described.

Configuration 21. A method for conducting intravascular lithotripsy using any one or more of the IVL catheters of configurations 1-20.

Configuration 22. An intravascular lithotripsy (IVL) catheter assembly, comprising:
a hypotube; a polymer tube connected to a distal portion of the hypotube; and a balloon bonded to a distal portion of the polymer tube, wherein the hypotube, the polymer tube, and the balloon are configured in size and shape to allow at least a portion of the IVL catheter assembly to be inserted into a coronary vessel, and wherein a bending stiffness of the polymer tube is less than or equal to 20 g/mm.

Configuration 23. The IVL catheter assembly of configuration 22, wherein the bending stiffness is less than or equal to 16.7 g/mm.

Configuration 24. The IVL catheter assembly of any of configurations 22 and 23, wherein the bending stiffness is measured at a position within a range from the balloon to 12 inches from a distal tip of the IVL catheter assembly.

Configuration 25. The IVL catheter assembly of any of configurations 22-24, wherein the bending stiffness is an average of forces, which displace the polymer tube by 5 mm.

Configuration 26. The IVL catheter assembly of any of configurations 22-25, wherein the polymer tube forms a port, which is to receive a guidewire, in a middle portion thereof.

Configuration 27. The IVL catheter assembly of any of configurations 22-26, wherein a bending stiffness measured at the port is higher than a bending stiffness measured at 12 inches from a distal tip of the IVL catheter assembly.

Configuration 28. The IVL catheter assembly of any of configurations 22-26, wherein a wall of the polymer tube comprises two or more lumens distal to the port.

Configuration 29. The IVL catheter assembly of any of configurations 22-28, wherein the two or more lumens includes lumens for two wire conductors, saline solution, and the guidewire to pass up to a portion, to which the balloon is bonded.

Configuration 30. The IVL catheter assembly of any of configurations 22-29, wherein an outer wall of the polymer tube thickens from a distal tip of the hypotube.

Configuration 31. The IVL catheter assembly of any of configurations 22-30, wherein the polymer tube has no support structure other than a material of the polymer tube.

Configuration 32. An intravascular lithotripsy (IVL) catheter assembly comprising: a hypotube; a polymer tube connected to a distal portion of the hypotube; and a balloon bonded to a distal portion of the polymer tube, wherein the hypotube, the polymer tube, and the balloon are configured in size and shape to allow at least a portion of the IVL catheter assembly to be inserted into a coronary vessel, wherein the polymer tube forms a port, which is to receive a guidewire, in a middle portion thereof, and wherein, distal to the port and proximal to the balloon, the polymer tube forms two or more lumens.

Configuration 33. The IVL catheter assembly of configuration 32, wherein an outer wall of the polymer tube thickens from a distal tip of the hypotube to the port.

Configuration 34. The IVL catheter assembly of any of configurations 32-33, wherein an outer wall of the polymer tube enlarges in a dimension transverse to a longitudinal axis and includes the two or more lumens.

Configuration 35. The IVL catheter assembly of any of configurations 32-34, wherein the two or more lumens are selected from a first lumen for a first wire conductor, a second lumen for a second wire conductor, a third lumen for saline solution, and a fourth lumen for the guidewire.

Configuration 36. The IVL catheter assembly of any of configurations 32-35, wherein, proximal to the port, the polymer tube forms an empty space therein.

Configuration 37. The IVL catheter assembly of any of configurations 32-36, wherein, a size of one lumen of the two or lumens is increased from the port toward a distal portion of the polymer tube.

Configuration 38. The IVL catheter assembly of any of configurations 32-37, wherein the one lumen is for saline solution to pass therethrough.

Configuration 39. The IVL catheter assembly of any of configurations 32-38, wherein the polymer tube has no support structure other than a material thereof.

Configuration 40. The IVL catheter assembly of any of configurations 32-39, further comprising:
an outer polymer tube overlapping the distal portion of the hypotube and a portion distal to a distal tip of the hypotube.

Configuration 41. An intravascular lithotripsy (IVL) catheter assembly comprising: a hypotube; a polymer tube connected to a distal portion of the hypotube; and a balloon bonded to a distal portion of the polymer tube, wherein the hypotube, the polymer tube, and the balloon are configured in size and shape to allow at least a portion of the IVL catheter assembly to be inserted into a coronary vessel, and wherein the polymer tube forms at least two lumens for two wire conductors, and wherein the two lumens are incorporated within an outer wall of the polymer tube.

Configuration 42. An intravascular lithotripsy (IVL) catheter assembly, comprising: a hypotube; a polymer tube connected to a distal portion of the hypotube; and a balloon bonded to a distal portion of the polymer tube, wherein the hypotube, the polymer tube, and the balloon are configured in size and shape to allow at least a portion of the IVL catheter assembly to be inserted into a coronary vessel, and wherein a bending stiffness of the polymer tube is less than or equal to 20 g/mm without a mandrel disposed between a distal end of the hypotube and a proximal end of the balloon.

Configuration 43. The IVL catheter assembly of configuration 42, wherein the bending stiffness is less than or equal to 9.6 g/mm at a position of 3 inches from a distal tip of the IVL catheter assembly.

Configuration 44. The IVL catheter assembly of any of configurations 42 and 43, wherein the bending stiffness is less than or equal to 8.5 g/mm at a position of 6 inches from a distal tip of the IVL catheter assembly.

Configuration 45. The IVL catheter assembly of any of configurations 42-44, wherein the bending stiffness is less than or equal to 10.5 g/mm at a position of 12 inches from a distal tip of the IVL catheter assembly.

Configuration 46. The IVL catheter assembly of any of configurations 42-45, wherein the bending stiffness is less than or equal to 15.1 g/mm at a rapid exchange port of the IVL catheter assembly.

Configuration 47. The IVL catheter assembly of any of configurations 42-46, wherein the bending stiffness is less than or equal to 8.10 g/mm to 9.68 g/mm at a position of the balloon.

Configuration 48. The IVL catheter assembly of any of configurations 42-47, wherein the bending stiffness is less than or equal to 9.50 g/mm to 9.85 g/mm at a position of 3 inches from a distal tip of the IVL catheter assembly.

Configuration 49. The IVL catheter assembly of any of configurations 42-48, wherein the bending stiffness is less than or equal to 7.46 g/mm to 9.20 g/mm at a position of 6 inches from a distal tip of the IVL catheter assembly.

Configuration 50. The IVL catheter assembly of any of configurations 42-49, wherein the bending stiffness is less than or equal to 10.32 g/mm to 10.72 g/mm at a position of 12 inches from a distal tip of the IVL catheter assembly.

Configuration 51. The IVL catheter assembly of any of configurations 42-50, wherein the bending stiffness is less than or equal to 14.43 g/mm to 16.39 g/mm at a rapid exchange port of the IVL catheter assembly.

Configuration 52. An intravascular lithotripsy (IVL) catheter assembly, comprising: a hypotube; a polymer tube connected to a distal portion of the hypotube; and a balloon bonded to a distal portion of the polymer tube, wherein the hypotube, the polymer tube, and the balloon are configured in size and shape to allow at least a portion of the IVL catheter assembly to be inserted into a coronary vessel, and wherein a bending stiffness of the polymer tube is less than or equal to about 15 g/mm at a position, where a majority of the bending stiffness is provided by a first polymer forming a wall of the polymer tube.

Configuration 53. The IVL catheter assembly of configuration 52, wherein the bending stiffness at the position, which is disposed 3 inches from a distal tip of the IVL catheter assembly, is less than or equal to 9.6 g/mm.

Configuration 54. The IVL catheter assembly of any of configurations 52 and 53, wherein the bending stiffness at the position, which is disposed 6 inches from a distal tip of the IVL catheter assembly, is less than or equal to 8.5 g/mm.

Configuration 55. The IVL catheter assembly of any of configurations 52-54, wherein the bending stiffness at the position, which is disposed 12 inches from a distal tip of the IVL catheter assembly, is less than or equal to 10.5 g/mm.

Configuration 56. The IVL catheter assembly of any of configurations 52-55, wherein the bending stiffness at the position, which is disposed at a rapid exchange port of the IVL catheter assembly, is less than or equal to 15.1 g/mm.

Configuration 57. The IVL catheter assembly of any of configurations 52-56, wherein the bending stiffness at the position, which is disposed 3 inches from a distal tip of the IVL catheter assembly, is less than or equal to 9.50 g/mm to 9.85 g/mm.

Configuration 58. The IVL catheter assembly of any of configurations 52-57, wherein the bending stiffness at the position, which is disposed 6 inches from a distal tip of the IVL catheter assembly, is less than or equal to 7.46 g/mm to 9.20 g/mm.

Configuration 59. The IVL catheter assembly of any of configurations 52-58, wherein the bending stiffness at the position, which is disposed 12 inches from a distal tip of the IVL catheter assembly, is less than or equal to 10.32 g/mm to 10.72 g/mm.

Configuration 60. The IVL catheter assembly of any of configurations 52-59, wherein the bending stiffness at the position, which is disposed at a rapid exchange port of the IVL catheter assembly, is less than or equal to 14.43 g/mm to 16.39 g/mm.

Configuration 61. The IVL catheter assembly of any of configurations 52-60, wherein the bending stiffness at the position, which is disposed at the balloon of the IVL catheter assembly, is less than or equal to 8.10 g/mm to 9.68 g/mm.

The description of disclosed principles and their applications as set forth herein is illustrative and is not intended to limit the scope of this disclosure or any claims presented herein or at any time throughout prosecution. Each reference incorporated herein by reference is hereby, and is intended to be, incorporated as fully as if reproduced herein in full, for all purposes. Features of various embodiments may be combined with other embodiments within the contemplation of this disclosure. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the disclosure.

1. An intravascular lithotripsy (IVL) catheter assembly, comprising:
a hypotube;
a polymer tube connected to a distal portion of the hypotube; and
a balloon bonded to a distal portion of the polymer tube,
wherein the hypotube, the polymer tube, and the balloon are configured in size and shape to allow at least a portion of the IVL catheter assembly to be inserted into a coronary vessel, and
wherein a bending stiffness of the polymer tube is less than or equal to 20 g/mm.

2. The IVL catheter assembly according to claim 1, wherein the bending stiffness is less than or equal to 16.7 g/mm.

3. The IVL catheter assembly according to claim 1, wherein the bending stiffness is measured at a position within a range from the balloon to 12 inches from a distal tip of the IVL catheter assembly.

4. The IVL catheter assembly according to claim 1, wherein the bending stiffness is an average of forces, which displace the polymer tube by 5 mm.

5. The IVL catheter assembly according to claim 1, wherein the polymer tube forms a port, which is to receive a guidewire, in a middle portion thereof.

6. The IVL catheter assembly according to claim 5, wherein a bending stiffness measured at the port is higher than a bending stiffness measured at 12 inches from a distal tip of the IVL catheter assembly.

7. The IVL catheter assembly according to claim 5, wherein a wall of the polymer tube comprises two or more lumens distal to the port.

8. The IVL catheter assembly according to claim 7, wherein the two or more lumens includes lumens for two wire conductors, saline solution, and the guidewire to pass up to a portion, to which the balloon is bonded.

9. The IVL catheter assembly according to claim 1, wherein an outer wall of the polymer tube thickens from a distal tip of the hypotube.

10. The IVL catheter assembly according to claim 1, wherein the polymer tube distal of the hypotube has no support structure other than a material of the polymer tube.

11. An intravascular lithotripsy (IVL) catheter assembly comprising:
a hypotube;
a polymer tube connected to a distal portion of the hypotube; and
a balloon bonded to a distal portion of the polymer tube,
wherein the hypotube, the polymer tube, and the balloon are configured in size and shape to allow at least a portion of the IVL catheter assembly to be inserted into a coronary vessel,
wherein the polymer tube forms a port, which is to receive a guidewire, in a middle portion thereof, and
wherein, distal to the port and proximal to the balloon, the polymer tube forms two or more lumens.

12. The IVL catheter assembly according to claim 11, wherein an outer wall of the polymer tube thickens from a distal tip of the hypotube to the port.

13. The IVL catheter assembly according to claim 11, wherein an outer wall of the polymer tube enlarges in a dimension transverse to a longitudinal axis and includes the two or more lumens.

14. The IVL catheter assembly according to claim 11, wherein the two or more lumens are selected from a first lumen for a first wire conductor, a second lumen for a second wire conductor, a third lumen for saline solution, and a fourth lumen for the guidewire.

15. The IVL catheter assembly according to claim 11, wherein, proximal to the port, the polymer tube forms an empty space therein.

16. The IVL catheter assembly according to claim 11, wherein, a size of one lumen of the two or lumens is increased from the port toward a distal portion of the polymer tube.

17. The IVL catheter assembly according to claim 16, wherein the one lumen is for saline solution to pass therethrough.

18.-19. (canceled)

20. An intravascular lithotripsy (IVL) catheter assembly comprising:
a hypotube;
a polymer tube connected to a distal portion of the hypotube; and
a balloon bonded to a distal portion of the polymer tube,
wherein the hypotube, the polymer tube, and the balloon are configured in size and shape to allow at least a portion of the IVL catheter assembly to be inserted into a coronary vessel, and
wherein the polymer tube forms at least two lumens for two wire conductors, and
wherein the two lumens are incorporated within an outer wall of the polymer tube.

21. An intravascular lithotripsy (IVL) catheter assembly, comprising:
a hypotube;
a polymer tube connected to a distal portion of the hypotube; and
a balloon bonded to a distal portion of the polymer tube,
wherein the hypotube, the polymer tube, and the balloon are configured in size and shape to allow at least a portion of the IVL catheter assembly to be inserted into a coronary vessel, and
wherein a bending stiffness of the polymer tube is less than or equal to 20 g/mm without a mandrel disposed between a distal end of the hypotube and a proximal end of the balloon.

22.-30. (canceled)

31. An intravascular lithotripsy (IVL) catheter assembly, comprising:
a hypotube;
a polymer tube connected to a distal portion of the hypotube; and
a balloon bonded to a distal portion of the polymer tube,
wherein the hypotube, the polymer tube, and the balloon are configured in size and shape to allow at least a portion of the IVL catheter assembly to be inserted into a coronary vessel, and
wherein a bending stiffness of the polymer tube is less than or equal to about 15 g/mm at a position, where a majority of the bending stiffness is provided by a first polymer forming a wall of the polymer tube.

32.-40. (canceled)
